(19) United States
(12) Patent Application Publication
(10) Pub. No.: US 2015/0216985 A1
(43) Pub. Date: Aug. 6, 2015

(54) ENDOSPORE COMPOSITIONS AND USES THEREOF

(71) Applicant: Annuary Healthcare Inc., La Jolla, CA (US)

(72) Inventors: John A. Macoviak, La Jolla, CA (US); Wolfgang Wrasidlo, La Jolla, CA (US); John Norton, San Diego, CA (US); Kristin Spivey, San Diego, CA (US); Steve Oldenberg, San Diego, CA (US); Aaron Saunders, San Diego, CA (US)

(21) Appl. No.: 14/615,117

(22) Filed: Feb. 5, 2015

Related U.S. Application Data
(63) Continuation of application No. 14/106,419, filed on Dec. 13, 2013, now abandoned.
(60) Provisional application No. 61/737,680, filed on Dec. 14, 2012.

(51) Int. Cl. A61K 47/32; A01N 59/16; A61K 33/24; A61K 33/38; A61K 47/10; A61K 9/51; A61K 31/341; A01N 43/08; A61K 31/7028; A01N 43/16; A01N 57/36; A61K 31/665; A01N 43/90; A61K 31/34; A61K 31/496; A01N 43/60; A61K 31/4709; A01N 43/42; A01N 25/24

(52) U.S. Cl. CPC A61K 47/32; A01N 25/24; A01N 59/16; A61K 33/24; A61K 33/38; A61K 47/10; A61K 9/51; A61K 31/341; A01N 43/08; A61K 31/7028; A01N 43/16; A01N 57/36; A61K 31/665; A01N 43/90; A61K 31/34; A61K 31/496; A01N 43/60; A61K 31/4709; A01N 43/42

(57) ABSTRACT

Provided herein in some embodiments is a composition that comprises (a) a nanoscale particle(s) or a microscale particle(s); and (b) a film-forming polymer. Also provided herein is a method for an immediate and sustained released formulation suitable for topical administration or administration to surfaces. Further provided herein in certain embodiments is a method of reducing the population of pathogenic microorganisms on skin or surfaces, wherein a method comprises applying to the skin or surface a composition, wherein the composition comprises (a) a nanoscale particle(s) or a microscale particle(s); and (b) a film-forming polymer.

### CROSS-REFERENCE

This application is a continuation of co-pending U.S. application Ser. No. 14/106,419 filed Dec. 13, 2013, which claims the benefit of U.S. Provisional Application No. 61/737,680 filed on Dec. 14, 2012, entitled, “ENDOSPORE COMPOSITIONS AND USES THEREOF,” which is incorporated herein by reference in its entirety.

### BACKGROUND

Microorganisms are responsible for a number of diseases and adverse conditions. It is generally understood that the majority of microbial pathogens (bacteria, fungi, yeast, molds, viruses, and protozoa) that cause disease gain entry into a mammal through various portals (eyes, ears, nose, mouth), and that these microbes are generally introduced into these portals by the hands. In addition, various types of microbial pathogens are acquired by direct contact with contaminated surfaces in the environment.

Materials and methods for preventing microbial contamination and infectious diseases are needed. A large number of illnesses may be prevented or alleviated by the decontamination of skin and surrounding surfaces.

The occurrence of healthcare-associated infections, also termed nosocomial infections, are of increasing concern both because of the danger posed to patients, as well as the significant direct costs imposed on hospitals and the healthcare system. The Centers for Disease Control and Prevention (CDC) estimates the annual cost of dealing with these cases to be between $1-$1.6 billion, with other estimates proposing as much as $3 billion annually in costs related to bacterial infections (e.g., Clostridium difficile infection). Additionally, evidence suggests that both the number of occurrences and the severity of bacterial infection outbreaks are increasing.

Because of the rising prevalence and cost associated with the treatment of healthcare-associated infections, there is clearly a need for anti-microbial products that effectively kill the bacteria associated with these infections (e.g., C. difficile) and reduce the risk of outbreaks in healthcare facilities, ideally without the use of antibiotics or caustic chemicals.

### SUMMARY OF THE INVENTION

In accordance with the subject matter described herein in some embodiments, provided is a composition comprising (a) a germinant; (b) a nanoscale or microscale particle; (c) a film-forming polymer; and (d) a carrier. Also provided herein in certain embodiments is a method of reducing the population of pathogenic microorganisms on skin or surfaces, including for example hard surfaces. In some embodiments, the composition is not absorbed through the skin. Also provided herein in certain embodiments is a composition comprising (a) a germinant; (b) a nanoscale or microscale particle; (c) a film-forming polymer; and (d) a carrier effective in killing vegetative bacteria. Also provided herein in certain embodiments is a composition comprising (a) a germinant; (b) a nanoscale or microscale particle; (c) a film-forming polymer; and (d) a carrier effective in killing partially germinated bacteria. Further provided herein in certain embodiments is a method of reducing the population of pathogenic microorganisms in combination with biocides effective in killing vegetative or partially germinated bacteria.

In some embodiments, provided is a composition wherein the germinant modulates one or more proteins selected from the group that comprises cwlJ, sleB, cwlD, spoVAC, spoVAD, spoVAE, SecA, LsrB, RelA, SpoT, DksA, CspA/CspC, CspA/CspB, PrkC, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof.

In some embodiments, provided is a composition that comprises a germinant of a compound of Formula I:

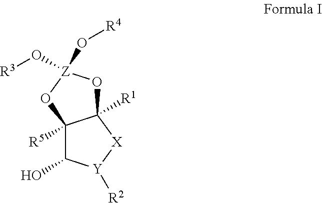

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl; X is O, CH₂, or S;
Y is CH or N; and
Z is C;
or salts thereof;

In some embodiments, provided is a composition that comprises a germinant of a compound of Formula I:

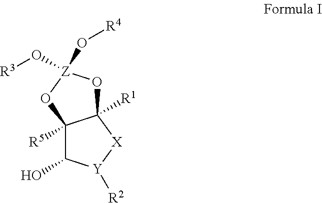

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl; X is O, CH₂, or S;
Y is CH or N; and
Z is B;
or salts thereof;

In some embodiments, provided is a composition that comprises a germinant of a compound of Formula II:

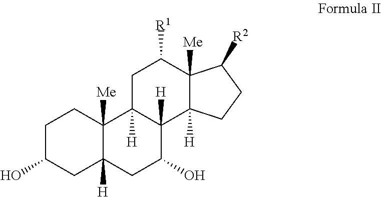

wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
   wherein each R⁶ is independently H or alkyl; or
   has the structure of Formula IIa:

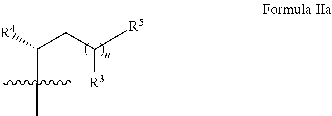

wherein
   each R³ is independently H, alkyl, alkoxy, CO₂R⁶, or halo;
   R⁴ is H, alkyl, OH, alkoxy, or halo;
   R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
   each R⁶ is independently H or alkyl; and
   n is 0, 1, 2, 3, 4, or 5; or
has the structure of Formula IIb:

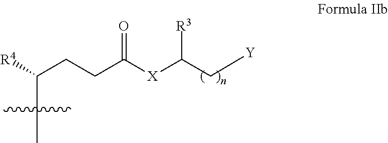

wherein
   R³ is H, alkyl, alkoxy, CO₂R⁶, or halo;
   R⁴ is H, alkyl, OH, alkoxy, or halo;
   X is NR⁶, O or C(R⁶)₂;
   Y is SO₃H, PO₃H, CO₂R⁶, CONR⁶, H, alkyl, OH, alkoxy, halo, aminoalkyl, or amino; and
   each R⁶ is independently H or alkyl;
   n is 0, 1, 2, 3, 4, or 5; or

a salt thereof.

In some embodiments, germinants include, but are not limited to dodecylamine, CaDPA, KCl, Pi, taurocholate, glycine, L-phenylalanine, L-arginine, THMF and ppGppp.

In certain embodiments, the germinant is a selected from the group comprising:
2,3,3,4(2H)-Furantetrol, dihydro-2-methyl-, (2R,4S)-
2,3,3,4(2/-I)-Furantetrol, dihydro-2-methyl-, (2S,4S)-
2,3,3,4(2H)-Furantetrol, dihydro-2-methyl-, (4S)-
2,3,3,4(2H)-Furantetrol, dihydro-2-(methyl-13C)-, (2S,4S)- (9CI)
2,3,3,4(2H)-Furantetrol, dihydro-2-(methyl-13C)-, (2R,4S)- (9CI)
2,3,3,4(2H)-Furantetrol, dihydro-2-(methyl-13C)-, (4S)-
2,3,3,4(2H)-Furantetrol-2-13C, dihydro-2-(methyl-13C)-, (4S)-
2,3,3 (2H)-Furantriol, dihydro-4-methoxy-2-methyl-
2,3,3(2H)-Furantriol, 4-ethoxydihydro-2-methyl
2,3,3(2H)-Furantriol, dihydro-2-methyl-4-propoxy-
3-Furanol, tetrahydro-2,4,4,5-tetramethoxy-5-methyl-
2,3,3,4(2H)-Furantetrol, dihydro-2-(trifluoromethyl)-,(4S)-
2,3,3(2H)-Furantriol, 4-(hexyloxy)dihydro-2-methyl-
2,3,3(2H)-Furantriol, dihydro-2-methyl-4-(phenylmethoxy)-
Borate(1-), [[(2S,3S,4S)-dihydro-2-methyl-2,3,3,4(2H)-furantetrolato(2-)-KO₃,KO₄]dihydroxy-, (T-4)-
3,4-Furandiol, tetrahydro-2,5-dimethoxy-2-methyl- (7CI)
erythro-2-Hexulofuranose, 1,6-dideoxy-5-C-niethoxy-(5E1)-(9CI)
2-Hexulofuranoside, methyl 1,6-dideoxy-5-C-methoxy-, (9CI)
α-D-Psicofuranose, 5-C-hydroxy- (9CI)
β-D-Psicofuranose, 5-C-hydroxy- (9CI)
β-L-Tagatofuranose, 5-C-hydroxy- (9CI)
3,4-Furandiol, tetrahydro-2-(hydroxymethyl)-2,5-dimethoxy- (9CI)
3-Furanol, tetrahydro-2,4,4,5-tetramethoxy-5-methyl-
2-Furoic acid, tetrahydro-3,4-dihydroxy-2,5-dimethoxy-(6CI)
2-Furaldehyde, tetrahydro-3,4-dihydroxy-2,5-dimethoxy-, dimethyl acetal (6CI)
3,4-Furandiol, tetrahydro-2,5-dimethoxy-2-methyl-, diacetate (7CI)
2-Furoic acid, tetrahydro-3,4-dihydroxy-2,5-dimethoxy-, methyl ester (6C1,7CI)
D-erythro-Pentofuranose, 5-deoxy-4-C-hydroxy-2,3-O-(1-methylethylidene)-, (4)-(9CI)
β-D-Fructofuranose, 5-C-hydroxy-, 1-(dihydrogen phosphate) (9CI)
2-Furoic acid, tetrahydro-3,4-dihydroxy-2,5-dimethoxy-5-methyl-, methyl ester (7CI)
2-Furamide, tetrahydro-3,4-dihydroxy-2,5-dimethoxy-(6CI)
Furfuryl alcohol, tetrahydro-3,4-dihydroxy-2,5-dimethoxy-, acetate (6CI)
α-D-Glucopyranoside, 4-C-hydroxy-α-D-arabinofuranosyl (9CI)
Pentofuranoside, methyl 5-deoxy-3-C-(dimethoxymethyl)-4-C-methoxy-(9CI)
D-erythro-Pentofuranoside, methyl 5-deoxy-4-C-hydroxy-2,3-O-(1-methylethylidene)-, (4)- (9CI)
D-erythro-Pentofuranoside, methyl 5-deoxy-4-C-methoxy-2,3-O-(1-methylethylidene)-, (4)- (9CI)
α-D-Fructofuranose, 5-C-hydroxy-, 1,6-bis(dihydrogen phosphate) (9CI)
β-D-Fructofuranose, 5-C-hydroxy-, 1,6-bis(dihydrogen phosphate) (9CI)
α-D-ribo-Hexopyranosid-3-ulose, (4R)-4-C-hydroxy-3-D-arabinofuranosyl (9CI)
α-D-Galactopyranoside, (5S)-5-C-hydroxy-β-D-threo-2-pentulofuranosyl (9CI)
α-D-Glucopyranoside, (5S)-5-C-hydroxy-β-D-threo-2-pentulofuranosyl (9CI)
2,7,12,13-Tetraoxatricyclo[7.2.1.1^{3,6}]tridecane-4,5,9,10,11-pentol, 3-(hydroxymethyl)-1,6-dimethyl-, (4S,5R,10S,11R)-
β-D-Xylofuranose, 1,5-anhydro-4-C-(α-D-glucopyranosyloxy)- (9CI)
2-Furoic acid, tetrahydro-3,4-dihydroxy-2,5-dimethoxy-, -methyl ester, diacetate (7CI)
β-D-threo-2,5-Hexodiulo-2,6-pyranose, 5-hydrate, 5,2¹:5,3¹-dianhydride with 5-C-hydroxy-α-L-sorbofuranose (9CI)
D-erythro-Pentofuranose, 5-bromo-5-deoxy-4-C-hydroxy-2,3-O-(1-methylethylidene)-, (4)-
D-erythro-Pentofuranose, 5-deoxy-4-C-hydroxy-5-iodo-2,3-O-(1-methylethylidene)-, (4)-
3,4-Furandiol, 2-[(benzoyloxy)methyl]tetrahydro-2,5-dimethoxy-(9CI)
β-D-ribo-Pentofuranoside, methyl 5-bromo-5-deoxy-4-C-hydroxy-2,3,O-(1-methylethylidene)-
α-L-lyxo-Pentofuranoside, methyl 5-bromo-5-deoxy-4-C-hydroxy-2,3-O-(1-methylethylidene)-
α-D-ribo-Pentofuranoside, methyl 5-bromo-5-deoxy-4-C-hydroxy-2,3-O-(1-methylethylidene)-
β-L-lyxo-Pentofuranoside, methyl 5-bromo-5-deoxy-4-C-hydroxy-2,3-O-(1-methylethylidene)-
D-erythro-Pentofuranoside, methyl 5-bromo-5-deoxy-4-C-hydroxy-2,3-O-(1-methylethylidene)-, (4)-
D-erythro-Pentofuranoside, methyl 5-deoxy-4-C-hydroxy-5-iodo-2,3-O-(1-methylethylidene)-, (4)-
Pentofuranoside, methyl 5-deoxy-3-C-(dimethoxymethyl)-4-C-methoxy-2,3-bis-O-(trimethylsilyl)- (9CI)
Furo[3,4-d]-1,3,2-dioxaphosphol-4-ol, tetrahydro-2,6-dimethoxy-4,6-dimethyl-, 2-oxide (9CI)
D-erythro-L-ribo-5-Nonuto-5,2-furanose, 5,9-anhydro-1,6,7,8-tetradeoxy-3-O-[(1,1-dimethylethyl)dimethylsilyl]2-C-hydroxy-6,8-dimethyl-4-O-methyl-, (5S)- (9CI)
Pentofuranoside, methyl 5-deoxy-3-C-(dimethoxymethyl)-4-C-methoxy-, diacetate (9CI)
13,14-Dioxatricyclo[8.2.1.1^{4,7}]tetradecane-5,6,11,12-tetrol, 1,4,7,10-tetramethoxy-
3,4-Furandiol, tetrahydro-2,5-dimethoxy-2-methyl-, dibenzoate (7CI)
β-D-Ribofuranoside, (2-nitrophenyl)methyl 4-C-methoxy-
β-L-erythro-Hexofuranosid-5-ulose, methyl 6-deoxy-4-C-methoxy-2,3-O-(1-methylethylidene)-, (S)- (9CI)
α-D-Lyxofuranose, 4-C-ethoxy-5-O-(phenylmethyl)-, triacetate (9CI)
β-D-Lyxofuranose, 4-C-ethoxy-5-O-(phenylmethyl)-, triacetate (9CI)
β-D-Ribofuranoside, (2-nitrophenyl)methyl 4-C-methoxy-2-O-methyl-
α-D-Glucofuranose, 4-0-methyl-1,2:5,6-bis-O-(1-methylethylidene)-2,3,7-Trioxabicyclo[2.2.1]heptane-5,6-diol, 1,4-dimethyl-, dinitrate, (5-endo, 6-exo)- (9CI)
Galactitol, 2,5-anhydro-1,6-dideoxy-2,5-dimethoxy-3,4-di-C-methyl-, cyclic 3,4-(hydrogen phosphate) (8CI)
a-D-Tagatofuranoside, methyl 1,6-dideoxy-5-C-methoxy-3,4-di-C-methyl-, cyclic hydrogen phosphate (9CI)
β-D-Riboruranoside, methyl 4-C-(1-cyanoethoxy)-5-deoxy-2,3-O-(1-methylethylidene)- (9CI)
Phosphoric acid, methyl ester, cyclic 3,4-ester with tetrahydro-5-methoxy-2,3,4,5-tetramethyl-2,3,4-furantriol (7CI)
α-L-Tagatofuranose, 2,5-anhydro-1,6-dideoxy-5-C-methoxy-3,4-di-C-methyl-, cyclic 3,4-(methyl phosphate), (R)-(9CI)
α-L-Tagatofuranose, 1,6-dideoxy-5-C-methoxy-3,4-di-C-methyl-, cyclic 3,4-(methyl phosphate), (S)- (9CI)
Fructofuranose, O-α-D-galactopyranosyl-(1→6)-O-α-D-glucopyranosyl-6-t-(1→4)-, β-D- (8CI)
Raffinose-6′-t (8CI)
SaH-Oxireno[8,8a]naphtho[2,3-b]furan-5a,6-diol, decahydro-7,8a-dimethoxy-4,4a,6-trimethyl-, (1aR,4S,4aR,5aR,6S,7S,8aS,9aS)-Molybdate(1-), [μ-[1,6-dideoxy-5-C-hydroxy-3,4-di-C-methyl-α-psicofuranosato(3-)-O²,O³:O³,O⁴]]-μ-oxotetraoxodi-, steroisomer (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-) (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-), compd. with 1,1′-oxybis[ethane](2:1) (9CI)
9a H-4a,8-Epoxy-1,3-dioxolo[4,5]furo[2,3-d]oxepin-9a-ol, hexahydro-2,2,8-trimethyl-, [3aS-(3aα,4aα,8α,9aβ,9bα)]-(9CI)
α-Lyxofuranoside, methyl 3-C-[(benzoyloxy)methyl]-5-deoxy-4-C-methoxy-, 2-acetate (9CI)
2-Furoic acid, tetrahydro-3,4-dihydroxy-2,5-dimethoxy-, methyl ester, dibenzoate (7CI)
β-D-ribo-Heptofuranoside, methyl 4,6-anhydro-5,7-dideoxy-4-C-hydroxy-6-Cmethyl-2,3-O-(1-methylethylidene)- (9CI)
β-L-Sorbofuranose, 5-C-hydroxy-1,3:4,6-bis-O-(phenylmethylene)-, [1(R),4(R)]- (9CI)
β-L-Sorbofuranose, 5-C-hydroxy-1,3:4,6-bis-O-(phenylmethylene)-(9CI)
α-D-Glucofuranose, 4-O-methyl-1,2:5,6-bis-O-(1-methylethylidene)-, 3-acetate
α-D-Galactofuranose, 4-O-methyl-1,2:5,6-bis-O-(1-methylethylidene)-, 3-acetate
2,4,6-Metheno-2H-cyclopenta[g]furo[2,3,4-ij][2]benzopyran-2,5a,6a,9a,9b,9c(2aH,6H,7H)-hexol, tetrahydro-2a,6,9-trimethyl-4-(1-methylethyl)-, (2S,2aS,4S,5aR,6S,6aS,9R,9aS,9bR,9cS,10S)- (9CI)
Furo[2,3-d:4,5-d]bis[1,3]dioxole, tetrahydro-2,2,3a,6,6,7b-hexamethyl-, (3aα,4aα,7aβ,7bβ)- (9CI)
1,4a-(Epoxymethano)-4aH-xanthen-9(2H)-one, 1,3,4,9a-tetrahydro-1,9a-dihydroxy-11-methoxy-
α-L-Sorbofuranose, 5-C-methoxy-1,3:4,6-bis-O-(phenylmethylene)-, [1(R),4(R),5S]- (9CI)
Furo[2,3-d:4,5-d]bis[1,3]dioxole, tetrahydro-2,2,3a,4a,6,6-hexamethyl-, (3aα,4aμ,7aβ,7bα)- (9CI)
Spiro[furan-2(3H),4′(3′aH)-furo[3,4-d][1,3]dioxole], 6′,6′a-dihydro-6′-methoxy-2′,2′-dimethyl-, (2R,3′aS,6′R,6′aR)-
Octofuranosiduronic acid, methyl 3,6-anhydro-5-deoxy-4-C-methoxy-6-C-(methoxycarbonyl)-, methyl ester (9CI)
β-L-erythro-Hexofuranosid-5-ulose, methyl 6-deoxy-4-C-methoxy-2,3-O-(1-methylethylidene)-, oxime, (4R)- (9CI)
2-Propanone, 1-hydroxy-1-[tetrahydro-6-hydroxy-2,3a,5-trimethyl-5,2-(epoxymethano)furo[2,3-d]-1,3-dioxol-8-yl]-, [2R-[2α,3aβ,5α,6β,6aβ,8S*(R*)]]- (9CI)
β-L-erythro-β-L-lyxo-Decofuranos-7-ulo-7,10-furanose, 10-C-(acetyloxy)-3,7-anhydro-5,6-dideoxy-, tetraacetate, (10R)- (9CI)
Inulobiose, octaacetate (5CI)
2,3,7-Trioxabicyclo[2.2.1]heptane-1-acetonitrile, 5-[[(1,1-dimethylethyl)dimethylsilyl]oxy]-6-methoxy-4-methyl-, (1R,4S,5R,6R)-
D-gluco-Nonitol, 2,5-anhydro-1,6,7,8-tetradeoxy-3-O-[(1,1-dimethylethyl)dimethylsilyl]-2,5-C-epidioxy-6,8-dimethyl-4-O-methyl-, (2,5)- (9CI)
β-D-Lyxofuranose, 4-C-ethoxy-2,3-O-(1-methylethylidene)-5-O-(phenylmethyl)-, acetate (9CI)
2-Heptenal, 6-[(5S,6R)-5-hydroxy-6-methoxy-4-methyl-2,3,7-trioxabicyclo[2.2.1]hept-1-yl]-4-methyl-, (2E,4S,6R)-
Spiro[1,2-dioxin-3 (6H),5′(3′aH)-furo[2,3-d][1,3]dioxole]-6,6′-diol, 6′,6′a-dihydro-2′,2′,6-trimethyl-, 6′-acetate, (3R,3′aS,6′R,6′aR)-
Spiro[1,2-dioxin-3(6H), 5′(3′aH)-furo[2,3-d][1,3]dioxole]-6,6′-diol,
6′,6′a-dihydro-2′,2′,6-trimethyl-, 6′-acetate, (3S,3′aS,6′R,6′aR)-
5,8-Epoxy-1,4-dioxino[2,3-d][1,2]dioxin, hexahydro-4a,5,8,8a-tetramethyl-, (4aR,5S,8R,8aS)-rel-
5,8-Epoxy-1,4-dioxino[2,3-d][1,2]dioxin, hexahydro-4a,5,8,8a-tetramethyl-, (4aR,5R,8S,8aS)-rel-
5,8-Epoxy-1,4-dioxino[2,3-d][1,2]dioxin, hexahydro-4a,5,8,8a-tetramethyl-
4H-1-Benzopyran-4-one, 2-(3,4-dihydroxyphenyl)-5,6-dihydroxy-7-[(4-C-hydroxy-α-D-ribofuranosyl)oxy]- (9CI)
Furo[2,3-d:4,5-d]bis[1,3]dioxole, tetrahydro-2,2,3a,4a,6,6,7a,7b-octamethyl-, (3aα,4aα,17aβ,7bβ)- (9CI)
6,9-Epoxy-2H-o-dioxino[4,5-b][1,4]dioxepin, hexahydro-5a,6,9,9a-tetramethyl-, stereoisomer (8CI)
6,9-Epoxy-2H-o-dioxino[4,5-b][1,4]dioxepin, hexahydro-5a,6,9,9a-tetramethyl-, stereoisomer (8CI)
L-gulo-Nonose, 5,8-anhydro-2,3,4,9-tetradeoxy-7-O-[(1,1-dimethylethyl)dimethylsilyl]-5,8-C-epidioxy-2,4-dimethyl-6-O-methyl-(5,8)- (9CI)
α-L-Sorbofuranose, 5-C-methoxy-1,3:4,6-bis-O-(phenylmethylene)-, acetate, [1(R),4(R),5S]- (9CI)
Spiro[1,2-dioxin-3(6H),5′ (3′aH)-furo[2,3-d)[1,3]dioxol]-6-ol, 6′,6′a-dihydro-6′-methoxy-6-methyl-2′-(trichloromethyl)-, (2′S,3S,3′aS,6′R,6′aR)-
Spiro[1,2-dioxin-3(6H),5′(3′aH)-furo[2,3-d)[1,3]dioxol]-6-ol, 6′,6′a-dihydro-6′-methoxy-6-methyl-2′-(trichloromethyl)-, (2′R,3R,3′aS,6′R,6′aR)-
7,10-Epoxy[1,2]dioxino[4,5-b][1,4]dioxocin, octahydro-6a,7,10,10a-tetramethyl-
Furo[2,3-d:4,5-d]bis[1,3]dioxole-3a(4aH)-carboxylic acid, dihydro-2,2,6,6-tetramethyl- (9CI)
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, (3aR,3′aS,3bR,5S,6′S,6′aR,7aS,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, (3aR,3′aS,3bR,5S,6′S,6′aR,7aR,8aR)- (9CI)
Spiro[1,2-dioxin-3 (6H),5′(3′aH)-furo[2,3-d][1,3]dioxole]-6,6′-diol, 6′,6′a-dihydro-2′,2′,6-trimethyl-, 6,6′-diacetate, (3S,3′aS,6′R,6′aR)-
Spiro[1,2-dioxin-3 (6H),5′(3′aH)-furo[2,3-d][1,3]dioxole]-6,6′-diol, 6′,6′a-dihydro-2′,2′,6-trimethyl-, 6,6′-diacetate, (3R,3′aS,6′R,6′aR)-
2H-Naphtho[1,2-b]pyran-2-one, 6-[[5-C-(α-D-glucopyranosyloxy)-β-D-fructofuranosyl]oxy]-7-hydroxy-3-methyl-
Spiro[furo[3,4-d]-1,3-dioxole-4(3aH),5′(4′H)-isoxazole], 6,6a-dihydro-6-methoxy-2,2,3′-trimethyl-, (3aS,4R,6R,6aR)-
Spiro[1,2-dioxin-3 (6H),5′(3′aH)-furo[2,3-d][1,3]dioxole]-6,6′-diol, 6′,6′a-dihydro-6-methyl-2′-(trichloromethyl)-, 6′-acetate, (2′S,3S,3′aS,6′R,6′aR)-
Spiro[1,2-dioxin-3(6H), 5′(3′aH)-furo[2,3-d][1,3]dioxole]-6,6′-diol, 6′,6′a-dihydro-6-methyl-2′-(trichloromethyl)-, 6′-acetate, (2′R,3R,3′aS,6′R,6′aR)-
α-D-Glucofuranose, 4-C-hydroxy-1,2:5,6-bis-O-(1-methylethylidene)-,
3-(3-chlorobenzoate)
Spiro[5H-1,3-dioxolo[4,5]furo[3,2-b]pyran-5,5′(3′aH)-furo[2,3-d][1,3]dioxol]-6′-ol, 3a,6,6′,6′a,7,8a-hexahydro-2,2,2′,2′-tetramethyl-, (3aR,3′aS,5S,6′S,6′aR,8aR)-
Spiro[furo[3,4-d]-1,3-dioxole-4(3aH),5′(4′H)-isoxazole], 3′-ethyl-6,6a-dihydro-6-methoxy-2,2-dimethyl-, (3aS,4R,6R,6aR)-
1,4-Epoxy-o-dioxino[4,5-b][1,4]benzodioxin, 1,4,4a, 10a-tetrahydro-1,4,4a, 10a-tetramethyl-, stereoisomer (8CI)
1,4-Epoxy-o-dioxino[4,5-b][1,4]benzodioxin, 1,4,4a, 10a-tetrahydro-1,4,4a, 10a-tetramethyl-, stereoisomer (8CI)
1,4:6,9-Diepoxy-p-dioxino[2,3-d:5,6-d]bis-o-dioxin, octahydro-1,4,4a,5a,6,9,9a,10a-octamethyl-, stereoisomer (8CI)
Spiro[5H-1,3-dioxolo[4,5]furo[3,2-b]pyran-5,5′(3′aH)-furo[2,3-d][1,3]dioxol]-6′-ol,6′-(aminomethyl)octahydro-2,2,2′,2′-tetramethyl-, (3aR,3′aS,3bR,5S,6′S,6′aR,7aS,8aR)- (9CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2,2′-tetramethyl-, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, 6′-acetate, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol,6′-(1-aminoethyl)-3a,3b,6,6′,6′a,7,7a,8a-octahydro-2,2,2′,2′-tetramethyl-, (3aR,3′aS,3bS,5S,6′S,6′aR,7aR,8aR)-
L-glycero-β-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-β-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
β-L-threo-Pentofuranose, 4-C-(acetyloxy)-5-deoxy-5-fluoro-1,2-O-(1-methylethylidene)-, 4-methylbenzenesulfonate, (4ξ)- (9CI)
α-D-Glucopyranoside, methyl 6-deoxy-4-O-(4-C-hydroxy-2,3,5-tri-O-methyl-α-D-arabinofuranosyl)-2,3-di-O-methyl-6-[[(4-nitrophenyl) sulfonyl]amino]-
Spiro[furo[3,4-d]-1,3-dioxole-4(3aH),5′(4′H)-isoxazole], 6,6a-dihydro-6-methoxy-2,2-dimethyl-3′-[(3 aR,4R,6R,6aR)-tetrahydro-6-methoxy-2,2-dimethylfuro[3,4-d]-1,3-dioxol-4-yl]-, (3aS,4R,6R,6aR)
Spiro[furo[3,4-d]-1,3-dioxole-4(3aH),2′(3′H)-pyrrolo[1,2-b]isoxazole], hexahydro-6-methoxy-4′,5′-bis(methoxymethoxy)-2,2-dimethyl-, (2′R,3aS,3′aS,4′S,5′S,6R,6aR)-
5,2,9-Ethanylylidene-1-benzoxepin-8,11(2H)-dione, 4-[(2R,3R)-3,4-dihydro-3,5,7-trihydroxy-2H-1-benzopyran-2-yl]-6-[(2S,3S)-3,4-dihydro-3,5,7-trihydroxy-2H-1-benzopyran-2-yl]-5,5a,9,9a-tetrahydro-2,9,9a,10-tetrahydroxy-, (2R,5S,5aS,9S,9aS,10R)- (9CI)
Spiro[5H-1,3-dioxolo[4,5]furo[3,2-b]pyran-5,5′(3′a H)-furo[2,3-d][1,3]dioxol]-6′-ol, 3a,6,6′,6′a,7,8a-hexahydro-2,2,2′,2′-tetramethyl-, 6′-acetate, (3aR,3′aS,5S,6′S,6′aR,8aR)-
Spiro[furo[3,4-d]-1,3-dioxole-4(3aH),5′(4′H)-isoxazole], 6,6a-dihydro-6-methoxy-2,2-dimethyl-3′-phenyl-, (3aS,4R,6R,6aR)-
1,4-Epoxycyclobuta[5,6]-p-dioxino[2,3-d]-o-dioxin, 1,4,4a,5a,7a,8a-hexahydro-1,4,4a,5a,6,7,7a,8a-octamethyl-, stereoisomer (8CI)
1,4-Epoxycyclobuta[5,6]-p-dioxino[2,3-d]-o-dioxin, 1,4,4a,5a,7a,8a-hexahydro-1,4,4a,5a,6,7,7a,8a-octamethyl-, stereoisomer (8CI)
Spiro[furo[3,4-d]-1,3-dioxole-4(3aH),6′-[6H]1,2]oxazine]-3′-carboxylic acid, hexahydro-6-methoxy-2,2-dimethyl-, ethyl ester, (3′ R,3a5,4R,6R,6aR)-
Spiro[furo[3,4-d)-1,3-dioxole-4(3aH),5′-isoxazolidine], dihydro-6-methoxy-2,2-dimethyl-2′-(phenylmethyl)-, (3aS,4R,6R,6aR)-
2H-Naphtho[1,2-b]pyran-2-one, 7-(acetyloxy)-3-methyl-6-[[1,3,4,6-tetra-O-acetyl-5-C-[(2,3,4,6-tetra-O-acetyl-α-D-glucopyranosyl)oxy]-β-D-fructofuranosyl]oxy]-
Spiro[furo[3,4-d)-1,3-dioxole-4(3aH),5′(4′H)-isoxazole), 6,6a-dihydro-6-methoxy-2,2-dimethyl-3′-(4-methylphenyl)-, (3aS,4R,6R,6aR)-
3,6-Epoxy-2H,8H-pyrimido[6,1-b][1,3]-oxazocine-8,10(9H)-(dione, 3,4,5,6-tetrahydro-4-hydroxy-, acetate (ester)(7CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, 6′-benzoate, (3aR,3′aS,3bS,5S,6′S,6′aR,7aR,8aR)-
Spiro[5H-1,3-dioxolo[4,5][furo[3,2-b]pyran-5,5′(3′aH)-furo[2,3-d) [1,3]dioxol]-6′-ol, octahydro-2,2,2′,2′-tetramethyl-6′-(nitromethyl)-, (3aR,3′aS,3bS,5S,6′S,6′aR,7aR,8aR)-
Spiro[furo[3,4-d)-1,3-dioxole-4(3aH),6′-[6H]1,2]oxazine]-3′-carboxylic acid, 4′,5′,6,6a-tetrahydro-6-methoxy-2,2-dimethyl-, ethyl ester, (3aS,4R,6R,6aR)-
Hexanoic acid, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Acetamide, N-[1-[(2′R,3′R,3aS,3′aS,5′S,6S,6aR,7′aR)-2′,3′-bis(acetyloxy)-2′,3′,3′a,6,6′,6a,7′,7′a-octahydro-6-hydroxy-2,2-dimethylspiro[furo[2,3-d]-1,3-dioxole-5(3aH),5′-[5H]furo[3,2-b]pyran]-6-yl]ethyl]-
Spiro[5H-1,3-dioxolo[4,5]furo[3,2-b]pyran-5,5′(3′aH)furo[2,3-d)[1,3]dioxol]-6′-ol, 3a6,6′,6′a,7,8a-hexahydro-2,2,2′,2′-tetramethyl-6′-(nitromethyl)-, (3aR,3′aS,5S,6′S,6′aR,8aR)-
Spiro[5H-1,3-dioxolo[4,5]furo[3,2-b]pyran-5,5′(3′aH)furo[2,3-d)[1,3]dioxol]-6′-ol, 3a,6,6′,6′a,7,8a-hexahydro-2,2,2′,2′-tetramethyl-, 6′-benzoate, (3aR,3′aS,5S,6′S,6′aR,8aR)-
Spiro[furo[3,4-d)-1,3-dioxole-4(3 aH),5′(4′H)-isoxazole], 3′-(2,6-dichlorophenyl)-6,6a-dihydro-6-methoxy-2,2-dimethyl-, (3aS,4R,6R,6aR)-
1,6,11,14,16,20,23,24-Octaoxahexaspiro[3.0.3.0.0.4.0.3.0.3.1.1]tetracosane
Hexanoic acid, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3)dioxol]-6′-yl ester
Benzoic acid, 4-chloro-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)furo[2,3-d[[1,3]dioxol]-6′-yl ester
Benzoic acid, 4-bromo-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)furo[2,3-d][1,3]dioxol]-6′-yl ester
Spiro[5H-1,3-dioxolo[4,5]furo[3,2-b]pyran-5,5′(3′aH)-furo[2,3-d][1,3]dioxol]-6′-ol, 3a6,6′,6′a,7,8a-hexahydro-2,2,2′,2′-tetramethyl-6′-(1-nitroethyl)-, (3′aS3aR,5S,6′S,6′aR,8aR)-
Sorbofuranose, 2,3:4,6-di-O-isopropylidene-, p-toluenesulfonate, α-L-(7CI)
Benzoic acid, 4-chloro-, (3aR,3′aS,5S,6′S,6′aR,8aR)-3a,6,6′,6′a,7,8a-hexahydro-2,2,2,2′-tetramethylspiro[5H-1,3-dioxolo[4,5]furo[3,2-b]pyran-5,5′(3′aH)-furo[2,3-d][1,3]dioxol]-6′-yl ester
Benzoic acid, 4-bromo-, (3aR,3′aS,5S,6′S,6′aR,8aR)-3a,6,6′,6′a,7,8a-hexahydro-2,2,2,2′-tetramethylspiro[5H-1,3-dioxolo[4,5]furo[3,2-b]pyran-5,5′(3′aH)-furo[2,3-d][1,3]dioxol]-6′-yl ester
Spiro[furo[2,3-d]-1,3-dioxole-5(3aH),6′-[6H]1,2]oxazine]-3′-carboxylic acid, hexahydro-2,2-dimethyl-6-(phenylmethoxy)-, ethyl ester, (3′R,3aS,5R,6R,6aR)-
Spiro[furo[3,4-d]-1,3-dioxole-4(3aH),6′-[6H-1,2]oxazine]-2′,3′(3′H)-dicarboxylic acid, tetrahydro-6-methoxy-2,2-dimethyl-, 2′-(1,1-dimethylethyl) 3′-ethyl ester, (3′R,3aS,4R,6R,6aR)-
Spiro[furo[2,3-d]-1,3-dioxole-5(3 aH),6′-[6H]1,2]oxazine]-3′-carboxylic acid, 4′,5′,6,6a-tetrahydro-2,2-dimethyl-6-(phenylmethoxy)-, ethyl ester, (3aS,5R,6R,6aR)-
β-D-Galactofuranose, 3,4-O-[(acetylamino)phenylmethylene]-4-C-hydroxy-, 1-acetate 2,5,6-tribenzoate (9CI)
β-D-Galactofuranose, 3,4-O-[(2,5-dioxo-1-pyrrolidinyl)phenylmethylene]-4-C-hydroxy-, 1-acetate 2,5,6-tribenzoate (9CI)
Osmium, tetraoxotetrakis(pyridine)[μ-[1-(tetrahydro-2,3,4,5-tetrahydroxy-2-furanyl) ethanonato(4-)]]di-, stereoisomer (9CI)
β-D-Galactofuranose, 3,4-O-[(1,3-dihydro-1,3-dioxo-2H-isoindol-2-yl)phenylmethylene]-4-C-hydroxy-, 1-acetate 2,5,6-tribenzoate (9CI)
Piperazinone, 4-[(dihydro-2,2,6,6-tetramethylfuro[2,3-d;4,5-d]bis[1,3]dioxol-3a(4aH)-yl) carbonyl]-6-ethyl-1-[(4-methylphenyl)methyl]- (9CI)
Quinoline, 5,7-dichloro-1-[(dihydro-2,2,6,6-tetramethylfuro[2,3-d:4,5-d]bis[1,3]dioxol-3a(4aH)-yl) carbonyl]-4-(4-fluorophenoxy)-1,2-dihydro-(9CI)

In some embodiments, the composition comprises a film-forming polymer wherein the film-forming polymer is the polyolprepolymer-2. The polyolprepolymer-2 is present in an amount of about 0.05 to about 20% by weight.

In some embodiments, the composition further comprises one or more antimicrobial metal-containing microscale particles having particle sizes of about 10 um to about 1000 um, ethanol, isopropanol, chlorhexidine, chlorhexidine alcohol, iodine, povidone-iodine or Betadine®. In another embodiment, the composition comprises the antimicrobial metal-containing microscale particle having a particle size of about 100 um to about 200 um.

In a further embodiment, an antimicrobial metal-containing microscale particle comprises silver, titanium, zinc, aluminum, iron, copper, platinum, zirconium, palladium, gold, silicon, tungsten salts thereof, or a combination thereof. In some embodiments, the zinc is zinc oxide. In some embodiments, the copper is copper oxide. In some embodiments, the silicon is silicon dioxide. In some embodiments, the silver is silver oxide. In some embodiments, the tungsten is tungsten oxide. In certain specific embodiments, the titanium is titanium dioxide. In other embodiments, the silver microparticle comprises an ionic silver salt, elemental silver, or combinations thereof. In other certain embodiments, the elemental silver is colloidal silver.

In some embodiments, provided is at least one or more antimicrobial metal-containing microscale particles are present in a concentration of about 0.0001% to about 20% by weight.

In some embodiments, the composition comprises one or more antimicrobial metal-containing nanoscale particles having particle sizes of about 1 nm to about 1000 nm, ethanol, isopropanol, chlorihexdine, chlorhexadine alcohol, iodine, povidine-iodine or Betadine®. In some embodiments, the antimicrobial metal-containing nanoscale particle has a particle size of about 100 nm to about 200 nm.

In some embodiments, an antimicrobial metal-containing nanoscale particle comprises silver, titanium, zinc, aluminum, iron, copper, platinum, zirconium, palladium, gold, silicon, tungsten salts thereof, or a combination thereof. In some embodiments, the zinc is zinc oxide. In some embodiments, the copper is copper oxide. In some embodiments, the silicon is silicon dioxide. In some embodiments, the silver is silver oxide. In some embodiments, the tungsten is tungsten oxide. In some embodiments, the titanium is titanium dioxide. In some other embodiments, the silver nanoparticle comprises an ionic silver salt, elemental silver, or combinations thereof. In some specific embodiments, the elemental silver is colloidal silver. In certain embodiments, at least one or more antimicrobial metal-containing nanoscale particles are present in a concentration of about 0.0001% to about 20% by weight.

In some embodiments, the composition comprises a carrier that is suitable for administration to skin or surfaces. In some embodiments, the carrier is selected from the group that comprises tap water, de-ionized water, distilled water, an aqueous solvent system, an aqueous-based single phase liquid solvent system, a hydro-alcoholic solvent system, glycerin, anhydrous liquid solvent, oil, alcohol, and any combinations thereof. In certain specific embodiments, the carrier is ethyl alcohol.

In some embodiments, the composition provides immediate and sustained release of the germinant. In some embodiments, the composition provides immediate and sustained release of the antimicrobial metal-containing nanoscale particle. In some other embodiments, the composition provides immediate and sustained release of the antimicrobial metal-containing microscale particle.

In further embodiments, the composition comprises a coloring agent that adheres to a surface during use to indicate compliant application of the composition.

Provided herein in some embodiments is a composition that comprises (a) a germinant; (b) a antimicrobial metal-containing nanoscale particle or microscale particle; (c) a film-forming polymer; and (d) a carrier. In some embodiments, the composition comprises a germinant that modulates one or more proteins selected from the group that comprises cwlJ, sleB, cwlD, spoVAC, spoVAD, spoVAE, SecA, LsrB, RelA, SpoT, DksA, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof.

In some embodiments, provided is a composition that comprises a germinant of a compound of Formula I:

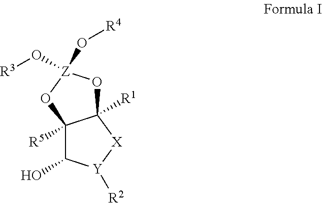

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is C;
or salts thereof.

In some embodiments, provided is a composition that comprises a germinant of a compound of Formula I:

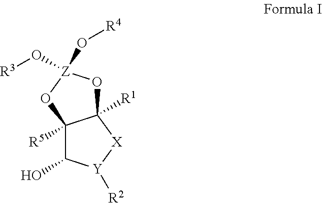

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is B;
or salts thereof.

In some embodiments, provided is a composition that comprises a germinant of a compound of Formula II:

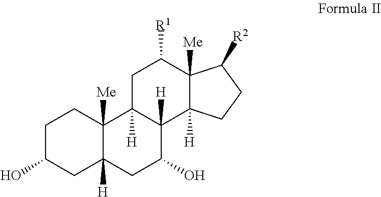

wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
   wherein each R⁶ is independently H or alkyl; or
   has the structure of Formula IIa:

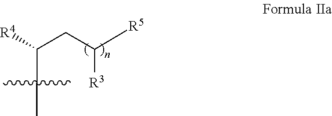

wherein
   each R³ is independently H, alkyl, alkoxy, CO₂R⁶, or halo;
   R⁴ is H, alkyl, OH, alkoxy, or halo;
   R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
   each R⁶ is independently H or alkyl; and
   n is 0, 1, 2, 3, 4, or 5; or
has the structure of formula IIb:

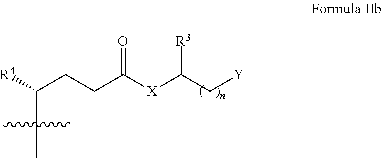

wherein
   R³ is H, alkyl, alkoxy, CO₂R⁶, or halo;
   R⁴ is H, alkyl, OH, alkoxy, or halo;
   X is NR⁶, O or C(R⁶)₂;
   Y is SO₃H, PO₃H, CO₂R⁶, CONR⁶, H, alkyl, OH, alkoxy, halo, aminoalkyl, or amino; and
   each R⁶ is independently H or alkyl;
   n is 0, 1, 2, 3, 4, or 5; or

a salt thereof.

In some embodiments, the composition comprises an antimicrobial metal-containing nanoscale particle having a particle size of about 1 nm to about 1000 nm. In some other embodiments, an antimicrobial metal-containing microscale particle has a particle size of about 10 um to about 1000 um. In other embodiments, an antimicrobial metal-containing nanoscale particle has a particle size of about 100 nm to about 200 nm. In another embodiment, an antimicrobial metal-containing microscale particle has a particle size of about 100 um to about 200 um.

In some embodiments, an antimicrobial metal-containing nanoscale particle comprises silver, titanium, zinc, aluminum, iron, copper, platinum, zirconium, palladium, gold, silicon, tungsten salts thereof, or a combination thereof. In some embodiments, the composition comprising the antimicrobial metal-containing microscale particle comprises silver, titanium, zinc, aluminum, iron, copper, platinum, zirconium, palladium, gold, silicon, tungsten salts thereof, or a combination thereof. In some embodiments, the zinc is zinc oxide. In some embodiments, the copper is copper oxide. In some embodiments, the silicon is silicon dioxide. In some embodiments, the silver is silver oxide. In some embodiments, the tungsten is tungsten oxide. In some embodiments, the titanium is titanium dioxide. In other embodiments, the silver nanoparticle comprises an ionic silver salt, elemental silver, or combinations thereof. In some other embodiments, the silver microparticle comprises an ionic silver salt, elemental silver, or combinations thereof. In some embodiments, the elemental silver is colloidal silver.

In some embodiments, the composition comprises one or more antimicrobial metal-containing nanoscale particles or microscale particles present in a concentration of about 0.0001% to about 20% by weight.

Provided herein in some embodiments, the composition comprises a film-forming polymer that comprises polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or a combination thereof.

In some embodiments, the composition that comprises a film-forming polymer comprises polyolprepolymer-2 (PPG-12/SMDI), poly(styrene-co-maleic anhydride) copolymers (SMA), or a combination thereof. In some embodiments, a film-forming polymer is present in an amount of about 0.05 to about 20% by weight.

Provided herein in some embodiments, the composition comprises a carrier suitable for administration to skin or surfaces. In some embodiments, the carrier is selected from the group that comprises tap water, de-ionized water, distilled water, an aqueous solvent system, an aqueous-based single phase liquid solvent system, a hydro-alcoholic solvent system, glycerin, anhydrous liquid solvent, oil, alcohol, and any combinations thereof. In some further embodiments, the carrier is ethyl alcohol.

Provided herein in some embodiments, the composition provides immediate and sustained release of the germinant. In some embodiments, the composition provides immediate and sustained release of an antimicrobial metal-containing nanoscale particle. In other embodiments, the composition provides immediate and sustained release of an antimicrobial metal-containing microscale particle.

In some further embodiments, the composition comprises a coloring agent that adheres to a surface during use to indicate compliant application of the composition.

Provided herein in some embodiments is a composition that comprises (a) a germinant; (b) a colloidal silver microparticle or nanoparticle; (c) a polyolprepolymer-2; and (d) a carrier. In some embodiments, the composition comprises a that germinant modulates one or more proteins selected from the group that comprises cwlJ, sleB, cwlD, spoVAC, spoVAD, spoVAE, SecA, LsrB, RelA, SpoT, DksA, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof.

In some embodiments, provided is a composition that comprises a germinant of a compound of Formula I:

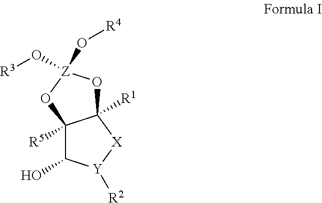

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is C;
or salts thereof.

In some embodiments, provided is a composition that comprises a germinant of a compound of Formula I:

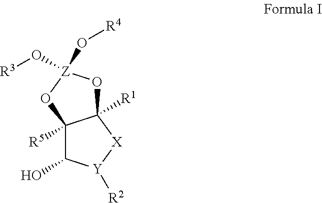

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is B;
or salts thereof.

In some embodiments, provided is a composition that comprises a germinant of a compound of Formula II:

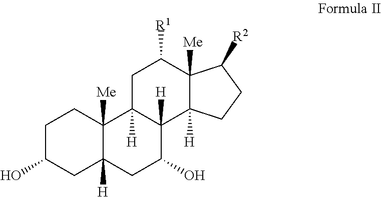

wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
   wherein each R⁶ is independently H or alkyl; or
   has the structure of Formula IIa:

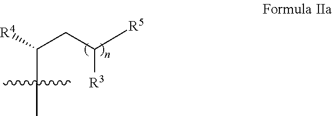

wherein
   each R³ is independently H, alkyl, alkoxy, CO₂R⁶, or halo;
   R⁴ is H, alkyl, OH, alkoxy, or halo;
   R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
   each R⁶ is independently H or alkyl; and
   n is 0, 1, 2, 3, 4, or 5; or
has the structure of Formula IIb:

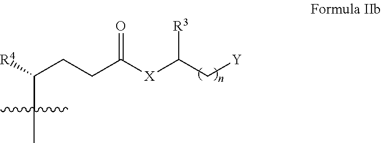

wherein
   R³ is H, alkyl, alkoxy, CO₂R⁶, or halo;
   R⁴ is H, alkyl, OH, alkoxy, or halo;
   X is NR⁶, O or C(R⁶)₂;
   Y is SO₃H, PO₃H, CO₂R⁶, CONR⁶, H, alkyl, OH, alkoxy, halo, aminoalkyl, or amino; and
   each R⁶ is independently H or alkyl;
   n is 0, 1, 2, 3, 4, or 5; or

a salt thereof.

In some embodiments, the composition comprises a colloidal silver nanoscale particle having a particle size of about 1 nm to about 1000 nm. In some embodiments, the composition comprises a colloidal silver microscale particle having a particle size of about 10 um to about 1000 um. In certain embodiments, a colloidal silver nanoscale particle has a particle size of about 100 nm to about 200 nm. In other embodiments, a colloidal silver microscale particle has a particle size of about 100 um to about 200 um. In some embodiments, the composition comprises one or more colloidal silver nanoscale particles or microscale particles present in a concentration of about 0.0001% to about 5% by weight.

In some embodiments, a polyolprepolymer-2 is present in an amount of about 0.05 to about 20% by weight.

In some embodiments, the composition provides a carrier that is suitable for administration to skin or surfaces. In some embodiments, the carrier is selected from the group that comprises tap water, de-ionized water, distilled water, an aqueous solvent system, an aqueous-based single phase liquid solvent system, a hydro-alcoholic solvent system, glycerin, anhydrous liquid solvent, oil, alcohol, and any combinations thereof. In some specific embodiments, the carrier is ethyl alcohol.

In some embodiments, the composition provides immediate and sustained release of the germinant. In some other embodiments, the composition provides immediate and sustained release of the colloidal silver nanoscale particle or microscale particle. In some further embodiments, the composition comprises a coloring agent that adheres to a surface during use to indicate compliant application of the composition.

Provided herein in some embodiments is a bacterial endospore germinating film comprising: (a) a germinants; (b) a polyolprepolymer-2; and (c) a carrier.

In some embodiments, a bacterial endospore germinating film comprises a germinant that modulates one or more proteins is selected from the group that comprises cwlJ, sleB, cwlD, spoVAC, spoVAD, spoVAE, SecA, LsrB, RelA, SpoT, DksA, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof.

In some embodiments, a bacterial endospore germinating film comprises a germinant of a compound of Formula I:

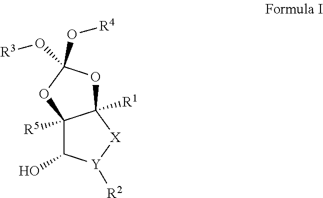

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is C;
or salts thereof.

In some embodiments, a bacterial endospore germinating film comprises a germinant of a compound of Formula I:

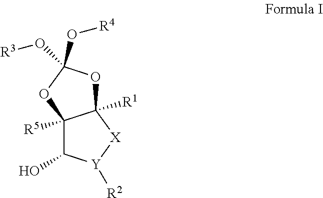

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is B;
or salts thereof.

In some embodiments, a bacterial endospore germinating film comprises a germinant of a compound of Formula II:

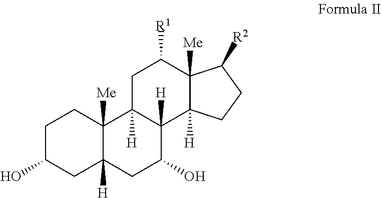

wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
   wherein each R⁶ is independently H or alkyl; or
   has the structure of Formula IIa:

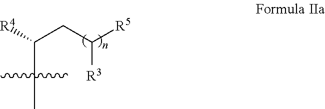

wherein
   each R³ is independently H, alkyl, alkoxy, CO₂R⁶, or halo;
   R⁴ is H, alkyl, OH, alkoxy, or halo;
   R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
   each R⁶ is independently H or alkyl; and
   n is 0, 1, 2, 3, 4, or 5; or
has the structure of Formula IIb:

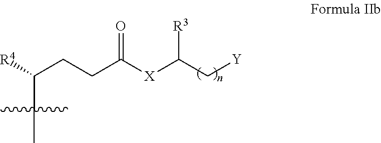

wherein
   R³ is H, alkyl, alkoxy, CO₂R⁶, or halo;
   R⁴ is H, alkyl, OH, alkoxy, or halo;
   X is NR⁶, O or C(R⁶)₂;
   Y is SO₃H, PO₃H, CO₂R⁶, CONR⁶, H, alkyl, OH, alkoxy, halo, aminoalkyl, or amino; and
   each R⁶ is independently H or alkyl;
   n is 0, 1, 2, 3, 4, or 5; or

a salt thereof.

In some embodiments, the bacterial endospore germinating film comprises polyolprepolymer-2 present in an amount of about 0.05 to about 20% by weight. In some embodiments, the bacterial endospore germinating film further comprises one or more antimicrobial metal-containing nanoscale particles having particle sizes of about 1 nm to about 1000 nm. In other embodiments, the bacterial endospore germinating film further comprises one or more antimicrobial metal-containing microscale particles having particle sizes of about 10 um to about 1000 um. In some embodiments, the bacterial endospore germinating film comprises an antimicrobial metal-containing nanoscale particle having a particle size of about 100 nm to about 200 nm. In other embodiments, the bacterial endospore germinating film comprises an antimicrobial metal-containing microscale particle having a particle size of about 100 um to about 200 um.

In some embodiments, the bacterial endospore germinating film comprises an antimicrobial metal-containing nanoscale particle or microscale particle that comprises silver, titanium, zinc, aluminum, iron, copper, platinum, zirconium, palladium, gold, silicon, tungsten salts thereof, or a combination thereof. In some embodiments, the zinc is zinc oxide. In some embodiments, the copper is copper oxide. In some embodiments, the silicon is silicon dioxide. In some embodiments, the silver is silver oxide. In some embodiments, the tungsten is tungsten oxide. In some embodiments, the titanium is titanium dioxide.

In some further embodiments, the bacterial endospore germinating film comprises a silver nanoparticle or microparticle that comprises an ionic silver salt, elemental silver, or combinations thereof. In some embodiments, the elemental silver is colloidal silver.

In some specific embodiments, the bacterial endospore germinating film comprises at least one or more antimicrobial metal-containing nanoscale particles or microscale particles present in a concentration of about 0.0001% to about 20% by weight.

In some embodiments, the bacterial endospore germinating film comprises a carrier suitable for administration to skin or surfaces. In some embodiments, the carrier is selected from the group that comprises tap water, de-ionized water, distilled water, an aqueous solvent system, an aqueous-based single phase liquid solvent system, a hydro-alcoholic solvent system, glycerin, anhydrous liquid solvent, oil, alcohol, and any combinations thereof. In certain specific embodiments, the carrier is ethyl alcohol.

In further embodiments, the bacterial endospore germinating film provides immediate and sustained release of a germinant. In some embodiments, the bacterial endospore germinating film provides immediate and sustained release of the antimicrobial metal-containing nanoscale particle or microscale particle. In some embodiments, the bacterial endospore germinating film comprises a coloring agent that adheres to a surface during use to indicate compliant application of the composition.

Provided herein in some embodiments is a polymeric antimicrobial film that comprises (a) one or more germinants; (b) a antimicrobial metal-containing nanoscale particle; (c) a film-forming polymer; and (d) a carrier. Also provided herein in some embodiments is a polymeric antimicrobial film that comprises (a) a germinant; (b) a antimicrobial metal-containing microscale particle; (c) a film-forming polymer; and (d) a carrier.

In some embodiments, the polymeric antimicrobial film comprises a germinant that modulates one or more proteins selected from the group that comprises cwlJ, sleB, cwlD, spoVAC, spoVAD, spoVAE, SecA, LsrB, RelA, SpoT, DksA, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof.

In some embodiments, a polymeric antimicrobial film comprises a germinant of a compound of Formula I:

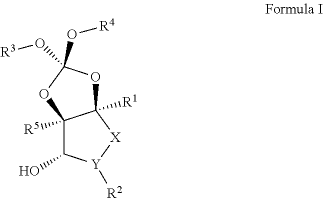

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is C;
or salts thereof.

In some embodiments, a polymeric antimicrobial film comprises a germinant of a compound of Formula I:

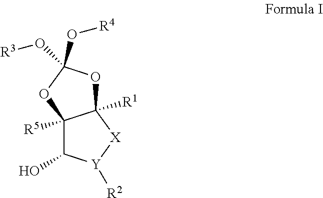

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is B;
or salts thereof.

In other embodiments, a polymeric antimicrobial film comprises a germinant of a compound of Formula II:

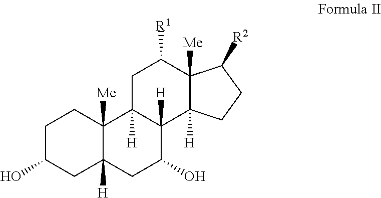

wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
   wherein each R⁶ is independently H or alkyl; or
   has the structure of Formula IIa:

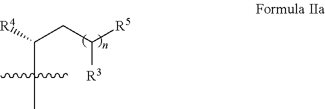

wherein
   each R³ is independently H, alkyl, alkoxy, CO₂R⁶, or halo;
   R⁴ is H, alkyl, OH, alkoxy, or halo;
   R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
   each R⁶ is independently H or alkyl; and
   n is 0, 1, 2, 3, 4, or 5; or
has the structure of Formula IIb:

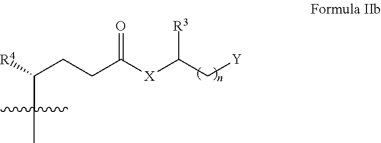

wherein
   R³ is H, alkyl, alkoxy, CO₂R⁶, or halo;
   R⁴ is H, alkyl, OH, alkoxy, or halo;
   X is NR⁶, O or C(R⁶)₂;
   Y is SO₃H, PO₃H, CO₂R⁶, CONR⁶, H, alkyl, OH, alkoxy, halo, aminoalkyl, or amino; and
   each R⁶ is independently H or alkyl;
   n is 0, 1, 2, 3, 4, or 5; or

a salt thereof.

In some embodiments, a polymeric antimicrobial film comprises an antimicrobial metal-containing nanoscale particle having a particle size of about 1 nm to about 1000 nm. In some embodiments, the polymeric antimicrobial film comprises the antimicrobial metal-containing microscale particle having a particle size of about 10 um to about 1000 um.

In some embodiments, the polymeric antimicrobial film comprises an antimicrobial metal-containing nanoscale particle having a particle size of about 100 nm to about 200 nm. In some embodiments, a polymeric antimicrobial film comprises an antimicrobial metal-containing microscale particle having a particle size of about 100 um to about 200 um.

In some embodiments, a polymeric antimicrobial film comprises an antimicrobial metal-containing nanoscale particle or microscale particle comprises silver, titanium, zinc, aluminum, iron, copper, platinum, zirconium, palladium, gold, silicon, tungsten salts thereof, or a combination thereof. In some embodiments, the zinc is zinc oxide. In some embodiments, the copper is copper oxide. In some embodiments, the silicon is silicon dioxide. In some embodiments, the silver is silver oxide. In some embodiments, the tungsten is tungsten oxide. In some embodiments, the titanium is titanium dioxide. In further embodiments, a polymeric antimicrobial film comprises a silver nanoparticle or microparticle that comprises an ionic silver salt, elemental silver or combinations thereof. In some embodiments, the elemental silver is colloidal silver.

In some further embodiments, a polymeric antimicrobial film comprises one or more antimicrobial metal-containing nanoscale particle(s) or microscale particle(s) present in a concentration of about 0.0001% to about 20% by weight.

In some embodiments, a polymeric antimicrobial film comprises a film-forming polymer that comprises polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or a combination thereof.

In some certain embodiments, a polymeric antimicrobial film that comprises a film-forming polymer is polyolprepolymer-2 (PPG-12/SMDI), poly(styrene-co-maleic anhydride) copolymers (SMA), or a combination thereof. In certain specific embodiments, a film-forming polymer is present in an amount of about 0.05 to about 20% by weight.

In some further embodiments, a polymeric antimicrobial film comprises a carrier suitable for administration to skin or surfaces. In some embodiments, the carrier is selected from the group that comprises tap water, de-ionized water, distilled water, an aqueous solvent system, an aqueous-based single phase liquid solvent system, a hydro-alcoholic solvent system, glycerin, anhydrous liquid solvent, oil, alcohol, and any combinations thereof. In certain specific embodiments, the carrier is ethyl alcohol.

In some embodiments, a polymeric antimicrobial film comprises the composition that provides immediate and sustained release of the germinant. In further embodiments, the polymeric antimicrobial film comprises the composition that provides immediate and sustained release of the antimicrobial metal-containing nanoscale particle or microscale particle.

In further embodiments, the polymeric antimicrobial film comprises a coloring agent that adheres to a surface during use to indicate compliant application of the composition.

Further provided herein in some embodiments is a polymeric antimicrobial film that comprises (a) a germinant; (b) a colloidal silver nanoscale particles; (c) a polyolprepolymer-2; and (d) a carrier. Also provided herein in some embodiments is a polymeric antimicrobial film that comprises (a) a germinant; (b) a colloidal silver microscale particles; (c) a polyolprepolymer-2; and (d) a carrier.

In some embodiments, a polymeric antimicrobial film comprises a germinant that modulates one or more proteins selected from the group that comprises cwlJ, sleB, cwlD, spoVAC, spoVAD, spoVAE, SecA, LsrB, RelA, SpoT, DksA, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof.

In some embodiments, a polymeric antimicrobial film comprises a germinant of a compound of Formula I:

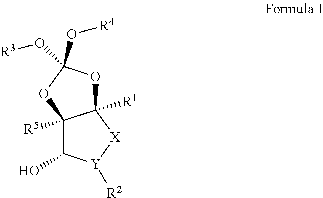

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is C;
or salts thereof.

In some embodiments, a polymeric antimicrobial film comprises a germinant of a compound of Formula I:

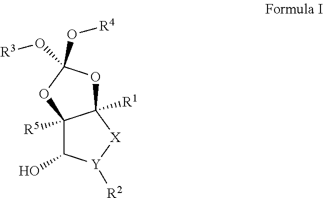

wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl;
X is O, CH₂, or S;
Y is CH or N; and
Z is B;
or salts thereof.

In some embodiments, a polymeric antimicrobial film comprises a germinant of a compound of Formula II:

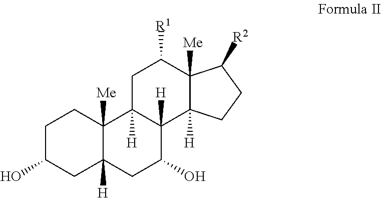

wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
   wherein each R⁶ is independently H or alkyl; or
   has the structure of Formula IIa:

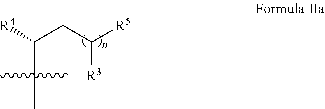

wherein
   each R³ is independently H, alkyl, alkoxy, CO₂R⁶, or halo;
   R⁴ is H, alkyl, OH, alkoxy, or halo;
   R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
   each R⁶ is independently H or alkyl; and
   n is 0, 1, 2, 3, 4, or 5; or
has the structure of Formula IIb:

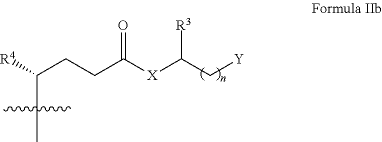

wherein
   R³ is H, alkyl, alkoxy, CO₂R⁶, or halo;
   R⁴ is H, alkyl, OH, alkoxy, or halo;
   X is NR⁶, O or C(R⁶)₂;
   Y is SO₃H, PO₃H, CO₂R⁶, CONR⁶, H, alkyl, OH, alkoxy, halo, aminoalkyl, or amino; and
   each R⁶ is independently H or alkyl;
   n is 0, 1, 2, 3, 4, or 5; or

a salt thereof.

In some embodiments, a polymeric antimicrobial film comprises the colloidal silver wherein the colloidal silver nanoscale particle has a particle size of about 1 nm to about 1000 nm. In other embodiments, a polymeric antimicrobial film comprises the colloidal silver wherein the colloidal silver microscale particle has a particle size of about 10 um to about 1000 um.

In some embodiments, a colloidal silver nanoscale particle has a particle size of about 100 nm to about 200 nm. In other embodiments, the colloidal silver microscale particle has a particle size of about 100 um to about 200 um.

In some further embodiments, a polymeric antimicrobial film comprises the colloidal silver wherein one or more colloidal silver nanoscale particles or microscale particles are present in a concentration of about 0.0001% to about 20% by weight.

In further embodiments, a polymeric antimicrobial film comprises polyolprepolymer-2 wherein polyolprepolymer-2 is present in an amount of about 0.05% to about 20% by weight.

In some embodiments, a polymeric antimicrobial film comprises a carrier wherein the carrier is suitable for administration to skin or surfaces. In further embodiments, the carrier is selected from the group that comprises tap water, de-ionized water, distilled water, an aqueous solvent system, an aqueous-based single phase liquid solvent system, a hydro-alcoholic solvent system, glycerin, anhydrous liquid solvent, oil, alcohol, and any combinations thereof. In some embodiments, the carrier is ethyl alcohol.

In some embodiments, a polymeric antimicrobial film comprises a composition that provides immediate and sustained release of the germinant. In other embodiments, a polymeric antimicrobial film comprises a composition that provides immediate and sustained release of the colloidal silver nanoscale particle or microscale particle.

In further embodiments, a polymeric antimicrobial film comprises a coloring agent that adheres to a surface during use to indicate compliant application of the composition.

Provided herein in certain embodiments is a method of reducing the population of pathogenic microorganisms on a surface, comprising applying to the surface a composition of any one of the aforementioned. In some embodiments, the composition is not absorbed through the skin. Also provided herein in some embodiments is a method of killing at least one pathogenic microorganism on a surface, comprising applying to the surface a composition. Further provided herein in certain embodiments is a method of killing at least one pathogenic microorganism in combination with biocides effective in killing vegetative or partially germinated bacteria. Further provided herein in some embodiments is a method of sterilizing a surface wherein the method comprises applying to the surface a composition.

In some embodiments, the method provides for when the pathogenic microorganisms comprise bacteria, viruses, fungi, or combinations thereof. In some embodiments, the pathogenic microorganism is selected from the group that comprises Aeromonas hydrophila, Aeromonas sobria, Aeromonas caviae, Actinomyces israelii, Actinomyces naeslundii, Bacillus anthracis, Bacillus cereus, Bacillus subtilis, Bacillus thuringiensis, Bacillus stearothermophilus, Bacteroides fragilis, Bordetella pertussis, Bordetella parapertussis, Bordetella bronchiseptica, Borrelia recurrentis, Borrelia burgdorferi, Brucella abortus, Brucella canis, Brucella melintensis, Brucella suis, Burkholderia pseudomallei, Burkholderia cepacia, Campylobacter jejuni, Campylobacter coli, Campylobacter lari, Campylobacter fetus, Clostridium perfringens, Clostridium difficile, Clostridium botulinum, Corynebacterium diphtheria, Corynebacterium jeikeum, Corynebacterium urealyticum, Edwardsiella tarda, Citrobacter freundii Citrobacter diversus, Enterobacter aerogenes, Enterobacter agglomerans, Enterobacter cloacae, Escherichia coli, Klebsiella pneumoniae, Klebsiella oxytoca, Morganella morganii, Proteus mirabilis, Proteus vulgaris, Providencia alcalifaciens, Providencia rettgeri, Providencia stuartii, Salmonella enteric, Salmonella typhi, Salmonella paratyphi, Salmonella enteritidis, Salmonella cholerasuis, Salmonella typhimurium, Serratia marcesans, Serratia liquifaciens, Shigella dysenteriae, Shigella flexneri, Shigella boydii, Shigella sonnei, Yersinia enterocolitica, Yersinia pestis, Yersinia pseudotuberculosis, Enterococcus faecalis, Enterococcus faecium, Erysipelothrix rhusopathiae, Francisella tularensis, Haemophilus influenzae, Haemophilus ducreyi, Haemophilus aegyptius, Haemophilus parainfluenzae, Haemophilus haemolyticus, Haemophilus parahaemolyticus, Helicobacter pylori, Helicobacter cinaedi, Helicobacter fennelliae, Klebsiella pneumoniae, Legionella pneumophila, Listeria monocytogenes, Mycobacterium leprae, Mycobacterium tuberculosis, Nocardia asteroids, Nocardia brasiliensis, Neisseria gonorrhoeae, Neisseria meningitides, Pasteurella multocida, Proteus vulgaris, Proteus mirabilis, Salmonella enteric, Salmonella typhi, Salmonella paratyphi, Salmonella enteritidis, Salmonella cholerasuis, Salmonella typhimurium, Shigella dysenteriae, Shigella flexneri, Shigella boydii, Shigella sonnei, Staphylococcus aureus, Staphylococcus epidermidis, Staphylococcus saprophyticus, Streptococcus pyogenes, Streptococcus agalactiae, Streptococcus anginosus, Streptococcus equismilis, Streptococcus bovis, Streptococcus anginosus, Streptococcus mutans, Streptococcus salivarius, Streptococcus sanguis, Streptococcus mitis, Streptococcus milleri, Treponema pallidum, Treponema pallidum, Treponema pallidum, Treponema carateum, Vibrio cholerae, Vibrio parahaemolyticus, Vibrio vulnificus, Vibrio alginolyticus, Vibrio mimicus, Vibrio hollisae, Vibrio fluvialis, Vibrio metchnikovii, Vibrio damsel, Vibrio furnisii, Yersinia enterocolitica, Yersinia pestis, Yersinia pseudotuberculosis, Rhinovirus, Influenzavirus, Herpes simplex virus, HIV, Ebolavirus, Saccharomyces cerevisiae, Pityrosporum ovale, Malassezia furfur, Candida albicans, Cryptococcus neoformans, Aspergillus, Rhizopus, Mucor or any combination thereof.

In some embodiments, a method provides for when the pathogenic microorganisms comprise endospore-forming bacteria. In some embodiments provides a method of accelerating germination of one or more bacterial endospores on a surface that comprises application to the surface a composition.

In further embodiments, a method provides for bacterial endospores wherein the bacterial endospores comprise Bacillus or Clostridium species, or combinations thereof.

In some certain embodiments, the method provides for when the endospore-forming bacteria comprise Clostridium difficile, Clostridium perfringens, Clostridium sporogenes, Bacillus subtilis, Bacillus circulans, Bacillus pumilus, Bacillus cereus, Bacillus stearothermophilu, Bacillus anthracis, Bacillus globigii, or combinations thereof.

In further embodiments, the method provides for when the surface is substantially free of microorganisms for at least about 6 hours after application of the composition. In yet further embodiments, the method provides for when the surface is non-porous or porous. In specific embodiments, the surface is skin, a medical device, medical equipment, or a medical instrument.

In further embodiments, the method comprises a heating step prior to or after application of composition. In certain specific embodiments, the method comprises a non-heating step prior to or after application of composition.

### SUMMARY OF FIGURES

FIG. 1 is illustrative of the mode of action of certain embodiments of the subject matter described herein.

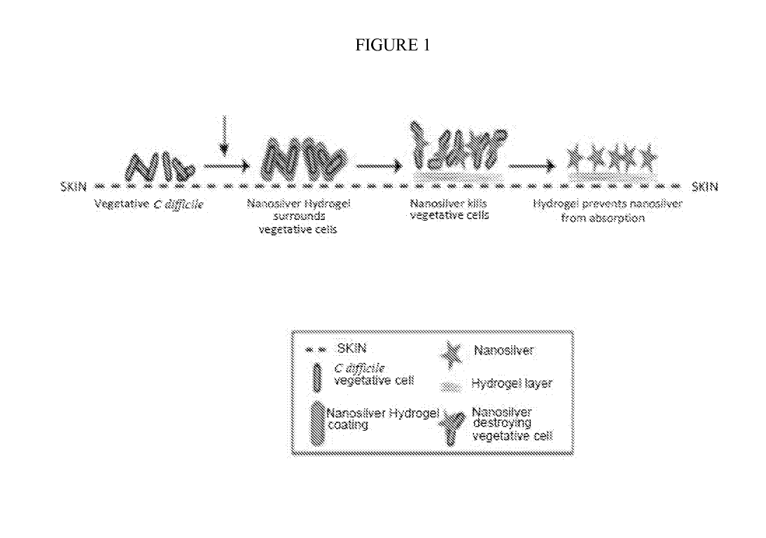

FIG. 2 is illustrative of a comparison between use of certain embodiments of the subject matter compared to control.

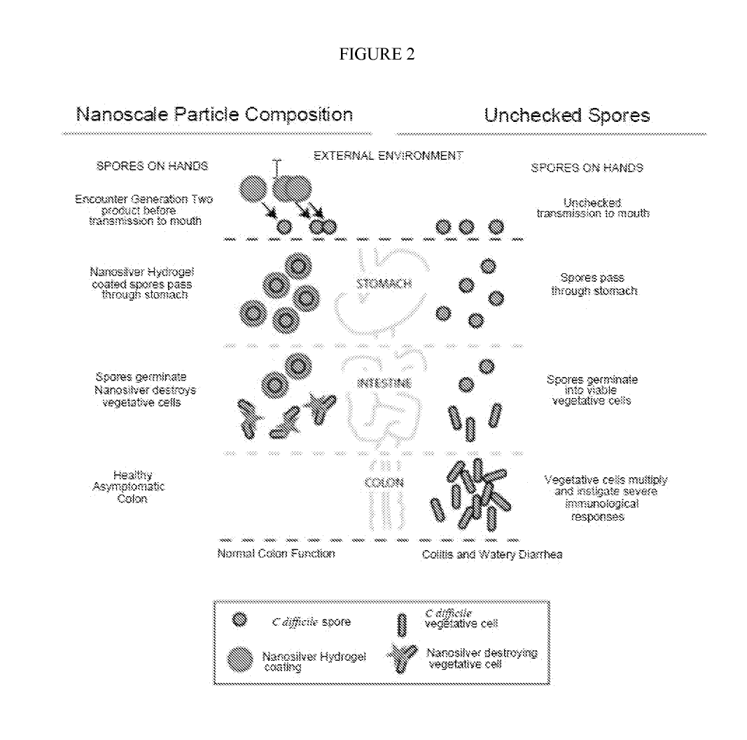

### DETAILED DESCRIPTION OF THE INVENTION

While preferred embodiments have been shown and described herein, it will be apparent to those skilled in the art that such embodiments are provided by way of example only. Numerous variations, changes, and substitutions will now occur to those skilled in the art without departing from the spirit of the disclosure. It should be understood that various alternatives to the embodiments described herein may be employed in practicing the subject matter described herein. It is intended that the following claims define the scope of the disclosure and that methods and structures within the scope of these claims and their equivalents be covered thereby.

Provided herein in some embodiments is a formulation comprising (a) a nanoscale particle; and (b) a film-forming polymer. Provided herein in certain embodiments is a formulation comprising (a) a microscale particle; and (b) a film-forming polymer. In some instances, the formulation is an immediate and sustained released formulation suitable for topical administration or administration to surfaces. Also provided here in certain embodiments is a method of reducing the population of pathogenic microorganisms on skin or surfaces, the method comprising applying to the skin or surface a composition, the composition comprising (a) a nanoscale particle; and (b) a film-forming polymer. Further provided herein, in certain embodiments is a method of reducing the population of pathogenic microorganisms on skin or surfaces, the method comprising applying to the skin or surface a composition, the composition comprising (a) a microscale particle; and (b) a film-forming polymer. Certain embodiments provide a method of killing at least one pathogenic microorganism on the skin or surface, the method comprising applying to the skin or surface a composition, the composition comprising (a) a nanoscale particle; and (b) a film-forming polymer. Certain embodiments provide a method of killing at least one pathogenic microorganism on the skin or surface, the method comprising applying to the skin or surface a composition, the composition comprising (a) a microscale particle; and (b) a film-forming polymer. Also provided herein in certain embodiments is a composition comprising (a) a germinant; (b) a nanoscale or microscale particle; (c) a film-forming polymer; and (d) a carrier effective in killing vegetative bacteria. Also provided herein in certain embodiments is a composition comprising (a) a germinant; (b) a nanoscale or microscale particle; (c) a film-forming polymer; and (d) a carrier effective in killing partially germinated bacteria. Further provided herein in certain embodiments is a method of reducing the population of pathogenic microorganisms in combination with biocides effective in killing vegetative or partially germinated bacteria. In some embodiments, the formulations and compositions described herein are not absorbed through the skin, odor control agents, antimicrobial surface coatings, self-cleaning surface coatings, germicide, antibacterial agents, anti-microbials, anti-fungals, anti-viral agents, anti-protozoal agents, microbiostats, or disinfectants.

### CERTAIN DEFINITIONS

The term “nanoscale particle” includes nanospheres, nanorods, nanofibers, and nanowires. In some embodiments, these nanoscale particles are part of a nano-network.

The term “microscale particle” includes microspheres, microrods, microfibers, and microwires. In some embodiments, these microscale particles are part of a micro-network.

The term “average” dimension of a plurality of nanoparticles means the average of that dimension for the plurality.

The term “photocatalysis” means catalysis that is dependent on the presence of electromagnetic radiation to catalyze a reaction.

The term “visible light” means electromagnetic radiation having a wavelength from 380 nm to 780 nm.

The term “toxinotype” with reference to Clostridium difficile, is based on the variability of the 19.6-kb genomic pathogenicity locus (PaLoc) region coding for TcdA and TcdB (usually detected by restriction endonuclease analysis), wherein there are about 31 toxinotypes.

The term “ribotype” refers to the fingerprint of genomic DNA restriction fragments that contain all or part of the genes coding for the 16S and 23S rRNA, wherein the term is used for identifying and classifying bacteria.

The term “serogroup” refers to antigens expressed on the surface of the bacteria.

In some embodiments, the combination compositions disclosed herein act additively or synergistically. In some embodiments, a “synergistic effect” is seen where the combination of the nanoscale particle and additional active agent(s) results in an activity that is more than the effect of the two individual agents alone.

As used in the specification and the appended claims, the singular forms “a”, “an” and “the” include plural references unless the context clearly dictates otherwise. Thus for example, reference to “the method” includes one or more methods, and/or steps of the type described herein and/or which will become apparent to those persons skilled in the art upon reading this disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “alkyl” as used herein refers to saturated or unsaturated, straight- or branched-chain hydrocarbon radicals derived from a hydrocarbon moiety containing between one and twenty carbon atoms by removal of a single hydrogen atom. Alkyl groups as used herein optionally include one or more further substituent groups. This term is exemplified by groups such as methyl, ethyl, n-propyl, isopropyl, n-butyl, iso-butyl, tert-butyl, n-hexyl, n-octyl, tert-octyl and the like, and are substituted or unsubstituted. The term “lower alkyl” refers to alkyl groups having 1 to 6 carbon atoms. The term “alkyl” also includes “cycloalkyls” as defined below.

The term “cycloalkyl” refers to a monocyclic or polycyclic aliphatic, non-aromatic radical, wherein each of the atoms forming the ring (i.e. skeletal atoms) is a carbon atom. Cycloalkyls may be saturated, or partially unsaturated. Cycloalkyls may be fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, and cyclooctyl. Cycloalkyl groups may be substituted or unsubstituted. Depending on the structure, a cycloalkyl group can be a monoradical or a diradical (i.e., an cycloalkylene group, such as, but not limited to, cyclopropan-1,1-diyl, cyclobutan-1,1-diyl, cyclopentan-1,1-diyl, cyclohexan-1,1-diyl, cyclohexan-1,4-diyl, cycloheptan-1,1-diyl, and the like). In one aspect, a cycloalkyl is a C₃-C₆cycloalkyl.

An “alkoxy” group refers to a (alkyl)O— group, where alkyl is as defined herein.

The term “halo” or, alternatively, “halogen” or “halide” means fluoro, chloro, bromo or iodo.

“Subject” includes humans. The terms “human,” “patient” and “subject” are used interchangeably herein.

“Effective amount” means the amount of a compound that, when administered to a subject for treating a disease, cosmetic or dermatological condition, is sufficient to effect such treatment for the disease, cosmetic or dermatological condition. The “effective amount” can vary depending on the compound, the disease and its severity, and the age, weight, etc., of the subject to be treated.

### Germinants

In some embodiments, the germinant is a compound of Formula I:

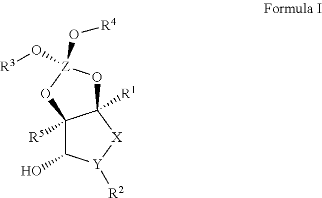

wherein R¹ is H, alkyl, cycloalkyl, OH, alkoxy, or halo; R² is H, alkyl, cycloalkyl, OH, or alkoxy; R³ and R⁴ are independently alkyl or H; R⁵ is alkyl or cycloalkyl; X is O, CH₂, or S; Y is CH or N; and Z is C; or salts thereof. In some embodiments, R¹ is H. In another embodiment R¹ is alkyl. In some embodiments, R¹ is C₁₋₂₂ alkyl. In other embodiments, R¹ is C₁₋₆ alkyl. In some other embodiments, R¹ is cycloalkyl. In some embodiments, R¹ is C₃₋₆ cycloalkyl. In some embodiments, R² is H. In another embodiment R² is alkyl. In some embodiments, R² is C₁₋₂₂ alkyl. In other embodiments, R² is C₁₋₆ alkyl. In some other embodiments, R² is cycloalkyl. In some embodiments, R² is C₃₋₆ cycloalkyl. In some embodiments, R³ and R⁴ are independently H or alkyl. In some embodiments, R³ is H. In other embodiments, R³ is alkyl. In some other embodiments, R³ is C₁₋₂₂ alkyl. In other embodiments, R³ is C₁₋₆ alkyl. In some embodiments, R⁴ is H. In other embodiments, R⁴ is alkyl. In some other embodiments, R⁴ is C₁₋₂₂ alkyl. In other embodiments, R⁴ is C₁₋₆ alkyl. In some embodiments, R⁵ is alkyl. In some embodiments, R⁵ is C₁₋₂₂ alkyl. In other embodiments, R⁵ is C₁₋₆ alkyl. In some other embodiments, R⁵ is cycloalkyl. In some embodiments, R⁵ is C₃₋₆ cycloalkyl. In some embodiments, X is O. In other embodiments, X is CH₂. In further embodiments, X is S. In some embodiments, Y is CH. In other embodiments, Y is N. In some embodiments, Z is C.

In some embodiments, the germinant is a compound of Formula I:

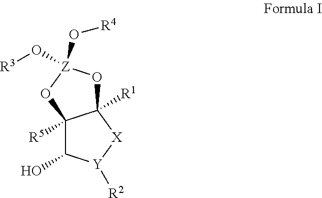

wherein R¹ is H, alkyl, cycloalkyl, OH, alkoxy, or halo; R² is H, alkyl, cycloalkyl, OH, or alkoxy; R³ and R⁴ are independently alkyl or H; R⁵ is alkyl or cycloalkyl; X is O, CH₂, or S; Y is CH or N; and Z is B; or salts thereof. In some embodiments, R¹ is H. In another embodiment R¹ is alkyl. In some embodiments, R¹ is C₁₋₂₂ alkyl. In other embodiments, R¹ is C₁₋₆ alkyl. In some other embodiments, R¹ is cycloalkyl. In some embodiments, R¹ is C₃₋₆ cycloalkyl. In some embodiments, R² is H. In another embodiment R² is alkyl. In some embodiments, R² is C₁₋₂₂ alkyl. In other embodiments, R² is C₁₋₆ alkyl. In some other embodiments, R² is cycloalkyl. In some embodiments, R² is C₃₋₆ cycloalkyl. In some embodiments, R³ and R⁴ are independently H or alkyl. In some embodiments, R³ is H. In other embodiments, R³ is alkyl. In some other embodiments, R³ is C₁₋₂₂ alkyl. In other embodiments, R³ is C₁₋₆ alkyl. In some embodiments, R⁴ is H. In other embodiments, R⁴ is alkyl. In some other embodiments, R⁴ is C₁₋₂₂ alkyl. In other embodiments, R⁴ is C₁₋₆ alkyl. In some embodiments, R⁵ is alkyl. In some embodiments, R⁵ is C₁₋₂₂ alkyl. In other embodiments, R⁵ is C₁₋₆ alkyl. In some other embodiments, R⁵ is cycloalkyl. In some embodiments, R⁵ is C₃₋₆ cycloalkyl. In some embodiments, X is O. In other embodiments, X is CH₂. In further embodiments, X is S. In some embodiments, Y is CH. In other embodiments, Y is N. In some embodiments, Z is B.

In further embodiments, the germinant is a compound of Formula II:

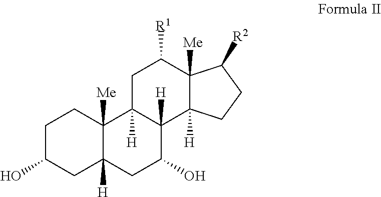

wherein R¹ is H, alkyl, OH, alkoxy, or halo; R² is OH, alkyl, alkoxy, halo, amino, aminoalkyl, aminodialkyl, alkylamino, alkylaminoalkyl, CO₂R⁶, COR⁶, or CON(R⁶)₂; wherein each R⁶ is independently H or alkyl; or has the structure of Formula IIa:

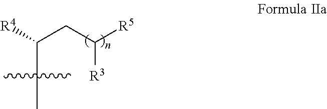

wherein each R³ is independently H, alkyl, alkoxy, CO₂R⁶, or halo; R⁴ is H, alkyl, OH, alkoxy, or halo; R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl; each R⁶ is independently H or alkyl; and n is 0, 1, 2, 3, 4, or 5; or has the structure of Formula IIb:

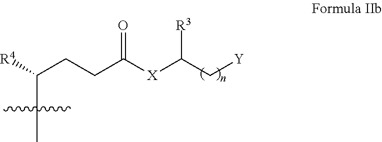

wherein R³ is H, alkyl, alkoxy, CO₂R⁶, or halo; R⁴ is H, alkyl, OH, alkoxy, or halo; X is NR⁶, O or C(R⁶)₂; Y is SO₃H, PO₃H, CO₂R⁶, CONR⁶, H, alkyl, OH, alkoxy, halo, aminoalkyl, or amino; and each R⁶ is independently H or alkyl; n is 0, 1, 2, 3, 4, or 5; or a salt thereof.

In some embodiments, germinants include, but are not limited to dodecylamine, CaDPA, KCl, Pi, taurocholate, glycine, L-phenylalanine, L-arginine, THMF and ppGppp.

In certain embodiments, the germinant is a selected from the group comprising
2,3,3,4(2H)-Furantetrol, dihydro-2-methyl-, (2R,4S)-
2,3,3,4(2/-I)-Furantetrol, dihydro-2-methyl-, (2S,4S)-
2,3,3,4(2H)-Furantetrol, dihydro-2-methyl-, (4S)-
2,3,3,4(2H)-Furantetrol, dihydro-2-(methyl-13C)-, (2S,4S)- (9CI)
2,3,3,4(2H)-Furantetrol, dihydro-2-(methyl-13C)-, (2R,4S)- (9CI)
2,3,3,4(2H)-Furantetrol, dihydro-2-(methyl-13C)-, (4S)-
2,3,3,4(2H)-Furantetrol-2-13C, dihydro-2-(methyl-13C)-, (4S)-
2,3,3 (2H)-Furantriol, dihydro-4-methoxy-2-methyl-
2,3,3(2H)-Furantriol, 4-ethoxydihydro-2-methyl
2,3,3 (2H)-Furantriol, dihydro-2-methyl-4-propoxy-
3-Furanol, tetrahydro-2,4,4,5-tetramethoxy-5-methyl-
2,3,3,4(2H)-Furantetrol, dihydro-2-(trifluoromethyl)-,(4S)-
2,3,3(2H)-Furantriol, 4-(hexyloxy)dihydro-2-methyl-
2,3,3(2H)-Furantriol, dihydro-2-methyl-4-(phenylmethoxy)-
Borate(1-), [[(2S,3S,4S)-dihydro-2-methyl-2,3,3,4(2H)-furantetrolato(2-)-KO3,KO4]dihydroxy-, (T-4)-
3,4-Furandiol, tetrahydro-2,5-dimethoxy-2-methyl- (7CI)
erythro-2-Hexulofuranose, 1,6-dideoxy-5-C-niethoxy-(5E1)-(9CI)
2-Hexulofuranoside, methyl 1,6-dideoxy-5-C-methoxy-, (9CI)
α-D-Psicofuranose, 5-C-hydroxy- (9CI)
3-D-Psicofuranose, 5-C-hydroxy- (9CI)
β-L-Tagatofuranose, 5-C-hydroxy- (9CI)
3,4-Furandiol, tetrahydro-2-(hydroxymethyl)-2,5-dimethoxy- (9CI)
3-Furanol, tetrahydro-2,4,4,5-tetramethoxy-5-methyl-2-Furoic acid, tetrahydro-3,4-dihydroxy-2,5-dimethoxy-(6CI)
2-Furaldehyde, tetrahydro-3,4-dihydroxy-2,5-dimethoxy-, dimethyl acetal (6CI)
3,4-Furandiol, tetrahydro-2,5-dimethoxy-2-methyl-, diacetate (7CI)
2-Furoic acid, tetrahydro-3,4-dihydroxy-2,5-dimethoxy-, methyl ester (6C1,7CI)
D-erythro-Pentofuranose, 5-deoxy-4-C-hydroxy-2,3-O-(1-methylethylidene)-, (4)-(9CI)
β-D-Fructofuranose, 5-C-hydroxy-, 1-(dihydrogen phosphate) (9CI)
2-Furoic acid, tetrahydro-3,4-dihydroxy-2,5-dimethoxy-5-methyl-, methyl ester (7CI)
2-Furamide, tetrahydro-3,4-dihydroxy-2,5-dimethoxy-(6CI)
Furfuryl alcohol, tetrahydro-3,4-dihydroxy-2,5-dimethoxy-, acetate (6CI)
α-D-Glucopyranoside, 4-C-hydroxy-α-D-arabinofuranosyl (9CI)
Pentofuranoside, methyl 5-deoxy-3-C-(dimethoxymethyl)-4-C-methoxy-(9CI)
D-erythro-Pentofuranoside, methyl 5-deoxy-4-C-hydroxy-2,3-O-(1-methylethylidene)-, (4)- (9CI)
D-erythro-Pentofuranoside, methyl 5-deoxy-4-C-methoxy-2,3-O-(1-methylethylidene)-, (4)- (9CI)
α-D-Fructofuranose, 5-C-hydroxy-, 1,6-bis(dihydrogen phosphate) (9CI)
β-D-Fructofuranose, 5-C-hydroxy-, 1,6-bis(dihydrogen phosphate) (9CI)
α-D-ribo-Hexopyranosid-3-ulose, (4R)-4-C-hydroxy-β-D-arabinofuranosyl (9CI)
α-D-Galactopyranoside, (5S)-5-C-hydroxy-β-D-threo-2-pentulofuranosyl (9CI)
α-D-Glucopyranoside, (5S)-5-C-hydroxy-β-D-threo-2-pentulofuranosyl (9CI)
2,7,12,13-Tetraoxatricyclo[7.2.1.1^{3,6}]tridecane-4,5,9,10,11-pentol, 3-(hydroxymethyl)-1,6-dimethyl-, (4S,5R,10S,11R)-
β-D-Xylofuranose, 1,5-anhydro-4-C-(α-D-glucopyranosyloxy)- (9CI)
2-Furoic acid, tetrahydro-3,4-dihydroxy-2,5-dimethoxy-, -methyl ester, diacetate (7CI)
β-D-threo-2,5-Hexodiulo-2,6-pyranose, 5-hydrate, 5,2¹:5,3¹-dianhydride with 5-C-hydroxy-α-L-sorbofuranose (9CI)
D-erythro-Pentofuranose, 5-bromo-5-deoxy-4-C-hydroxy-2,3-O-(1-methylethylidene)-, (4)-
D-erythro-Pentofuranose, 5-deoxy-4-C-hydroxy-5-iodo-2,3-O-(1-methylethylidene)-, (4)-
3,4-Furandiol, 2-[(benzoyloxy)methyl]tetrahydro-2,5-dimethoxy-(9CI)
β-D-ribo-Pentofuranoside, methyl 5-bromo-5-deoxy-4-C-hydroxy-2,3,O-(1-methylethylidene)-
α-L-lyxo-Pentofuranoside, methyl 5-bromo-5-deoxy-4-C-hydroxy-2,3-O-(1-methylethylidene)-
α-D-ribo-Pentofuranoside, methyl 5-bromo-5-deoxy-4-C-hydroxy-2,3-O-(1-methylethylidene)-
3-L-lyxo-Pentofuranoside, methyl 5-bromo-5-deoxy-4-C-hydroxy-2,3-O-(1-methylethylidene)-
D-erythro-Pentofuranoside, methyl 5-bromo-5-deoxy-4-C-hydroxy-2,3-O-(1-methylethylidene)-, (4)-
D-erythro-Pentofuranoside, methyl 5-deoxy-4-C-hydroxy-5-iodo-2,3-O-(1-methylethylidene)-, (4)-
Pentofuranoside, methyl 5-deoxy-3-C-(dimethoxymethyl)-4-C-methoxy-2,3-bis-O-(trimethylsilyl)- (9CI)
Furo[3,4-d]-1,3,2-dioxaphosphol-4-ol, tetrahydro-2,6-dimethoxy-4,6-dimethyl-, 2-oxide (9CI)
D-erythro-L-ribo-5-Nonuto-5,2-furanose, 5,9-anhydro-1,6,7,8-tetradeoxy-3-O-[(1,1-dimethylethyl)dimethylsilyl]2-C-hydroxy-6,8-dimethyl-4-O-methyl-, (5S)- (9CI)
Pentofuranoside, methyl 5-deoxy-3-C-(dimethoxymethyl)-4-C-methoxy-, diacetate (9CI)
13,14-Dioxatricyclo[8.2.1.1^{4,7}]tetradecane-5,6,11,12-tetrol, 1,4,7,10-tetramethoxy-
3,4-Furandiol, tetrahydro-2,5-dimethoxy-2-methyl-, dibenzoate (7CI)
β-D-Ribofuranoside, (2-nitrophenyl)methyl 4-C-methoxy-
3-L-erythro-Hexofuranosid-5-ulose, methyl 6-deoxy-4-C-methoxy-2,3-O-(1-methylethylidene)-, (S)- (9CI)
α-D-Lyxofuranose, 4-C-ethoxy-5-O-(phenylmethyl)-, triacetate (9CI)
β-D-Lyxofuranose, 4-C-ethoxy-5-O-(phenylmethyl)-, triacetate (9CI)
β-D-Ribofuranoside, (2-nitrophenyl)methyl 4-C-methoxy-2-O-methyl-
α-D-Glucofuranose, 4-O-methyl-1,2:5,6-bis-O-(1-methylethylidene)-2,3,7-Trioxabicyclo[2.2.1]heptane-5,6-diol, 1,4-dimethyl-, dinitrate, (5-endo, 6-exo)- (9CI)
Galactitol, 2,5-anhydro-1,6-dideoxy-2,5-dimethoxy-3,4-di-C-methyl-, cyclic 3,4-(hydrogen phosphate) (8CI)
a-D-Tagatofuranoside, methyl 1,6-dideoxy-5-C-methoxy-3,4-di-C-methyl-, cyclic hydrogen phosphate (9CI)
β-D-Riboruranoside, methyl 4-C-(1-cyanoethoxy)-5-deoxy-2,3-O-(1-methylethylidene)- (9CI)
Phosphoric acid, methyl ester, cyclic 3,4-ester with tetrahydro-5-methoxy-2,3,4,5-tetramethyl-2,3,4-furantriol (7CI)
α-L-Tagatofuranose, 2,5-anhydro-1,6-dideoxy-5-C-methoxy-3,4-di-C-methyl-, cyclic 3,4-(methyl phosphate), (R)-(9CI)
α-L-Tagatofuranose, 1,6-dideoxy-5-C-methoxy-3,4-di-C-methyl-, cyclic 3,4-(methyl phosphate), (S)- (9CI)
Fructofuranose, O-α-D-galactopyranosyl-(1→6)-O-α-D-glucopyranosyl-6-t-(1→4)-, β-D- (8CI)
Raffinose-6′-t (8CI)
SaH-Oxireno[8,8a]naphtho[2,3-b]furan-5a,6-diol, decahydro-7,8a-dimethoxy-4,4a,6-trimethyl-, (1aR,4S,4aR,5aR,6S,7S,8aS,9aS)-
Molybdate(1-), [μ-[1,6-dideoxy-5-C-hydroxy-3,4-di-C-methyl-α-psicofuranosato(3-)-O²,O³:O³,O⁴]]-μ-oxotetraoxodi-, steroisomer (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-) (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-), compd. with 1,1′-oxybis[ethane] (2:1) (9CI)
9a H-4a,8-Epoxy-1,3-dioxolo[4,5]furo[2,3-d]oxepin-9a-ol, hexahydro-2,2,8-trimethyl-, [3aS-(3aα,4aα,8α,9aβ,9bα)]-(9CI)
α-Lyxofuranoside, methyl 3-C-[(benzoyloxy)methyl]-5-deoxy-4-C-methoxy-, 2-acetate (9CI)
2-Furoic acid, tetrahydro-3,4-dihydroxy-2,5-dimethoxy-, methyl ester, dibenzoate (7CI)
β-D-ribo-Heptofuranoside, methyl 4,6-anhydro-5,7-dideoxy-4-C-hydroxy-6-Cmethyl-2,3-O-(1-methylethylidene)- (9CI)
β-L-Sorbofuranose, 5-C-hydroxy-1,3:4,6-bis-O-(phenylmethylene)-, [1(R),4(R)]- (9CI)
β-L-Sorbofuranose, 5-C-hydroxy-1,3:4,6-bis-O-(phenylmethylene)-(9CI)
α-D-Glucofuranose, 4-O-methyl-1,2:5,6-bis-O-(1-methylethylidene)-, 3-acetate
α-D-Galactofuranose, 4-O-methyl-1,2:5,6-bis-O-(1-methylethylidene)-, 3-acetate
2,4,6-Metheno-2H-cyclopenta[g]furo[2,3,4-ij][2]benzopyran-2,5a,6a,9a,9b,9c(2aH,6H,7H)-hexol, tetrahydro-2a,6,9-trimethyl-4-(1-methylethyl)-, (2S,2aS,4S,5aR,6S,6aS,9R,9aS,9bR,9cS,10S)- (9CI)
Furo[2,3-d:4,5-d]bis[1,3]dioxole, tetrahydro-2,2,3a,6,6,7b-hexamethyl-, (3aα,4aα,7aβ,7bβ)- (9CI)
1,4a-(Epoxymethano)-4aH-xanthen-9(2H)-one, 1,3,4,9a-tetrahydro-1,9a-dihydroxy-11-methoxy-
α-L-Sorbofuranose, 5-C-methoxy-1,3:4,6-bis-O-(phenylmethylene)-, [1(R),4(R),5S]- (9CI)
Furo[2,3-d:4,5-d]bis[1,3]dioxole, tetrahydro-2,2,3a,4a,6,6-hexamethyl-, (3aα,4aβ,7aβ,7bα)- (9CI)
Spiro[furan-2(3H),4′(3′aH)-furo[3,4-d][1,3]dioxole], 6′,6′a-dihydro-6′-methoxy-2′,2′-dimethyl-, (2R,3′aS,6′R,6′aR)-
Octofuranosiduronic acid, methyl 3,6-anhydro-5-deoxy-4-C-methoxy-6-C-(methoxycarbonyl)-, methyl ester (9CI)
3-L-erythro-Hexofuranosid-5-ulose, methyl 6-deoxy-4-C-methoxy-2,3-O-(1-methylethylidene)-, oxime, (4R)- (9CI)
2-Propanone, 1-hydroxy-1-[tetrahydro-6-hydroxy-2,3a,5-trimethyl-5,2-(epoxymethano)furo[2,3-d]-1,3-dioxol-8-yl]-, [2R-[2α,3aμ,5α,6β,6aβ,8S*(R*)]]- (9CI)
β-L-erythro-β-L-lyxo-Decofuranos-7-ulo-7,10-furanose, 10-C-(acetyloxy)-3,7-anhydro-5,6-dideoxy-, tetraacetate, (10R)- (9CI)
Inulobiose, octaacetate (5CI)
2,3,7-Trioxabicyclo[2.2.1]heptane-1-acetonitrile, 5-[[(1,1-dimethylethyl)dimethylsilyl]oxy]-6-methoxy-4-methyl-, (1R,4S,5R,6R)-
D-gluco-Nonitol, 2,5-anhydro-1,6,7,8-tetradeoxy-3-O-[(1,1-dimethylethyl)dimethylsilyl]-2,5-C-epidioxy-6,8-dimethyl-4-O-methyl-, (2,5)- (9CI)
β-D-Lyxofuranose, 4-C-ethoxy-2,3-O-(1-methylethylidene)-5-O-(phenylmethyl)-, acetate (9CI)
2-Heptenal, 6-[(5S,6R)-5-hydroxy-6-methoxy-4-methyl-2,3,7-trioxabicyclo[2.2.1]hept-1-yl]-4-methyl-, (2E,4S,6R)-
Spiro[1,2-dioxin-3(6H),5′(3′aH)-furo[2,3-d][1,3]dioxole]-6,6′-diol, 6′,6′a-dihydro-2′,2′,6-trimethyl-, 6′-acetate, (3R,3′aS,6′R,6′aR)-
Spiro[1,2-dioxin-3(6H), 5′(3′aH)-furo[2,3-d][1,3]dioxole]-6,6′-diol, 6′,6′a-dihydro-2′,2′,6-trimethyl-, 6′-acetate, (3S,3′aS,6′R,6′aR)-
5,8-Epoxy-1,4-dioxino[2,3-d][1,2]dioxin, hexahydro-4a,5,8,8a-tetramethyl-, (4aR,5S,8R,8aS)-rel-
5,8-Epoxy-1,4-dioxino[2,3-d][1,2]dioxin, hexahydro-4a,5,8,8a-tetramethyl-, (4aR,5R,8S,8aS)-rel-
5,8-Epoxy-1,4-dioxino[2,3-d][1,2]dioxin, hexahydro-4a,5,8,8a-tetramethyl-
4H-1-Benzopyran-4-one, 2-(3,4-dihydroxyphenyl)-5,6-dihydroxy-7-[(4-C-hydroxy-α-D-ribofuranosyl)oxy]- (9CI)
Furo[2,3-d:4,5-d]bis[1,3]dioxole, tetrahydro-2,2,3a,4a,6,6,7a,7b-octamethyl-, (3aα,4aα,17aβ,7bβ)- (9CI)
6,9-Epoxy-2H-o-dioxino[4,5-b][1,4]dioxepin, hexahydro-5a,6,9,9a-tetramethyl-, stereoisomer (8CI)
6,9-Epoxy-2H-o-dioxino[4,5-b][1,4]dioxepin, hexahydro-5a,6,9,9a-tetramethyl-, stereoisomer (8CI)
L-gulo-Nonose, 5,8-anhydro-2,3,4,9-tetradeoxy-7-O-[(1,1-dimethylethyl)dimethylsilyl]-5,8-C-epidioxy-2,4-dimethyl-6-O-methyl-, (5,8)- (9CI)
α-L-Sorbofuranose, 5-C-methoxy-1,3:4,6-bis-O-(phenylmethylene)-, acetate, [1(R),4(R),5S]- (9CI)
Spiro[1,2-dioxin-3(6H),5′ (3′aH)-furo[2,3-d)[1,3]dioxol]-6-ol, 6′,6′a-dihydro-6′-methoxy-6-methyl-2′-(trichloromethyl)-, (2′S,3S,3′aS,6′R,6′aR)-
Spiro[1,2-dioxin-3(6H),5′(3′aH)-furo[2,3-d)[1,3]dioxol]-6-ol, 6′,6′a-dihydro-6′-methoxy-6-methyl-2′-(trichloromethyl)-, (2′R,3R,3′aS,6′R,6′aR)-
7,10-Epoxy[1,2]dioxino[4,5-b][1,4]dioxocin, octahydro-6a,7,10,10a-tetramethyl-
Furo[2,3-d:4,5-d]bis[1,3]dioxole-3a(4aH)-carboxylic acid, dihydro-2,2,6,6-tetramethyl- (9CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, (3aR,3′aS,3bR,5S,6′S,6′aR,7aS,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, (3aR,3′aS,3bR,5S,6′S,6′aR,7aR,8aR)- (9CI)
Spiro[1,2-dioxin-3 (6H),5′(3′aH)-furo[2,3-d][1,3]dioxole]-6,6′-diol, 6′,6′a-dihydro-2′,2′,6-trimethyl-, 6,6′-diacetate, (3S,3′aS,6′R,6′aR)-
Spiro[1,2-dioxin-3 (6H),5′(3′aH)-furo[2,3-d][1,3]dioxole]-6,6′-diol, 6′,6′a-dihydro-2′,2′,6-trimethyl-, 6,6′-diacetate, (3R,3′aS,6′R,6′aR)-
2H-Naphtho[1,2-b]pyran-2-one, 6-[[5-C-(α-D-glucopyranosyloxy)-(3-D-fructofuranosyl]oxy]-7-hydroxy-3-methyl-
Spiro[furo[3,4-d]-1,3-dioxole-4(3aH),5′(4′H)-isoxazole], 6,6a-dihydro-6-methoxy-2,2,3′-trimethyl-, (3aS,4R,6R,6aR)-
Spiro[1,2-dioxin-3 (6H),5′(3′aH)-furo[2,3-d][1,3]dioxole]-6,6′-diol, 6′,6′a-dihydro-6-methyl-2′-(trichloromethyl)-, 6′-acetate, (2′S,3S,3′aS,6′R,6′aR)-
Spiro[1,2-dioxin-3(6H), 5′(3′aH)-furo[2,3-d][1,3]dioxole]-6,6′-diol, 6′,6′a-dihydro-6-methyl-2′-(trichloromethyl)-, 6′-acetate, (2′R,3R,3′aS,6′R,6′aR)-
α-D-Glucofuranose, 4-C-hydroxy-1,2:5,6-bis-O-(1-methylethylidene)-, 3-(3-chlorobenzoate)
Spiro[5H-1,3-dioxolo[4,5]furo[3,2-b]pyran-5,5′(3′aH)-furo[2,3-d][1,3]dioxol]-6′-ol, 3a,6,6′,6′a,7,8a-hexahydro-2,2,2′,2′-tetramethyl-, (3aR,3′aS,5S,6′S,6′aR,8aR)-
Spiro[furo[3,4-d]-1,3-dioxole-4(3aH),5′(4′H)-isoxazole], 3′-ethyl-6,6a-dihydro-6-methoxy-2,2-dimethyl-, (3aS,4R,6R,6aR)-
1,4-Epoxy-o-dioxino[4,5-b][1,4]benzodioxin, 1,4,4a, 10a-tetrahydro-1,4,4a, 10a-tetramethyl-, stereoisomer (8CI)
1,4-Epoxy-o-dioxino[4,5-b][1,4]benzodioxin, 1,4,4a, 10a-tetrahydro-1,4,4a, 10a-tetramethyl-, stereoisomer (8CI)
1,4:6,9-Diepoxy-p-dioxino[2,3-d:5,6-d]bis-o-dioxin, octahydro-1,4,4a,5a,6,9,9a,10a-octamethyl-, stereoisomer (8CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2′,2′-tetramethyl-, (3aR,3′aS,3bR,5S,6′S,6′aR,7aS,8aR)- (9CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2,2′-tetramethyl-, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, 6′-acetate, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, 6′-(1-aminoethyl)-3a,3b,6,6′,6′a,7,7a,8a-octahydro-2,2,2′,2′-tetramethyl-, (3aR,3′aS,3bS,5S,6′S,6′aR,7aR,8aR)-
L-glycero-β-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-β-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
β-L-threo-Pentofuranose, 4-C-(acetyloxy)-5-deoxy-5-fluoro-1,2-O-(1-methylethylidene)-, 4-methylbenzenesulfonate, (4ξ)- (9CI)
α-D-Glucopyranoside, methyl 6-deoxy-4-O-(4-C-hydroxy-2,3,5-tri-O-methyl-α-D-arabinofuranosyl)-2,3-di-O-methyl-6-[[(4-nitrophenyl) sulfonyl]amino]-
Spiro[furo[3,4-d]-1,3-dioxole-4(3aH),5′(4′H)-isoxazole], 6,6a-dihydro-6-methoxy-2,2-dimethyl-3′-[(3 aR,4R,6R,6aR)-tetrahydro-6-methoxy-2,2-dimethylfuro[3,4-d]-1,3-dioxol-4-yl]-, (3aS,4R,6R,6aR)
Spiro[furo[3,4-d]-1,3-dioxole-4(3aH),2′(3′H)-pyrrolo[1,2-b]isoxazole], hexahydro-6-methoxy-4′,5′-bis(methoxymethoxy)-2,2-dimethyl-, (2′R,3aS,3′aS,4′S,5′S,6R,6aR)-
5,2,9-Ethanylylidene-1-benzoxepin-8,11(2H)-dione, 4-[(2R,3R)-3,4-dihydro-3,5,7-trihydroxy-2H-1-benzopyran-2-yl]-6-[(2S,3S)-3,4-dihydro-3,5,7-trihydroxy-2H-1-benzopyran-2-yl]-5,5a,9,9a-tetrahydro-2,9,9a,10-tetrahydroxy-, (2R,5S,5aS,9S,9aS,10R)- (9CI)
Spiro[5H-1,3-dioxolo[4,5]furo[3,2-b]pyran-5,5′(3′a H)-furo[2,3-d][1,3]dioxol]-6′-ol, 3a,6,6′,6′a,7,8a-hexahydro-2,2,2′,2′-tetramethyl-, 6′-acetate, (3aR,3′aS,5S,6′S,6′aR,8aR)-
Spiro[furo[3,4-d)-1,3-dioxole-4(3aH),5′(4′H)-isoxazole], 6,6a-dihydro-6-methoxy-2,2-dimethyl-3′-phenyl-, (3aS,4R,6R,6aR)-
1,4-Epoxycyclobuta[5,6]-p-dioxino[2,3-d)-o-dioxin, 1,4,4a,5a,7a,8a-hexahydro-1,4,4a,5a,6,7,7a,8a-octamethyl-, stereoisomer (8CI)
1,4-Epoxycyclobuta[5,6]-p-dioxino[2,3-d)-o-dioxin, 1,4,4a,5a,7a,8a-hexahydro-1,4,4a,5a,6,7,7a,8a-octamethyl-, stereoisomer (8CI)
Spiro[furo[3,4-d)-1,3-dioxole-4-(3aH),6′-[6H]1,2]oxazine]-3′-carboxylic acid, hexahydro-6-methoxy-2,2-dimethyl-, ethyl ester, (3′ R,3a5,4R,6R,6aR)-
Spiro[furo[3,4-d)-1,3-dioxole-4(3aH),5′-isoxazolidine], dihydro-6-methoxy-2,2-dimethyl-2′-(phenylmethyl)-, (3aS,4R,6R,6aR)-
2H-Naphtho[1,2-b]pyran-2-one, 7-(acetyloxy)-3-methyl-6-[[1,3,4,6-tetra-O-acetyl-5-C-[(2,3,4,6-tetra-O-acetyl-α-D-glucopyranosyl)oxy]-β-D-fructofuranosyl]oxy]-
Spiro[furo[3,4-d)-1,3-dioxole-4(3aH),5′(4′H)-isoxazole), 6,6a-dihydro-6-methoxy-2,2-dimethyl-3′-(4-methylphenyl)-, (3aS,4R,6R,6aR)-
3,6-Epoxy-2H,8H-pyrimido[6,1-b][1,3]-oxazocine-8,10(9H)-(dione, 3,4,5,6-tetrahydro-4-hydroxy-, acetate (ester)(7CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, 6′-benzoate, (3aR,3′aS,3bS,5S,6′S,6′aR,7aR,8aR)-
Spiro[5H-1,3-dioxolo[4,5][furo[3,2-b]pyran-5,5′(3′aH)-furo[2,3-d) [1,3]dioxol]-6′-ol, octahydro-2,2,2′,2′-tetramethyl-6′-(nitromethyl)-, (3aR,3′aS,3bS,5S,6′S,6′aR,7aR,8aR)-
Spiro[furo[3,4-d)-1,3-dioxole-4(3aH),6′-[6H]1,2]oxazine]-3′-carboxylic acid, 4′,5′,6,6a-tetrahydro-6-methoxy-2,2-dimethyl-, ethyl ester, (3aS,4R,6R,6aR)-
Hexanoic acid, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Acetamide, N-[1-[(2′R,3′R,3aS,3′aS,5′S,6S,6aR,7′aR)-2′,3′-bis(acetyloxy)-2′,3′,3′a,6,6′,6a,7′,7′a-octahydro-6-hydroxy-2,2-dimethylspiro[furo[2,3-d]-1,3-dioxole-5(3aH),5′-[5H]furo[3,2-b]pyran]-6-yl]ethyl]-
Spiro[5H-1,3-dioxolo[4,5]furo[3,2-b]pyran-5,5′(3′aH)-furo[2,3-d)[1,3]dioxol]-6′-ol, 3a,6,6′,6′a,7,8a-hexahydro-2,2,2′,2′-tetramethyl-6′-(nitromethyl)-, (3aR,3′aS,5S,6′S,6′aR,8aR)-
Spiro[5H-1,3-dioxolo[4,5]furo[3,2-b]pyran-5,5′(3′aH)furo[2,3-d)[1,3]dioxol]-6′-ol, 3a,6,6′,6′a,7,8a-hexahydro-2,2,2′,2′-tetramethyl-, 6′-benzoate, (3aR,3′aS,5S,6′S,6′aR,8aR)-
Spiro[furo[3,4-d)-1,3-dioxole-4(3 aH),5′(4′H)-isoxazole], 3′-(2,6-dichlorophenyl)-6,6a-dihydro-6-methoxy-2,2-dimethyl-, (3aS,4R,6R,6aR)-
1,6,11,14,16,20,23,24-Octaoxahexaspiro[3.0.3.0.0.4.0.3.0.3.1.1]tetracosane
Hexanoic acid, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3)dioxol]-6′-yl ester
Benzoic acid, 4-chloro-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)furo[2,3-d[[1,3]dioxol]-6′-yl ester
Benzoic acid, 4-bromo-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)furo[2,3-d][1,3]dioxol]-6′-yl ester
Spiro[5H-1,3-dioxolo[4,5]furo[3,2-b]pyran-5,5′(3′aH)-furo[2,3-d][1,3]dioxol]-6′-ol, 3a6,6′,6′a,7,8a-hexahydro-2,2,2′,2′-tetramethyl-6′-(1-nitroethyl)-, (3′aS3aR,5S,6′S,6′aR,8aR)-
Sorbofuranose, 2,3:4,6-di-O-isopropylidene-, p-toluenesulfonate, α-L-(7CI)
Benzoic acid, 4-chloro-, (3aR,3′aS,5S,6′S,6′aR,8aR)-3a,6,6′,6′a,7,8a-hexahydro-2,2,2,2′-tetramethylspiro[5H-1,3-dioxolo[4,5]furo[3,2-b]pyran-5,5′(3′aH)-furo[2,3-d][1,3]dioxol]-6′-yl ester
Benzoic acid, 4-bromo-, (3aR,3′aS,5S,6′S,6′aR,8aR)-3a,6,6′,6′a,7,8a-hexahydro-2,2,2,2′-tetramethylspiro[5H-1,3-dioxolo[4,5]furo[3,2-b]pyran-5,5′(3′aH)-furo[2,3-d][1,3]dioxol]-6′-yl ester
Spiro[furo[2,3-d]-1,3-dioxole-5(3aH),6′-[6H]1,2]oxazine]-3′-carboxylic acid, hexahydro-2,2-dimethyl-6-(phenylmethoxy)-, ethyl ester, (3′R,3aS,5R,6R,6aR)-
Spiro[furo[3,4-d]-1,3-dioxole-4(3aH),6′-[6H-1,2]oxazine]-2′,3′(3′H)-dicarboxylic acid, tetrahydro-6-methoxy-2,2-dimethyl-, 2′-(1,1-dimethylethyl) 3′-ethyl ester, (3′R,3aS,4R,6R,6aR)-
Spiro[furo[2,3-d]-1,3-dioxole-5(3 aH),6′-[6H]1,2]oxazine]-3′-carboxylic acid, 4′,5′,6,6a-tetrahydro-2,2-dimethyl-6-(phenylmethoxy)-, ethyl ester, (3aS,5R,6R,6aR)-
β-D-Galactofuranose, 3,4-O-[(acetylamino)phenylmethylene]-4-C-hydroxy-, 1-acetate 2,5,6-tribenzoate (9CI)
β-D-Galactofuranose, 3,4-O-[(2,5-dioxo-1-pyrrolidinyl)phenylmethylene]-4-C-hydroxy-, 1-acetate 2,5,6-tribenzoate (9CI)
Osmium, tetraoxotetrakis(pyridine)[μ-[1-(tetrahydro-2,3,4,5-tetrahydroxy-2-furanyl) ethanonato(4-)]]di-, stereoisomer (9CI)
β-D-Galactofuranose, 3,4-O-[(1,3-dihydro-1,3-dioxo-2H-isoindol-2-yl)phenylmethylene]-4-C-hydroxy-, 1-acetate 2,5,6-tribenzoate (9CI)
Piperazinone, 4-[(dihydro-2,2,6,6-tetramethylfuro[2,3-d;4,5-d]bis[1,3]dioxol-3a(4aH)-yl) carbonyl]-6-ethyl-1-[(4-methylphenyl)methyl]- (9CI)
Quinoline, 5,7-dichloro-1-[(dihydro-2,2,6,6-tetramethylfuro[2,3-d:4,5-d]bis[1,3]dioxol-3a(4aH)-yl) carbonyl]-4-(4-fluorophenoxy)-1,2-dihydro-(9CI)

In some embodiments, the bacterial endospore germinating film comprises a germinant that modulates one or more proteins selected from the group that comprises cwlJ, sleB, cwlD, spoVAC, spoVAD, spoVAE, SecA, LsrB, RelA, SpoT, DksA, cell wall hydrolase, germination protease, probable germination-specific protease, N-acetylmuramoyl-L-alanine amidase, subtilisin-like serine germination related protease, germination-specific N-acetylmuramoyl-L-alanine amidase (autolysin), putative spore cortex-lytic hydrolase, putative-germination-specific protease, putative spore cortex-lytic enzyme, putative germination-specific protease, germination-specific N-acetylmuramoyl-L-alanine amidase, or a combination thereof. In certain embodiments, the bacterial endospore germinating film comprises a germinant that modulates one or more proteins selected from the group that comprises spoVAC, spoVAD, spoVAE, SecA, SpoT and RelA. In some embodiments, the germinant modulates spoVAC. In some embodiments, the germinant modulates spoVAD. In some embodiments, the germinant modulates spoVAE. In some embodiments, the germinant modulates SpoT. In some embodiments, the germinant modulates SecA. In some embodiments, the germinant modulates RelA.

### Nanoscale Particles

Some embodiments of the present disclosure describe a formulation or composition that comprises at least one nanoscale particle. In some embodiments, the nanoscale particles comprise silver, titanium, zinc, aluminum, iron, copper, platinum, zirconium, palladium, gold, manganese, mercury, magnesium, silica, chromium, cobalt, nickel, molybdenum, ruthenium, rhodium, cadmium, cesium, iridium, osmium, tungsten, selenium, antimony, tin, cerium, yttrium, samarium, lanthanum, gallium, erbium, bismuth, strontium, barium, arsenic, salt thereof, or combinations thereof. In certain specific embodiments, the nanoparticle comprises zinc oxide, copper oxide, iron oxide, aluminum oxide, platinum oxide, zirconium oxide, silicon oxide, tungsten oxide, silver oxide yttrium oxide, colloidal gold, an ionic silver salt, elemental silver, titanium dioxide, bismuth pyrithione, zinc pyrithione, zinc percarbonates, zinc perborates, bismuth salts, arsenic trioxide, arsenicals, or combinations thereof.

In some embodiments, the nanoparticle comprises colloidal silver, metallic silver, silver chloride, silver bromide, silver phosphate, silver nitrate, silver citrate, silver acetate, silver benzoate, silver pyrithione, or combinations thereof. In some embodiments, silver nanoparticles have a particle size of about 1 to about 100 nm or 5 and 200 nm are generally understood to slowly release antimicrobial silver ions (e.g., Ag⁺).

In some embodiments, the release rate of metal ions (e.g., silver ions) depends on the initial concentration and size of the nanoscale particles. In certain embodiments, the release rate of metal ions (e.g., silver ions) is an indicator of the biocidal activity of the nanoscale particles. For example, in some instances, silver nanoscale particles in an aqueous environment oxidize in the presence of oxygen and protons according to the stoichiometric reaction:

$${Ag}_{( s )} + \frac{1}{2} {O}_{2} + 2 {H}_{( aq )}^{+} ↔ 2 {Ag}_{( aq )}^{+} + {H}_{2} {O}_{( l )}$$

releasing Ag+ ions during particle dissolution.

In further or alternative embodiments, the nanoparticle comprises titanium dioxide in the anatase phase. In some embodiments, the nanoscale titanium dioxide is photocatalytically active. Titanium dioxide in the anatase phase is generally understood to promote oxidation-reduction (redox) reactions when irradiated with ultraviolet or visible light. In some embodiments, a nanoparticle containing titanium dioxide that is irradiated with visible or ultraviolet light in an aqueous environment (e.g., within a microorganism) and in certain situations produce hydroxyl ions (OH⁻), superoxide ions (O₂⁻), and/or hydrogen peroxide (H₂O₂). In additional or further embodiments, a nanoscale particle containing titanium dioxide that is exposed to visible light while in a cell or in contact with a cell produces a toxic environment and damages or kills the cell. In some embodiments, the titanium dioxide oxidizes organic material (e.g. microbes).

In certain embodiments, suitable copper nanoscale particles include cupric oxide, cuprous oxide, cuprous iodide, cupric iodide, cupric phosphate, copper (II) hydrogen phosphate, and cupric silicate.

In some embodiments, the nanoscale particles have an average particle size between 0.1 and 500 nm, 0.1 and 400 nm, 0.1 and 300 nm, 0.1 and 250 nm, 0.1 and 200 nm, 0.1 and 100 nm, 0.1 and 90 nm, 1 and 1000 nm, 1 and 500 nm, 1 and 450 nm, 1 and 400 nm, 1 and 350 nm, 1 and 300 nm, 1 and 250 nm, 1 and 225 nm, 1 and 200 nm, 1 and 175 nm, 1 and 150 nm, 1 and 125 nm, 1 and 100 nm, 1 and 75 nm, 1 and 50 nm, 1 and 40 nm, 1 and 30 nm, 1 and 25 nm, 1 and 20 nm, 1 and 15 nm, or 1 and 10 nm. In some embodiments, the nanoscale particles have an average particle size between 5 and 10 nm, 5 and 30 nm 10 and 250 nm, 10 and 200 nm, 50 and 200 nm, or 100 and 200 nm. In further or additional embodiments, the nanoscale particles have an average particle size of less than about 500 nm, less than about 450 nm, less than about 400 nm, less than about 350 nm, less than about 300 nm, less than about 250 nm, less than about 200 nm, less than about 175 nm, less than about 150 nm, less than about 125 nm, less than about 100 nm, less than about 95 nm, less than about 90 nm, less than about 85 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 28 nm, less than about 25 nm, less than about 23 nm, less than about 20 nm, less than about 18 nm, less than about 15 nm, or less than about 10 nm. In further or additional embodiments, the nanoscale particles have an average particle size of about 500 nm, about 400 nm, about 350 nm, about 300 nm, about 250 nm, about 200 nm, about 175 nm, about 150 nm, about 125 nm, about 100 nm, about 75 nm, about 60 nm, about 50 nm, about 25 nm, or about 10 nm. In some embodiments, the formulation comprises a bimodal distribution of particle sizes. In some embodiments, the formulation comprises a polydisperse population of particle sizes.

In some instances, for cases with similar silver concentrations by mass, samples with smaller particle size have a larger number concentration. In some instances, the increase in concentration of silver nanocrystals leads to a higher nanoparticle:bacterium ratio and a greater amount of available nanoparticle surface area.

In some embodiments, the rate of release of Ag+ ions is dependent upon the size of the nanocrystal, with smaller particles dissolving more readily. In some instances, the rate of release of nanoscale ions (e.g., silver nanoscale ions) is inversely proportional to the size of the nanoscale particle (e.g., nanoscale colloidal silver).

Some embodiments provided herein describe nanoscale particles having a diversity of shapes. In some embodiments, the nanoscale particles are spherical in shape. In other embodiments, the nanoscale particles are round plates, triangular plates, square plates, or hexagonal plates. In certain embodiments, the nanoscale particles are triangular plates. In some embodiments, the nanoscale particles are nanorods, hexagonal-shaped, cube-shaped, polyhedron-shaped, or star-shaped. In some embodiments, the formulation comprises a bimodal distribution of particle shapes. In some embodiments, the formulation comprises a polydisperse population of particle shapes.

In some embodiments, the nanoscale particles comprise composites. Non limiting examples of suitable nanoscale particles include alloy of silver containing about 2.5 wt % copper, alumina-silver nanoscale composite, titania-silver nanoscale composite, silver-copper nanoscale composite, silver-iron oxide nanoscale composite, silver-silica nanoscale composite, and silver-selenium nanoscale composite. In some embodiments, the formulation comprises a nanoparticle/polymer composite film (e.g., silver nanoparticle/polyvinylpyrrolidone (PVP), silver nanoparticle/polyvinyl alcohol (PVA), etc.)

Some embodiments provided herein describe one or more nanoscale particles optionally comprise a capping ligand or stabilizing agent. Non-limiting examples of capping or stabilizing agents include polyvinylpyrrolidone (PVP), polyethyleneimine, citrate, keratin, tannic acid, bovine serum albumin (BSA), ionic surfactants (e.g, sodium dodecyl sulfate (SDS)), non-ionic surfactants (e.g., Tween 80), linoleic acid, poly(methylvinylether-co-maleic anhydride (PVM/MA), and sophorolipid.

In some embodiments, the nanoscale particle has antimicrobial activity. In some embodiments, the nanoscale particle is attached to a spore. In further or additional embodiments, the nanoscale particle is an active agent. In yet further embodiments, the nanoscale particle kill the microbe after germination. In still further or additional embodiments, the nanoscale particle is an odor control agent, antimicrobial surface coating, self-cleaning surface coating, germicide, antibacterial agent, anti-microbial, anti-fungal, anti-viral agent, anti-protozoal agent, microbiostat, or disinfectant.

### Microscale Particles

Some embodiments of the present disclosure describe a formulation or composition comprises at least one microscale particle. In some embodiments, the microscale particles comprise silver, titanium, zinc, aluminum, iron, copper, platinum, zirconium, palladium, gold, manganese, mercury, magnesium, silica, chromium, cobalt, nickel, molybdenum, ruthenium, rhodium, cadmium, cesium, iridium, osmium, tungsten, selenium, antimony, tin, cerium, yttrium, samarium, lanthanum, gallium, erbium, bismuth, strontium, barium, arsenic, salt thereof, or combinations thereof. In certain specific embodiments, the microparticle comprises zinc oxide, copper oxide, iron oxide, aluminum oxide, platinum oxide, zirconium oxide, silicon oxide, tungsten oxide, silver oxide, yttrium oxide, colloidal gold, an ionic silver salt, elemental silver, titanium dioxide, bismuth pyrithione, zinc pyrithione, zinc percarbonates, zinc perborates, bismuth salts, arsenic trioxide, arsenicals, or combinations thereof.

In some embodiments, the microparticle comprises colloidal silver, metallic silver, silver chloride, silver bromide, silver phosphate, silver nitrate, silver citrate, silver acetate, silver benzoate, silver pyrithione, or combinations thereof. Silver microparticles having a particle size of about 1 to about 100 um or 5 and 200 um are generally understood to slowly release antimicrobial silver ions (e.g., Ag⁺).

In some embodiments, the release rate of metal ions (e.g., silver ions) depends on the initial concentration and size of the microscale particles. In certain embodiments, the release rate of metal ions (e.g., silver ions) is an indicator of the biocidal activity of the microscale particles. For example, in some instances, silver microscale particles in an aqueous environment oxidize in the presence of oxygen and protons according to the stoichiometric reaction:

$${Ag}_{( s )} + \frac{1}{2} {O}_{2} + 2 {H}_{( aq )}^{+} ↔ 2 {Ag}_{( aq )}^{+} + {H}_{2} {O}_{( l )}$$

releasing Ag+ ions during particle dissolution.

In further or alternative embodiments, the microparticle comprises titanium dioxide in the anatase phase. In some embodiments, the microscale titanium dioxide is photocatalytically active. Titanium dioxide in the anatase phase is generally understood to promote oxidation-reduction (redox) reactions when irradiated with ultraviolet or visible light. In some embodiments, a microparticle containing titanium dioxide that is irradiated with visible or ultraviolet light in an aqueous environment (e.g., within a microorganism) and in certain situations produce hydroxyl ions (OH⁻), superoxide ions (O₂⁻), and/or hydrogen peroxide (H₂O₂). In additional or further embodiments, a microscale particle containing titanium dioxide that is exposed to visible light while in a cell or in contact with a cell produces a toxic environment and damages or kills the cell. In some embodiments, the titanium dioxide oxidizes organic material (e.g. microbes).

In certain embodiments, suitable copper microscale particles include cupric oxide, cuprous oxide, cuprous iodide, cupric iodide, cupric phosphate, copper (II) hydrogen phosphate, and cupric silicate.

In some embodiments, the microscale particles have an average particle size between 0.1 and 500 um, 0.1 and 400 um, 0.1 and 300 um, 0.1 and 250 um, 0.1 and 200 um, 0.1 and 100 um, 0.1 and 90 um, 1 and 500 um, 1 and 450 um, 1 and 400 um, 1 and 350 um, 1 and 300 um, 1 and 250 um, 1 and 225 um, 1 and 200 um, 1 and 175 um, 1 and 150 um, 1 and 125 um, 1 and 100 um, 1 and 75 um, 1 and 50 um, 1 and 40 um, 1 and 30 um, 1 and 25 um, 1 and 20 um, 1 and 15 um, or 1 and 10 um. In some embodiments, the microscale particles have an average particle size between 5 and 10 um, 5 and 30 um 10 and 250 um, 10 and 200 um, 50 and 200 um, or 100 and 200 um. In further or additional embodiments, the microscale particles have an average particle size of less than about 500 um, less than about 450 um, less than about 400 um, less than about 350 um, less than about 300 um, less than about 250 um, less than about 200 um, less than about 175 um, less than about 150 um, less than about 125 um, less than about 100 um, less than about 95 um, less than about 90 um, less than about 85 um, less than about 80 um, less than about 75 um, less than about 70 um, less than about 65 um, less than about 60 um, less than about 55 um, less than about 50 um, less than about 45 um, less than about 40 um, less than about 35 um, less than about 30 um, less than about 28 um, less than about 25 um, less than about 23 um, less than about 20 um, less than about 18 um, less than about 15 um, or less than about 10 um. In further or additional embodiments, the microscale particles have an average particle size of about 500 um, about 400 um, about 350 um, about 300 um, about 250 um, about 200 um, about 175 um, about 150 um, about 125 um, about 100 um, about 75 um, about 60 um, about 50 um, about 25 um, or about 10 um. In some embodiments, the formulation comprises a bimodal distribution of particle sizes. In some embodiments, the formulation comprises a polydisperse population of particle sizes.

In some instances, for cases with similar silver concentrations by mass, samples with smaller particle size have a larger number concentration. In some instances, the increase in concentration of silver microcrystals leads to a higher microparticle:bacterium ratio and a greater amount of available microparticle surface area.

In some embodiments, the rate of release of Ag+ ions is dependent upon the size of the microcrystal, with smaller particles dissolving more readily. In some instances, the rate of release of microscale ions (e.g., silver microscale ions) is inversely proportional to the size of the microscale particle (e.g., microscale colloidal silver).

Some embodiments provided herein describe microscale particles having a diversity of shapes. In some embodiments, the microscale particles are spherical in shape. In other embodiments, the microscale particles are round plates, triangular plates, square plates, or hexagonal plates. In certain embodiments, the microscale particles are triangular plates. In some embodiments, the microscale particles are microrods, hexagonal-shaped, cube-shaped, polyhedron-shaped, or star-shaped. In some embodiments, the formulation comprises a bimodal distribution of particle shapes. In some embodiments, the formulation comprises a polydisperse population of particle shapes.

In some embodiments, the microscale particles comprise composites. Non limiting examples of suitable microscale particles include alloy of silver containing about 2.5 wt % copper, alumina-silver microscale composite, titania-silver microscale composite, silver-copper microscale composite, silver-iron oxide microscale composite, silver-silica microscale composite, and silver-selenium microscale composite. In some embodiments, the formulation comprises a microparticle/polymer composite film (e.g., silver microparticle/polyvinylpyrrolidone (PVP), silver microparticle/polyvinyl alcohol (PVA), etc.)

Some embodiments provided herein describe one or more microscale particles optionally comprising a capping ligand or stabilizing agent. Non-limiting examples of capping or stabilizing agents include polyvinylpyrrolidone (PVP), polyethyleneimine, citrate, keratin, tannic acid, bovine serum albumin (BSA), ionic surfactants (e.g, sodium dodecyl sulfate (SDS)), non-ionic surfactants (e.g., Tween 80), linoleic acid, poly(methylvinylether-co-maleic anhydride (PVM/MA), and sophorolipid.

In some embodiments, the microscale particle has antimicrobial activity. In some embodiments, the microscale particle is attached to a spore. In further or additional embodiments, the microscale particle is an active agent. In yet a further embodiment, the microscale particle kills the microbe after germination. In still further or additional embodiments, the microscale particle is an odor control agent, antimicrobial surface coating, self-cleaning surface coating, germicide, antibacterial agent, anti-microbial, anti-fungal, anti-viral agent, anti-protozoal agent, microbiostat, or disinfectant.

### Film-Forming Polymer

In some embodiments, the formulation or composition comprises a film-forming polymer. In some instances, the film-forming polymer leaves a protective film on the surface of the skin either immediately or upon evaporation of volatiles in the composition. In further embodiments, the film-forming polymer improves the water-, sweat-, transfer- and wear-resistance properties of the formulation or composition. In certain aspects, the film-forming polymer enhances the spread characteristics of the composition, which allows the composition to be more uniformly and consistently applied to skin or an article surface. In some embodiments, the film-forming polymer improves smoothness of the formulation. In some instances, the film-forming polymer is a penetration enhancer.

In further or additional embodiments, when used with one or more active agent(s), the film-forming polymer maintains the active agent at the surface of the skin or article for a longer period of time than it would otherwise remain without the film-forming polymer. In some embodiments, the film-forming polymer affords controlled release of the one or more active agent(s). In further or additional embodiments, the film-forming polymer affords sustained release of the one or more active agent(s). In further or alternative embodiments, the film-forming polymer affords immediate release of the one or more active agent(s). In further or alternative embodiments, the film-forming polymer affords immediate and controlled release of the one or more active agent(s). In yet further or additional embodiments, the film-forming polymer affords sustained release of the one or more active agent(s).

In certain embodiments, the film-forming polymer (e.g., polyprepolymer) suspends the antimicrobial (e.g., nanoparticle or microparticle) to form a long lasting liquid reservoir in the stratum corneum and epidermis. In further or additional embodiments, the film-forming polymer (e.g., polyprepolymer) significantly influences the deposition of the antimicrobial (e.g., nanoparticle or microparticle) on the surface of the skin or hard surface. In certain embodiments, the antimicrobial (e.g., nanoparticle or microparticle) and film-forming polymer (e.g., polyprepolymer) remain on the surface of the skin or hard surface and does not penetrate. In some embodiments, the antimicrobial (e.g., nanoparticle or microparticle) is suspended and remains active on the surface of the skin where it is most efficacious. In some embodiments, the film-forming polymer (e.g., polyprepolymer) provides an increased bioavailability and/or safety profile with respect to the antimicrobial formulation. In some embodiments, the film-forming polymer (e.g., polyprepolymer) prevents agglomeration of the antimicrobial (e.g., nanoparticle or microparticle). In some embodiments, the film-forming polymer prevents protein binding (e.g., non-specific protein binding) of the microbial (e.g., nanoparticle or microparticle). The formulations and compositions described herein provide a more safe and efficacious advancement in the art compared to existing technologies.

In some embodiments, the film-forming polymer is a synthetic polymer, a polymer of natural origin or mixture thereof.

Examples of film-forming polymers include, but are not limited to, one or more acrylate copolymers such as acrylate/octylacrylamide copolymers and acrylate/vinyl acetate copolymers; cellulosic polymers such as methyl cellulose and hydroxyethyl cellulose; ethylene/acrylic acid copolymer; polyacrylic acid; C₁ to C₅ alkyl galactomannan; isododecane/ethylene mixed copolymer; adipic acid/diethylene glycol/glycerin crosspolymer; trimethylpentanediol/adipic acid copolymer; trimethylpentanediol/adipic acid/isononanoic acid; polyvinyl pyrrolidone copolymer, PVP/hexadecene copolymer (e.g., Ganex V-216); PVP/eicosene copolymer (e.g., Ganex V-220); PVP/tricontanyl copolymer (e.g., Ganex WP-660); PVP/vinyl acetate copolymer; allyl stearate/vinyl acetate copolymers; alpha olefin/isopropyl maleate/MA polymer; cycloalkyl methacrylate copolymer/isododecane trimethyl polysiloxane; octadecene/MA copolymer; polypropylene glycol/sodium maleic acid diisobutylene copolymers (e.g., PPG-12/SMDI copolymer and PPG-51/SMDI, available from Penederm Inc.); poly(styrene-co-maleic anhydride) copolymers (SMA); acrylates C₁₀ to C₃₀ alkyl acrylate crosspolymer; cetyl hydroxyethylcellulose; dimethiconol; dimethicone; diglycol/cyclohexane-dimethanol/isophthalates/sulfoisophthalate copolymer; polyethylene; ethoxydiglycol; waxes such as beeswax and botanical waxes; polyurethane, polyurethane resins; natural gums; or any combinations of these ingredients. The polyurethane resins include Polyurethane-1, Polyurethane-2, Polyurethane-4, Polyurethane-5, and mixtures thereof. Additional film formers include those set forth in U.S. Pat. No. 5,916,541, which is incorporated herein by reference.

In some embodiments, the film-forming polymer comprises polyolprepolymer-2 (PPG-12/SMDI), polyolprepolymer-14 (PPG-51/SMDI), poly(styrene-co-maleic anhydride) copolymers (SMA); acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In some embodiments, any suitable film-forming polymer is used.

In certain embodiments, the film-forming polymer is an oil soluble penetration enhancer. In other embodiments, the film-forming polymer is a water soluble penetration enhancer.

### Additional Active Agents

In some embodiments, the present composition optionally includes one or more of the following additional ingredients: anesthetics, anti-allergenics, antifungals, antimicrobials, anti-inflammatories, antiseptics, chelating agents, colorants, depigmenting agents, emollients, exfollients, fragrances, humectants, lubricants, moisturizers, pharmaceutical agents, preservatives, skin protectants, skin penetration enhancers, stabilizers, surfactants, thickeners, viscosity modifiers, and vitamins.

In some embodiments, any formulation or composition described herein further comprises a source of iodine. Non-limiting examples of a source of iodine include iodine, iodophors, tincture of iodine, iodine salts, povidone-iodine, and combinations thereof. In some embodiments, any formulation or composition described herein further comprises any suitable iodophor. In some embodiments, any formulation or composition described herein further comprises a source of betadiene. In some specific embodiments, any formulation or composition described herein further comprises ethanol. In some other embodiments, any formulation or composition described herein further comprises isopropanol. In further embodiments, any formulation or composition described herein further comprises chlorohexidene.

In some embodiments, any formulation or composition described herein further comprises a surfactant. Examples of surfactants include but are not limited to quaternary ammonium salt benzalkonium ions (e.g., benzalkonium chloride), poly(ethyleneimine), DAXAD 19 (distributed by GEO Specialty Chemicals), benzethionium chloride, cetyl trimethyl ammonium chloride, trimethyl coco quaternary ammonium chloride, diquaternary polydimethylsiloxane, tris(2-hydroxyethylamine) benzyl ammonium chloride, monoalkyltrimethylammonium salts, dialkyldimethylammonium salts, heteroaromatic ammonium salts, polysubstituted quaternary ammonium salts, bis-quaternary ammonium salts, and polymeric quaternary ammonium salts, cocamidopropyldimethyl betaine, and trimethylquaternary ammonium chloride. In some embodiments, the surfactant is n-alkyl dimethylbenzylalkonium chloride, wherein said n-alkyl group is 10 to 20, 10 to 18, 10 to 16, 12 to 16, or 10 to 14 carbons in length. In some embodiments, the surfactant is Stepanquat® 50 NF. In certain embodiments, the surfactant has antimicrobial properties. In some embodiments, the formulation or composition further comprises any quaternary amines suitable for a bactericide. In some embodiments, the surfactant is a benzalkonium homolog having the structure of:

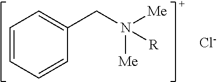

wherein R is an alkyl chain ranging from 10 to 17, 10 to 15, 10 to 18, 10 to 16, 12 to 16, or 10 to 14 carbon atoms. Non-limiting examples of these homologs include N,N-dimethyldecylammonium chloride, N,N-dimethylundecylammonium chloride, N,N-dimethyldodecylammonium chloride, N,N-dimethyltridecylammonium chloride, N,N-dimethyltetradecylammonium chloride, N,N-dimethylpentadecylammonium chloride, N,N-dimethylhexadecylammonium chloride, N,N-heptadecylammonium chloride, and combinations thereof.

In certain embodiments, any composition or formulation described herein further comprises a coloring agent, photochromic agent or photoactive agent. In certain embodiments, a coloring agent or photochromic pigment provides as a visual signal or indicator of antimicrobial protection. In some embodiments, a color change or color fade indicates to the user that re-application is necessary to maintain antimicrobial protection. In further or additional embodiments, the coloring agent provides a color change or fade after 0.5 h, 1 h, 2 h, 3 h, 6 h, 12 h, 15 h, 18 h, 24 h, 36 h, 48 h, 3 days, 4 days, 5 days, 6 days, 1 week, 2 weeks, 3 weeks, 1 month, 2 months, 3 months, 4 months, 5, months, 6 months, 7 months, 8 months, 9 months, 10 months, 11 months, 1 year, or 2 years. In some embodiments, the present composition is clear or colorless when applied, and becomes colored when re-application is necessary to maintain antimicrobial protection. In further or alternative embodiments, the present composition is colored when applied, then becomes colorless, clear or faded when re-application is necessary to maintain antimicrobial protection. In certain embodiments, the present composition is clear or colorless in outdoor light, and becomes colored in indoor light. For example, when used in medical and/or pharmaceutical applications, the compositions described herein are topically applied as an anti-microbial during outpatient surgery to indicate duration/wear of the anti-microbial. The applied composition remains colored during the length of the surgery to indicate that, for example, the anti-microbial is still taking effect. As the color intensity begins to fade, the surgeon knows that the effect of the anti-microbial is beginning to wear off. However, even if the surgery is completed before the anti-microbial composition or formulation wears off, the composition becomes clear or colorless when the person exits the outpatient facility and is exposed to outdoor light. In some instances, the coloring agent or photochromic material is colorless at a light intensity greater that about 1 Joule/cm², preferably greater than about 2 Joules/cm², and most preferably greater than about 5 Joules/cm². In other instances, the coloring agent or photochromic material is colored at a light intensity less than about 5 Joules/cm², preferably less that about 2 Joules/cm² and most preferably less that about 1 Joule/cm².

In some embodiments, organic photochromic compounds that are used in the present compositions and formulations include, but are not limited to azobenzene compounds, thioindigo compounds, dithizone metal complexes, spiropyran compounds, spirooxazine compounds, napthopyran compounds, fulgide compounds, dihydropyrene compounds, spirothiopyran compounds, 1,4-2H-oxazine, triphenylmethane compounds, viologen compounds, or any combinations thereof. In specific embodiments, organic photochromic compounds that are used in the present compositions and formulations include, but are not limited to, 1,3,3-trimethylspiro[indolino-2,3′(3H)naphtho(2,1-b)(1,4,)-oxazine]; 5-methoxy-1,3,3-trimethylspiro[indolino-2,3′-(3H)naptho(2,1-b)(1,4)-oxazine]; 5-chloro-1,3,3-trimethylspiro[indolino-2,3′-(3H)naphtho(2,1-b)(1,4)-oxazine]; 8′-piperidino-1,3,3-trimethylspiro[indolino-2,3′-(3H)naphtho(2,1-b)(1,4)-oxazine]; 1-benzyl-3,3-dimethyspiro[indolino-2,3′-d(3H)naphtho(2,1-b)(1,4)-oxazine]; 1,3,5,6,-tetramethyl-3-ethylspiro[indolino-2,3′-(3H)naphtho(2,1-b)(1,4)-oxazine]; 1,3,3,5,6-pentamethylspiro[indolino-2,3′-(3H)naphtho(2,1-b)(1,4)oxazine]; 1,3′,3′-trimethylspiro(2H-1benzopyran-2,2′-indolino); 3,3,1-diphenyl-3H-naphtho-(2,1,1-b)pyran; 1,3,3-triphenylspiro[indolino-2,3′-(3H)naphtho(2,1-b)pyran]; 1-(2,3,4,5,6-pentamethylbenzyl)-3,3-dimethylspiro[indolino-2,3′-(3H)-naphtho(2,1-b)pyran]; 1-(2-nitrobenzyl)-3,3-dimethylspiro[indolino-2,3′-(3H)-naphtho(2,1-b)pyran]; 1,1-diphenylnaphthopyran, 2,5-dimethylfuryl-trimethyfulgide, 2-methyl-5-chlorotrimethylfulgide, or any combinations thereof. In certain embodiments, photochromic and thermochromic material are used in combination.

In certain embodiments, any composition or formulation described herein further comprises one or more moisturizing agents. In specific embodiments, moisturizing agents that are used in the present compositions and formulations include, but are not limited to various polyethylene glycols (e.g., PEG 4, PEG 6, PEG 8, PEG 12 and PEG 20), sorbitol, propylene glycol monostearate, glycerin, fatty acid esters of α-tocopherol (e.g., linoleic acid ester of α-tocopherol, oleic acid ester of α-tocopherol, linolenic acid ester of α-tocopherol, palmitic acid of α-tocopherol, stearic acid ester of α-tocopherol, and myristic acid ester of α-tocopherol), oils (e.g., mineral oil, carob bean oil, palm oil, cabbage palm oil, coconut oil, jojoba oil, sunflower seed oil, high oleic sunflower oil, grapeseed oil, black mustard oil, ocilet oil, shea butter, sweet almond oil, soya-bean oil, avocado oil, peanut oil, cottonseed oil, sesame oil, olive oil, maize oil, coconut butter, castor oil, Ben oil, linseed oil, colza oil, annato oil, cornseed oil, safflower oil, walnut oil, hazelnut oil rapeseed oil, horsehair oil, mink oil, turtle oil, whale oil, fish oil, fish-liver oil, soft-roe oil, neat's-foot oil, tallows and egg oil), aloe extracts, mucopolysaccharides, collagen, lecithin, squalene, panthenol (e.g., D-panthenol), Hydromide® Blend, Liponate® GC, vitamin D3, ceramide, pseudoceramide, phytosterols (e.g., Net Sterol 100), hyaluronic acid, sodium hyaluronate, xylitolglucoside, xylitol, anhydroxylitol, sodium pyrollidone carboxylate, sodium lactate, orotic acid, propylene glycol, honey, petrolatum, lanolin, silicones (e.g., dimethicone), fatty acids, fatty acid esters, cholesterol, keratin and elastin.

In yet other embodiments, the composition comprises at least one sunscreen, sunprotectant or sunblock agent. “Sunscreen”, “sunprotectant” or “sunblock” as used herein defines ultraviolet ray-blocking compounds exhibiting absorption or blockage within the wavelength region between about 290 and 420 nm. Such agents are classified into five groups based upon their chemical structure: para-amino benzoates; salicylates; cinnamates; benzophenones; and miscellaneous chemicals including menthyl anthralinate and digalloyl trioleate. Inorganic sunscreens that are optionally used include titanium dioxide, zinc oxide, iron oxide and polymer particles such as those of polyethylene and polyamides. Specific suitable sunscreen agents include, for example: p-aminobenzoic acid, its salts and its derivatives (ethyl, isobutyl, glyceryl esters; p-dimethylaminobenzoic acid); Anthranilates (i.e., o-aminobenzoates; methyl, menthyl, phenyl, benzyl, phenylethyl, linalyl, terpinyl, and cyclohexenyl esters); Salicylates (amyl, phenyl, benzyl, menthyl, glyceryl, and dipropylene glycol esters); Cinnamic acid derivatives (methyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,3-disulfonic and of 2-naphthol-6,8-disulfonic acids); Dihydroxynaphthoic acid and its salts; o- and p-Hydroxybiphenyidisulfonates; Coumarin derivatives (7-hydroxy, 7-methyl, 3-phenlyll); Diazoles (2-acetyl-3-bromoindazole, phenyl benzoxazole, methyl naphthoxalole, various aryl benzothiazoles); Quinine salts (bisulfate, sulfate, chloride, oleate, and tannate); quinoline derivatives (8-hydroxyquinoline salts, 2-phenylquinoline); Hydroxy- or methoxy substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzene, Sulisobenzone, Dioxybenzone, Benzoresorcinol, 2,2′,4,4′-Tetrahydroxybenzophenone, 2,2′-Dihydroxy4,4′-dimethoxybenzophenone, Octabenzone; 4-Isopropyhldibenzoylmethane; Butylmethoxydibenzoylmethane; Etocrylene; and 4-isopropyl-di-benzoylmethane; titanium dioxide, iron oxide, zinc oxide, and mixtures thereof. Other cosmetically-acceptable sunscreens and concentrations (percent by weight of the total cosmetic sunscreen composition) include diethanolamine methoxycinnamate (10% or less), ethyl-[bis(hydroxypropyl)]aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less). Yet other cosmetically-acceptable sunscreens and concentrations (reported as a percentage by weight of the total cosmetic sunscreen composition, and referring to the final percentage of the sunscreen) include: aminobenzoic acid (also called para-aminobenzoic acid and PABA; 15% or less; a UVB absorbing organic sunscreen), avobenzone (also called butyl methoxy dibenzoylmethane; 3% or less, a UVA I absorbing organic sunscreen), cinoxate (also called 2-ethoxyethyl p-methoxycinnamate; 3% or less, a UVB absorbing organic sunscreen), dioxybenzone (also called benzophenone-8; 3% or less, a UVB and UVA II absorbing organic sunscreen), homosalate (15% or less, a UVB absorbing organic sunscreen), menthyl anthranilate (also called menthyl 2-aminobenzoate; 5% or less, a UVA II absorbing organic sunscreen), octocrylene (also called 2-ethylhexyl-2-cyano-3,3 diphenylacrylate; 10% or less, a UVB absorbing organic sunscreen), octyl methoxycinnamate (7.5% or less, a UVB absorbing organic sunscreen), octyl salicylate (also called 2-ethylhexyl salicylate; 5% or less, a UVB absorbing organic sunscreen), oxybenzone (also called benzophenone-3; 6% or less, a UVB and UVA II absorbing organic sunscreen), padimate O (also called octyl dimethyl PABA; 8% or less, a UVB absorbing organic sunscreen), phenylbenzimidazole sulfonic acid (water soluble; 4% or less, a UVB absorbing organic sunscreen), sulisobenzone (also called benzophenone-4; 10% or less, a UVB and UVA II absorbing organic sunscreen), titanium dioxide (25% or less, an inorganic physical blocker of UVA and UVB), trolamine salicylate (also called triethanolamine salicylate; 12% or less, a UVB absorbing organic sunscreen), and zinc oxide (25% or less, an inorganic physical blocker of UVA and UVB).

In certain embodiments, any composition or formulation described herein further comprises any suitable chelating agents, which include but are not limited to EDTA (acid form), citric acid, hydroxyethylidene phosphonic acid, polyvinylphosphoric acid, polyvinylsulfonate, acrylic acid, phytic acid, sodium phytate, and aminophosphonic acid.

In certain embodiments, any composition or formulation described herein further comprises one or more essential oil. In specific embodiments, essential oils that are optionally used in the present compositions and formulations include, but are not limited to cinnamon oil, clove oil, eucalyptus oil, garlic oil, oregano oil, jojoba oil, lavender oil, leleshwa oil, lemon oil, lemon tea tree oil, lemon myrtle oil, mint oil, neem oil, nigella sativa oil, onion oil, peppermint oil, sandalwood oil, sideritis or greek mountain tea oil, tea tree oil, thyme oil, lemongrass oil, cedarwood oil, sage oil, vetiver oil, bay oil and any combinations thereof. In some embodiments, the essential oil has antimicrobial activity.

### Methods

Provided herein in some embodiments is a method of reducing the population of pathogenic microorganisms or killing at least one pathogenic microorganism. Also provided herein in some embodiments is a method of reducing the population of vegetative bacteria or partially germinated bacteria. Also provided herein is a method for killing at least one vegetative or partially germinated bacteria. In some embodiments, the compositions and formulations described herein are applied to skin surface. In some embodiments, the compositions and formulations described herein is not absorbed through the skin. In certain embodiments, the skin surface comprising the compositions described herein is further covered with a physical barrier (e.g., dressing, medical covering for a wound, bandage, gauzes, cloth, gloves, and plastic barrier coverings). In certain embodiments, the skin surface is substantially free of microorganisms for at least 0.5 h, 1 h, 2 h, 3 h, 4 h, 5 h, 6 h, 12 h, 24 h, 36 h, 48 h, 2 days, 3 days, 4 days, 5 days, 6 days, 1 week, 2 weeks, 3 weeks, or 1 month. In other embodiments, the skin surface is substantially free of microorganisms from about 1 h to about 1 month, about 1 h to about 1 week, about 1 h to about 3 days, about 1 h to about 2 days, about 1 h to about 1 day, about 1 h to about 12 h, about 1 h to about 6 h, about 1 h to about 3 h, 6 h to about 1 month, about 6 h to about 1 week, about 6 h to about 3 days, about 6 h to about 2 days, about 6 h to about 1 day, about 6 h to about 12 h, about 12 h to about 1 month, about 12 h to about 1 week, about 12 h to about 3 days, about 12 h to about 2 days, about 12 h to about 1 day, about 24 h to about 1 month, about 24 h to about 1 week, about 24 h to about 3 days, about 24 h to about 2 days, or about 24 h to about 36 h. In certain embodiments, the skin surface is substantially free of microorganisms from about 12 h to about 24 h. In certain embodiments, the skin surface is substantially free of microorganisms from about 12 h to about 24 h when the skin surface is further covered with a physical barrier.

Provided herein in other embodiments is a method of reducing the population of pathogenic microorganisms or killing at least one pathogenic microorganism on an article surface. In some instances, the surface is hard, soft, smooth, non-porous, or porous. Examples of article surfaces include but are not limited to glass, ceramic, metal, plastic, paper, silicate, polymer or polymer/wood composites. In some embodiments, the surface is porous. In further or alternative embodiments, the surface is non-porous. Examples of porous surfaces include but are not limited to a mat of fibers, a zeolite, or a porous film. In certain embodiments, the antimicrobial composition slowly leaches from the formulation, keeping the coated surface free of live bacteria, yeasts, and molds. In further or additional embodiments, application of the composition to a surface, followed by exposure of the surface to visible or ultra-violet light, causes the destruction or inactivation of microbes or viruses that are present on the surface.

For example, in certain embodiments, a composition is present on a surface that is exposed to microbes, such as bacteria and fungi, and/or to viruses. Such a surface is a “disinfecting surface” by destroying or inactivating microbes or viruses that are present on the surface. For example, surfaces in residential, commercial or hospital environments may have a coating of an antimicrobial composition on the surface. Non-limiting examples of surfaces that may be made into disinfecting surfaces include countertops, flooring, walls, handles, telephones, and surfaces of medical instruments or devices.

In some embodiments provides the method comprising applying to the surface any one of the compositions described herein. In some other embodiments provides a method of accelerating germination of one or more bacterial endospores on a surface, wherein the method comprises applying to the surface a composition described herein.

In certain embodiments, the surface is a physical barrier used to cover skin surface (e.g., dressing, medical covering for a wound, bandage, gauzes, cloth, gloves, and plastic barrier coverings). In other embodiments, the surface is a medical device. In further or additional embodiments, examples of suitable medical devices include but are not limited to catheters (e.g., IV, Foley), heart valves, pacemakers, stents, gastrostomy tubes, feeding tubes, silicone coated latex type surfaces, silicone valves, balloons, septa, silicone parts used in various medical pumps, tubings, and earplugs, and as a textile finish for linings for hospital beds, window shades, and curtains.

In certain embodiments, the article surface is substantially free of microorganisms for at least 0.5 h, 1 h, 2 h, 3 h, 4 h, 5 h, 6 h, 12 h, 24 h, 36 h, 48 h, 2 days, 3 days, 4 days, 5 days, 6 days, 1 week, 2 weeks, 3 weeks, 1 month, 3 months, 6 months, 9 months, 1 year, 1.5 years, 2 years, or 5 years. In other embodiments, the article surface is substantially free of microorganisms from about 1 h to about 1 month, about 1 h to about 1 week, about 1 h to about 3 days, about 1 h to about 2 days, about 1 h to about 1 day, about 1 h to about 12 h, about 1 h to about 6 h, about 1 h to about 3 h, 6 h to about 1 month, about 6 h to about 1 week, about 6 h to about 3 days, about 6 h to about 2 days, about 6 h to about 1 day, about 6 h to about 12 h, about 12 h to about 1 month, about 12 h to about 1 week, about 12 h to about 3 days, about 12 h to about 2 days, about 12 h to about 1 day, about 24 h to about 1 month, about 24 h to about 1 week, about 24 h to about 3 days, about 24 h to about 2 days, about 24 h to about 36 h, about 1 week to about 5 years, about 1 week to about 2 years, about 1 week to about 1 year, about 1 week to about 6 months, about 1 week to about 3 months, about 1 week to about 1 month, about 1 month to about 2 years, about 1 month to about 1 year, about 1 month to about 9 months, about 1 month to about 6 months, about 1 month to about 3 months, or 1 month to about 2 months.

In some embodiments, the pathogenic microorganism is selected from, by way of non-limiting example, fungi, bacteria, viruses, protozoa, Gram-positive bacteria (e.g., Staphylococcus species, Streptococcus species, Bacillus species, and Clostridium species), Gram-negative bacteria (e.g., Escherichia species, Salmonella species, Aeromonas species, Klebsiella species and Campylobacter species), and combinations thereof. In specific embodiments, the pathogenic microorganism is selected from, by way of non-limiting example, Aspergillus niger, Pseudomonas aeruginosa, Staphylococcus aureus (MRSA), Clostridium difficile, carbapenem resistant Klebsiella pneumoniae and vancomycin-resistant Enterococci, influenza virus, H1N1 influenza virus, hepatitis A virus, hepatitis B virus, hepatitis C virus, HIV, rubella virus, human respiratory syncytial virus, mumps virus, Epstein-Barr virus, varicella zoster virus, measles virus (morbillivirus), and combinations thereof.

In some embodiments, the pathogenic microorganism is selected from the group that comprises Aeromonas hydrophila, Aeromonas sobria, Aeromonas caviae, Actinomyces israelii, Actinomyces naeslundii, Bacillus anthracis, Bacillus cereus, Bacillus subtilis, Bacillus thuringiensis, Bacillus stearothermophilus, Bacteroides fragilis, Bordetella pertussis, Bordetella parapertussis, Bordetella bronchiseptica, Borrelia recurrentis, Borrelia burgdorferi, Brucella abortus, Brucella canis, Brucella melintensis, Brucella suis, Burkholderia pseudomallei, Burkholderia cepacia, Campylobacter jejuni, Campylobacter coli, Campylobacter lari, Campylobacter fetus, Clostridium perfringens, Clostridium difficile, Clostridium botulinum, Corynebacterium diphtheria, Corynebacterium jeikeum, Corynebacterium urealyticum, Edwardsiella tarda, Citrobacter freundii Citrobacter diversus, Enterobacter aerogenes, Enterobacter agglomerans, Enterobacter cloacae, Escherichia coli, Klebsiella pneumoniae, Klebsiella oxytoca, Morganella morganii, Proteus mirabilis, Proteus vulgaris, Providencia alcalifaciens, Providencia rettgeri, Providencia stuartii, Salmonella enteric, Salmonella typhi, Salmonella paratyphi, Salmonella enteritidis, Salmonella cholerasuis, Salmonella typhimurium, Serratia marcesans, Serratia liquifaciens, Shigella dysenteriae, Shigella flexneri, Shigella boydii, Shigella sonnei, Yersinia enterocolitica, Yersinia pestis, Yersinia pseudotuberculosis, Enterococcus faecalis, Enterococcus faecium, Erysipelothrix rhusopathiae, Francisella tularensis, Haemophilus influenzae, Haemophilus ducreyi, Haemophilus aegyptius, Haemophilus parainfluenzae, Haemophilus haemolyticus, Haemophilus parahaemolyticus, Helicobacter pylori, Helicobacter cinaedi, Helicobacter fennelliae, Klebsiella pneumoniae, Legionella pneumophila, Listeria monocytogenes, Mycobacterium leprae, Mycobacterium tuberculosis, Nocardia asteroids, Nocardia brasiliensis, Neisseria gonorrhoeae, Neisseria meningitides, Pasteurella multocida, Proteus vulgaris, Proteus mirabilis, Salmonella enteric, Salmonella typhi, Salmonella paratyphi, Salmonella enteritidis, Salmonella cholerasuis, Salmonella typhimurium, Shigella dysenteriae, Shigella flexneri, Shigella boydii, Shigella sonnei, Staphylococcus aureus, Staphylococcus epidermidis, Staphylococcus saprophyticus, Streptococcus pyogenes, Streptococcus agalactiae, Streptococcus anginosus, Streptococcus equismilis, Streptococcus bovis, Streptococcus mutans, Streptococcus salivarius, Streptococcus sanguis, Streptococcus mitis, Streptococcus milleri, Treponema pallidum, Treponema pallidum, Treponema pallidum, Treponema carateum, Vibrio cholerae, Vibrio parahaemolyticus, Vibrio vulnificus, Vibrio alginolyticus, Vibrio mimicus, Vibrio hollisae, Vibrio fluvialis, Vibrio metchnikovii, Vibrio damsel, Vibrio furnisii, Yersinia enterocolitica, Yersinia pestis, Yersinia pseudotuberculosis, Rhinovirus, Influenzavirus, Herpes simplex virus HIV, Ebolavirus, Saccharomyces cerevisiae, Pityrosporum ovale, Malassezia furfur, Candida albicans, Cryptococcus neoformans, Aspergillus, Rhizopus, Mucor, or any combination thereof.

In some certain embodiments, the bacterial endospores comprise Bacillus or Clostridium species, or combinations thereof.

In further embodiments, the endospore-forming bacteria comprise Clostridium difficile, Clostridium perfringens, Clostridium sporogenes, Bacillus subtilis, Bacillus circulans, Bacillus pumilus, Bacillus cereus, Bacillus stearothermophilu, Bacillus anthracis, Bacillus globigii, or combinations thereof.

Provided herein, in some embodiments, is a formulation comprising nanoscale particles (e.g., nanoscale colloidal silver), wherein the release of ions (e.g., silver ions) followed by increased membrane permeability, loss of the proton motive force, inducing de-energization of the cells and efflux of phosphate, leakage of cellular content, disruption of DNA replication or combinations thereof leads to anti-bacterial or anti-microbial activity.

In some instances, the uptake of metal ions and the interactions with DNA and proteins within the bacteria (e.g., Gram-positive and Gram-negative bacteria) provides anti-bacterial or biocidal activity. In some instances, the metal ions bind with phosphate groups on DNA chains to block transcription or causes detrimental mutations. In other instances, the metal ions optionally bind to thiol groups on proteins that regulate respiration within the cell and interfere with these processes, leading to cell death. In some instances, silver and other heavy metal ions optionally catalyze the production of reactive oxygen species beyond concentrations that the cells can control, leading to attacks on cell membranes and DNA damage.

In some instances, direct interactions between metal nanoscale or microscale particles and the cell wall of a Gram-negative bacterium leads to anchoring of the particle onto the cell wall or uptake of the particle into the interior of the cell. In some embodiments, these interactions lead to cell death. In some instances, shape-dependent interactions affect biocidal activity. In some instances, the shape of the nanoscale particle increases the disruptive effects of the nanoscale particles binding to bacteria cell wall.

### Formulation

Certain embodiments described herein provide the formulation as an oral care product, over-the-counter drug, over-the-counter pharmaceutical, suncare product, sunscreen product, foot-care product, liquid and bar soap, cleaning product, self-cleaning product, sanitizing product, antiperspirant product, deodorant product, fragrance product, insect repellant, cosmetic product, hair care product, shampoo, hair conditioner, hair spray, moisturizers or combinations thereof. In some embodiments, the compositions are in the form of tablets, capsules, skin patches, inhalers, eye drops, nose drops, ear drops, suppositories, creams, ointments, injectables. In certain embodiments, the product form of the present compositions are in the form of an aerosol, cream, foam, emulsion, gel, liquid, lotion, mousse, patch, pomade, powder, solid, spray, stick or towelette, or any combinations thereof.

In certain embodiments, the formulations described herein provide immediate and sustained release of one or more active agent. In other embodiments, the formulations described herein provide sustained release of one or more active agent. In further or alternative embodiments, the formulations described herein provide immediate release of one or more active agent.

In some embodiments, the compositions described herein constitute protection, treatment or care creams, sanitizers, milks, lotions, gels or foams for the face, for the hands, for the body and/or for the mucous membranes, or for cleansing the skin, or for disinfecting surfaces, or for cleansing surfaces. In certain embodiments, the compositions consist of solid preparations constituting soaps or cleansing bars. In some embodiments, the emulsions cover a broad range of consistencies including a thin lotion (which, in some instances, is also suitable for spray or aerosol delivery), creamy lotion, light cream, and heavy cream. Other suitable topical carriers include an anhydrous liquid solvent such as oil and alcohol; aqueous-based single phase liquid solvent (e.g. hydro-alcoholic solvent system); anhydrous solid and semisolid (such as a gel and a stick); and aqueous based gel and mousse system.

In certain embodiments, the nanoscale particle(s) or the microscale particle(s), film-forming polymer, and optional active agents are administered in the form of a composition suitable for pharmaceutical, cosmetic and industrial applications. The compositions disclosed herein may contain a pharmacologically, cosmetically or industrially acceptable carrier. Such carriers are compatible with skin, nails, mucous membranes, tissues, hair, and/or surfaces. In some embodiments, the compositions disclosed herein are in any form suitable for topical application, including aqueous, aqueous-alcoholic or oily solutions, lotion or serum dispersions, aqueous, suspension, solution, mixture, homogeneous phase formulation, anhydrous or oily gels, emulsions obtained by dispersion of a fatty phase in an aqueous phase (O/W or oil in water) or, conversely, (W/O or water in oil), microemulsions or alternatively microcapsules, multiple phase emulsions, microparticles or lipid vesicle dispersions of ionic and/or nonionic type. In some embodiments, the compositions disclosed herein comprise alcohol (e.g., SD Alcohol SDA 40-2 190 Proof, cetyl alcohol, etc.). In some embodiments, the compositions disclosed herein are alcohol-free. In some embodiments, the compositions disclosed herein are formulated as composite films, paints or fibers.

In other embodiments, the composition further comprises at least one of water, a preservative, a surfactant (e.g., Incromine® Oxide C), an antioxidant (vitamin E acetate), an emulsifier (e.g. Emulium® Kappa), a conditioner, an emollient, a wax (e.g., Cutina® CP), an oil, a polymer, a pH adjuster (e.g., AMP Ultra® PC 2000) an adjuvant (e.g., hydrophilic or lipophilic gelling agents), a thickener (e.g., Cosmedia® Ultragel 300, Structure® Solanace, Keltrol® xanthan gum, etc.), a fixative, a colorant, a humectant, a moisturizer, a stabilizer, a diluent, a solvent (e.g, Dermofeel® TC-7), a filler, a sunscreen, an odor-absorber, a dyestuff, and a fragrance.

In some embodiments, the compositions and formulations described herein further comprise a vehicle acceptable for topical application to the skin or hair. Examples of such vehicles include, but are not limited to, water and aqueous systems (e.g., deionized water, sterile water); glycerin; various hydrophilic solvents including alcohols such as ethanol, methanol, propyl and other alcohols; polyglycols (e.g., glycerol or polyethylene glycol), esters of fatty acids, oils, fats, silicones, and the like.

In some embodiments, any composition or formulation described herein optionally comprises at least one preservative. Suitable preservatives include, but are not limited to, acids, alcohols, glycols, parabens, sorbates (e.g., potassium sorbate), quaternary-nitrogen containing compounds, isothiazolinones, aldehyde-releasing compounds and halogenated compounds. Illustrative alcohols include phenoxyethanol, isopropyl alcohol, and benzyl alcohol; illustrative glycols include propylene, butylene and pentylene glycols (e.g., 1,3-butylene glycol); illustrative parabens include (also known as parahydroxybenzioc acids) methyl, propyl and butyl parabens; illustrative quaternary nitrogen containing compounds include benzalkonium chloride, Quartenium 15; illustrative isothiazoles include methylisothiazoline, methychlorolisothiazoline; illustrative aldehyde releasing agents include DMDM hydantion, imiadolidinyl urea and diazolidinyl urea; illustrative antioxidants include butylated hydroxytoluene, tocopherol and illustrative halogenated compounds include triclosan and chlorohexidene digluconate. In some embodiments, any of the compositions described herein optionally comprise Euxyl® PE 9010. In some embodiments, any of the compositions described herein optionally comprise cocamidopropyl PC-dimonium chloride phosphate (e.g., Arlasilk® PTC). Examples of preservatives useful for the purpose of the present disclosure can be found in Steinberg, D. “Frequency of Use of Preservatives 2007”. Cosmet. Toilet. 117, 41-44 (2002) and, “Preservative Encyclopedia” Cosmet. Toilet. 117, 80-96 (2002). In addition, enzyme preservative systems such as those described in the article by Ciccognani D. Cosmetic Preservation Using Enzymes, in “Cosmetic and Drug Microbiology”, Orth DS ed., Francis & Taylor, Boca Raton, Fla. (2006) can also be effective for use with the composition of the present disclosure.

Compositions disclosed herein are formulated in conventional manner using one or more pharmaceutically, cosmetically, or industrially acceptable carriers comprising excipients and auxiliaries which facilitate processing of the nanoscale or microscale particle(s), film-forming polymer, and optional agents. Proper formulation is dependent upon the route of administration chosen and standard therapeutic practice. As used herein, the term “pharmaceutically, cosmetically or industrially acceptable carrier” means an inert, non toxic solid or liquid filler, diluent or encapsulating material, not reacting adversely with the active compound or with the subject. Suitable carriers are well known, and include water, saline, aqueous dextrose, sugar solutions, ethanol, glycols and oils, including those of petroleum, animal, vegetable, or synthetic origin, for example, peanut oil, soybean oil and mineral oil. In other embodiments, an active agent or combination of active agents described herein is optionally formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate, sodium lauryl sulfate and BRIJ® IC20-70), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Some embodiments provided herein describe a sprayable formulation comprising nanoscale particles (e.g., silver nanocrystals) or microscale particles (e.g., silver microcrystals). In some embodiments, the formulation comprises a bimodal distribution of particle sizes, wherein the smaller particles provide immediate and high ion release rates to kill microbes (e.g., bacteria) on surfaces and the larger particles provide a long-term ion source. In other embodiments, the formulation comprises a polydisperse size population of particles. In some embodiments, the formulation optionally comprises a biocompatible polymer. In some embodiments, one or more polymer additives provide a thin film to adhere the formulation to surfaces. In some embodiments, the polymer film is hygroscopic. In some instances, the hygroscopic film absorbs water from the atmosphere to enhance silver oxidation and/or provides channels for ion transport.

For oral administration, the compositions, in some embodiments, take the form of, for example, tablets or capsules prepared by conventional means with acceptable excipients or carriers such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolae); or wetting agents (e.g., sodium lauryl sulphate). Liquid preparations for oral administration are, in certain embodiments, solutions, syrups or suspensions, or they are presented as a dry product for constitution with water or other suitable vehicle before use. Such liquid preparations are prepared by conventional means with acceptable excipients or carriers such as suspending agents (e.g., sorbitol syrup cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). In some embodiments, the preparations optionally contain buffer salts, flavoring, coloring and sweetening agents as appropriate.

In some embodiments, topical compositions disclosed herein are in the form of a viscous liquid, solution, suspension, liposomal formulations, gel, jelly, cream, lotion, ointment, suppository, foam, aerosol spray aqueous or oily suspensions or solutions, emulsions, or emulsion ointments. In one embodiment, a topical composition is provided which includes a topical carrier. For example, thickeners, diluents, emulsifiers, dispersing aids or binders are optionally used as needed. The topical carrier is selected so as to provide the composition in the desired form, e.g., as a liquid, lotion, cream, paste, gel, powder, or ointment, and are comprised of a material of either naturally occurring or synthetic origin. Examples of suitable topical carriers for use herein include water, alcohols and other nontoxic organic solvents, glycerin, mineral oil, silicone, petroleum jelly, lanolin, fatty acids, vegetable oils, parabens, aloe vera, waxes, and the like. In some embodiments, topical formulations for application to skin include ointments, lotions, pastes, creams, gels, drops, suppositories, sprays, liquids, powders, shampoos, and transdermal patches.

In certain embodiments, ointments and creams are, for example, formulated with an aqueous or oily base with the addition of suitable thickening and/or gelling agents. Lotions are formulated with an aqueous or oily base and will in general also contain one or more emulsifying agents, stabilizing agents, dispersing agents, suspending agents, thickening agents, or coloring agents.

Any composition described herein optionally comprises one or more lubricant(s) such as, but not limited to, calcium tearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, Dow Corning 200® Fluid 100cs, a syloid silica gel, a coagulated aerosol of synthetic silica, or mixtures thereof.

In some embodiments, one function of the carrier is to enhance surface penetration of the active ingredients. Suitable carriers are well known to skilled practitioners, and include liposomes, ethanol, dimethylsulfoxide (DMSO), petroleum jelly (petrolatum), mineral oil (liquid petrolatum), water, dimethylformamide, dekaoxyethylene-oleylether, oleic acid, 2-pyrrolidone, and Azone® brand penetration enhancer (Upjohn).

In some embodiments, one or more nanoparticles are encapsulated in a liposome. In further or additional embodiments, a composition described herein comprises one or more nanoparticles encapsulated in a liposome and a moisturizing agent. In certain embodiments, the encapsulated nanoparticle provides for sustained release of the nanoparticle. In some embodiments, one or more microparticles are encapsulated in a liposome. In further or additional embodiments, a composition described herein comprises one or more microparticles encapsulated in a liposome and a moisturizing agent. In certain embodiments, the encapsulated microparticle provides for sustained release of the microparticle.

In one embodiment, the compositions are in a form suitable for cosmetic application including, but not limited to, lotions, ointments, creams, sprays, spritzes, aqueous or aqueous-alcoholic mixture gels, mousses, patches, pads, masks, moistened clothes, wipes, solid sticks, clear sticks, lip sticks, aerosol creams, anhydrous powders, talcs, tonics, oils, emulsions or bath salts.

In another embodiment, the composition optionally contains irritation-mitigating additives to minimize or eliminate the possibility of skin irritation or skin damage resulting from the chemical compound to be administered, or other components of the composition. Suitable irritation-mitigating additives include for example: α-tocopherol, monoamine oxidase inhibitors (e.g., 2-phenyl-1-ethanol), glycerin, salicylates, ascorbates, ionophores (e.g., monensin), amphiphilic amines, avenanthramides (e.g., SymCalmin® 143535), DragoCalm®, ammonium chloride, N-acetylcysteine, capsaicin, and/or chloroquine.

The “effective amount”, however, will take into account any toxicity effects that may occur, for example, severe skin irritation with higher doses of the active agents disclosed herein. Suggested endpoints may first be measured in vitro or in an animal model to determine the acceptable range of active agents to be used in conjunction with the compositions disclosed herein. The “effective amount” varies depending on the compound, the disease and its severity, and the age, weight, etc., of the subject to be treated.

In certain embodiments, the compositions and formulations disclosed herein comprises a nanoscale particle in a concentration of about 0.0000001%, about 0.0000005%, about 0.000001%, about 0.000002%, about 0.000004%, about 0.000006%, about 0.000008%, about 0.00001%, about 0.0001% about 0.001%, about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise a nanoscale particle in a concentration from about 0.0000001% to about 5%, from about 0.0000001% to about 1%, from about 0.000001% to about 1%, from about 0.0000001% to about 0.001%, from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise a microscale particle in a concentration of about 0.0000001%, about 0.0000005%, about 0.000001%, about 0.000002%, about 0.000004%, about 0.000006%, about 0.000008%, about 0.00001%, about 0.0001% about 0.001%, about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise a microscale particle in a concentration from about 0.0000001% to about 5%, from about 0.0000001% to about 1%, from about 0.000001% to about 1%, from about 0.0000001% to about 0.001%, from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise nanoscale titanium dioxide in a concentration of about 0.005%, about 0.008%, about 0.01%, about 0.05%, about 0.08%, about 0.1%, about 0.15%, about 0.175%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise nanoscale titanium dioxide in a concentration from about 0.005% to about 50%, from about 0.05% to about 5%, from about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise microscale titanium dioxide in a concentration of about 0.005%, about 0.008%, about 0.01%, about 0.05%, about 0.08%, about 0.1%, about 0.15%, about 0.175%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise microscale titanium dioxide in a concentration from about 0.005% to about 50%, from about 0.05% to about 5%, from about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise nanoscale titanium dioxide in an amount of about 0.01 mg/mL to about 3 mg/mL, about 0.02 mg/mL to about 3 mg/mL, about 0.03 mg/mL to about 3 mg/mL, about 0.04 mg/mL to about 3 mg/mL, about 0.05 mg/mL to about 3 mg/mL, about 0.06 mg/mL to about 3 mg/mL, about 0.07 mg/mL to about 3 mg/mL, about 0.08 mg/mL to about 3 mg/mL, about 0.09 mg/mL to about 3 mg/mL, about 0.1 mg/mL to about 3 mg/mL, about 0.01 mg/mL to about 2.9 mg/mL, about 0.01 mg/mL to about 2.8 mg/mL, about 0.01 mg/mL to about 2.6 mg/mL, about 0.01 mg/mL to about 2.5 mg/mL, about 0.01 mg/mL to about 2.4 mg/mL, about 0.01 mg/mL to about 2.3 mg/mL, about 0.01 mg/mL to about 2.2 mg/mL, about 0.01 mg/mL to about 2.1 mg/mL, about 0.01 mg/mL to about 2 mg/mL, about 0.01 mg/mL to about 1.9 mg/mL, about 0.01 mg/mL to about 1.8 mg/mL, about 0.01 mg/mL to about 1.7 mg/mL, about 0.1 mg/mL to about 2 mg/mL, about 0.5 mg/mL to about 2 mg/mL, about 1 mg/mL to about 3 mg/mL, about 1.5 mg/mL to about 1.9 mg/mL, about 0.1 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, or about 3 mg/mL.

In certain embodiments, the compositions and formulations disclosed herein comprise microscale titanium dioxide in an amount of about 0.01 mg/mL to about 3 mg/mL, about 0.02 mg/mL to about 3 mg/mL, about 0.03 mg/mL to about 3 mg/mL, about 0.04 mg/mL to about 3 mg/mL, about 0.05 mg/mL to about 3 mg/mL, about 0.06 mg/mL to about 3 mg/mL, about 0.07 mg/mL to about 3 mg/mL, about 0.08 mg/mL to about 3 mg/mL, about 0.09 mg/mL to about 3 mg/mL, about 0.1 mg/mL to about 3 mg/mL, about 0.01 mg/mL to about 2.9 mg/mL, about 0.01 mg/mL to about 2.8 mg/mL, about 0.01 mg/mL to about 2.6 mg/mL, about 0.01 mg/mL to about 2.5 mg/mL, about 0.01 mg/mL to about 2.4 mg/mL, about 0.01 mg/mL to about 2.3 mg/mL, about 0.01 mg/mL to about 2.2 mg/mL, about 0.01 mg/mL to about 2.1 mg/mL, about 0.01 mg/mL to about 2 mg/mL, about 0.01 mg/mL to about 1.9 mg/mL, about 0.01 mg/mL to about 1.8 mg/mL, about 0.01 mg/mL to about 1.7 mg/mL, about 0.1 mg/mL to about 2 mg/mL, about 0.5 mg/mL to about 2 mg/mL, about 1 mg/mL to about 3 mg/mL, about 1.5 mg/mL to about 1.9 mg/mL, about 0.1 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, or about 3 mg/mL.

In certain embodiments, the compositions and formulations disclosed herein comprise nanoscale titanium in a concentration of about 0.005%, about 0.008%, about 0.01%, about 0.05%, about 0.08%, about 0.1%, about 0.15%, about 0.175%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise nanoscale titanium in a concentration from about 0.005% to about 50%, from about 0.05% to about 5%, from about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, from about 0.05% to about 0.2%, or from about 0.005% to about 2% by weight relative to the total weight of the composition or formulation. In specific embodiments, the compositions and formulations disclosed herein comprise nanoscale titanium in a concentration of about 0.05%, 0.075%, 0.1%, 0.125%, 0.15%, 0.175%, 0.2%, 0.225%, 0.25%, 0.275%, or 0.3%.

In certain embodiments, the compositions and formulations disclosed herein comprise microscale titanium in a concentration of about 0.005%, about 0.008%, about 0.01%, about 0.05%, about 0.08%, about 0.1%, about 0.15%, about 0.175%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise microscale titanium in a concentration from about 0.005% to about 50%, from about 0.05% to about 5%, from about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, from about 0.05% to about 0.2%, or from about 0.005% to about 2% by weight relative to the total weight of the composition or formulation. In specific embodiments, the compositions and formulations disclosed herein comprise nanoscale titanium in a concentration of about 0.05%, 0.075%, 0.1%, 0.125%, 0.15%, 0.175%, 0.2%, 0.225%, 0.25%, 0.275%, or 0.3%.

In certain embodiments, the compositions and formulations disclosed herein comprise nanoscale titanium in an amount of about 0.01 mg/mL to about 3 mg/mL, about 0.02 mg/mL to about 3 mg/mL, about 0.03 mg/mL to about 3 mg/mL, about 0.04 mg/mL to about 3 mg/mL, about 0.05 mg/mL to about 3 mg/mL, about 0.06 mg/mL to about 3 mg/mL, about 0.07 mg/mL to about 3 mg/mL, about 0.08 mg/mL to about 3 mg/mL, about 0.09 mg/mL to about 3 mg/mL, about 0.1 mg/mL to about 3 mg/mL, about 0.01 mg/mL to about 2.9 mg/mL, about 0.01 mg/mL to about 2.8 mg/mL, about 0.01 mg/mL to about 2.6 mg/mL, about 0.01 mg/mL to about 2.5 mg/mL, about 0.01 mg/mL to about 2.4 mg/mL, about 0.01 mg/mL to about 2.3 mg/mL, about 0.01 mg/mL to about 2.2 mg/mL, about 0.01 mg/mL to about 2.1 mg/mL, about 0.01 mg/mL to about 2 mg/mL, about 0.01 mg/mL to about 1.9 mg/mL, about 0.01 mg/mL to about 1.8 mg/mL, about 0.01 mg/mL to about 1.7 mg/mL, about 0.1 mg/mL to about 2 mg/mL, about 0.5 mg/mL to about 2 mg/mL, about 1 mg/mL to about 3 mg/mL, about 1.5 mg/mL to about 1.9 mg/mL, about 0.1 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, or about 3 mg/mL.

In certain embodiments, the compositions and formulations disclosed herein comprise microscale titanium in an amount of about 0.01 mg/mL to about 3 mg/mL, about 0.02 mg/mL to about 3 mg/mL, about 0.03 mg/mL to about 3 mg/mL, about 0.04 mg/mL to about 3 mg/mL, about 0.05 mg/mL to about 3 mg/mL, about 0.06 mg/mL to about 3 mg/mL, about 0.07 mg/mL to about 3 mg/mL, about 0.08 mg/mL to about 3 mg/mL, about 0.09 mg/mL to about 3 mg/mL, about 0.1 mg/mL to about 3 mg/mL, about 0.01 mg/mL to about 2.9 mg/mL, about 0.01 mg/mL to about 2.8 mg/mL, about 0.01 mg/mL to about 2.6 mg/mL, about 0.01 mg/mL to about 2.5 mg/mL, about 0.01 mg/mL to about 2.4 mg/mL, about 0.01 mg/mL to about 2.3 mg/mL, about 0.01 mg/mL to about 2.2 mg/mL, about 0.01 mg/mL to about 2.1 mg/mL, about 0.01 mg/mL to about 2 mg/mL, about 0.01 mg/mL to about 1.9 mg/mL, about 0.01 mg/mL to about 1.8 mg/mL, about 0.01 mg/mL to about 1.7 mg/mL, about 0.1 mg/mL to about 2 mg/mL, about 0.5 mg/mL to about 2 mg/mL, about 1 mg/mL to about 3 mg/mL, about 1.5 mg/mL to about 1.9 mg/mL, about 0.1 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, or about 3 mg/mL.

In certain embodiments, the compositions and formulations disclosed herein comprise nanoscale colloidal silver in a concentration of about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise nanoscale colloidal silver in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise microscale colloidal silver in a concentration of about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise microscale colloidal silver in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise nanoscale silver salt in a concentration of about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise nanoscale silver salt in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise microscale silver salt in a concentration of about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise microscale silver salt in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise silver in a concentration of about 0.0000001%, about 0.0000002%, about 0.0000004%, about 0.0000006%, about 0.0000008%, about 0.000001%, about 0.000002%, about 0.000004%, about 0.000006%, about 0.000008%, about 0.00001%, about 0.00002%, about 0.00004%, about 0.00006%, about 0.00008%, about 0.0001%, about 0.0002%, about 0.0004%, about 0.0006%, about 0.0008%, about 0.001%, about 0.002%, about 0.004%, about 0.006%, about 0.008%, about 0.01%, about 0.02%, about 0.04%, about 0.06%, about 0.08%, or about 0.1%, by weight relative to the total weight of the composition or formulation. Preferably, the compositions and formulations disclosed herein comprise silver in a concentration from about 0.0000001% to about 5%, from about 0.0000001% to about 1%, from about 0.000001% to about 1%, from about 0.0000001% to about 0.01%, from about 0.0000001% to about 0.001%, from about 0.0000002% to about 0.001%, from about 0.0000004% to about 0.001%, from about 0.0000006% to about 0.001%, from about 0.0000008% to about 0.001%, from about 0.000001% to about 0.001%, from about 0.000002% to about 0.001%, from about 0.000004% to about 0.001%, from about 0.000006% to about 0.001%, from about 0.000008% to about 0.0001%, or from about 0.000008% to about 0.0001% by weight relative to the total weight of the composition or formulation.

In certain embodiments, the compositions and formulations disclosed herein comprise silver in an amount of about 0.001 mg/mL to about 0.1 mg/mL, about 0.002 mg/mL to about 0.1 mg/mL, about 0.004 mg/mL to about 0.1 mg/mL, about 0.006 mg/mL to about 0.1 mg/mL, about 0.008 mg/mL to about 0.1 mg/mL, about 0.01 mg/mL to about 0.1 mg/mL, about 0.02 mg/mL to about 0.1 mg/mL, about 0.04 mg/mL to about 0.1 mg/mL, about 0.06 mg/mL to about 0.1 mg/mL, about 0.001 mg/mL to about 0.08 mg/mL, about 0.001 mg/mL to about 0.06 mg/mL, about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL, about 0.009 mg/mL, about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, or about 0.15 mg/mL.

In certain embodiments, the compositions and formulations disclosed herein comprise nanoscale zinc oxide in a concentration of about 0.00005%, about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise nanoscale zinc oxide in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprise microscale zinc oxide in a concentration of about 0.00005%, about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprise microscale zinc oxide in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In some embodiments, the compositions and formulations described herein comprise a film-forming polymer in an amount from about 0.05% to 50%, from about 0.05% to about 25%, from about 0.05% to about 20%, from about 0.05% to about 15%, from about 0.05% to about 10%, from about 0.05% to about 5%, from about 0.05% to about 2%, from about 0.05% to about 1%, from about 0.1% to about 10%, from about 0.1% to about 5%, from about 0.1% to about 3%, from about 0.1% to about 2%, from about 0.1% to about 1%, from about 1% to about 20%, from about 1% to about 30%, from about 1% to about 40%, from about 1% to about 50% by weight relative to the total weight of the composition. In some embodiments, the compositions and formulations described herein comprise a film-forming polymer in an amount in an amount of about 0.005%, about 0.008%, about 0.01%, about 0.05%, about 0.1%, about 0.2%, about 0.25%, about 0.3%, about 0.4%, about 0.45%, about 0.5%, about 0.55%, about 0.6%%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise polyolprepolymer-2 in an amount from about 0.05% to 50%, from about 0.05% to about 25%, from about 0.05% to about 20%, from about 0.05% to about 15%, from about 0.05% to about 10%, from about 0.05% to about 5%, from about 0.05% to about 2%, from about 0.05% to about 1%, from about 0.1% to about 10%, from about 0.1% to about 5%, from about 0.1% to about 3%, from about 0.1% to about 2%, from about 0.1% to about 1%, from about 1% to about 20%, from about 1% to about 30%, from about 1% to about 40%, from about 1% to about 50% by weight relative to the total weight of the composition. In some embodiments, the compositions and formulations described herein comprise polyolprepolymer-2 in an amount in an amount of about 0.005%, about 0.008%, about 0.01%, about 0.05%, about 0.1%, about 0.2%, about 0.25%, about 0.3%, about 0.4%, about 0.45%, about 0.5%, about 0.55%, about 0.6%%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise an iodine source in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise an iodine source in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise povidone-iodine in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise povidone-iodine in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise chlorhexidine-alcohol in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise povidone-iodine in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise a surfactant in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise a surfactant in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise benzalkonium chloride in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise benzalkonium chloride in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In certain embodiments, the compositions and formulations disclosed herein comprise a moisturizer in a concentration of about 0.5% to about 15%, about 5% to about 15%, about 10% to about 25%, about 10% to about 50%, about 10% to about 75%, about 10% to about 95%, about 0.5% to about 95%, about 5% to about 75%, about 15% to about 75%, about 25% to about 75%, about 50% to about 75%, about 15% to about 25%, about 15% to about 50%, about greater than 1%, greater than 5%, greater than 10%, greater than 20%, greater than 50%, less than about 90%, less than about 80%, less than about 70%, less than about 60%, less than about 50%, less than about 40%, less than about 30%, less than about 25%, less than about 20%, less than about 15%, less than about 10%, less than about 5%, about 0.05%, about 0.1%, about 0.5%, about 1% about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, about 95%, or about 98% by weight relative to the total weight of the composition or formulation.

### Administration

Certain embodiments describe a composition that is applied to a surface to provide a temporary and/or sustained disinfection of the surface.

Examples of routes of administration to a mammal include, but are not limited to, oral, buccal, inhalation, intradermal, subcutaneous, transmucosal, transdermal, or topical administration. Topical administration may also involve the use of transdermal administration such as transdermal patches or iontophoresis devices. The construction and use of transdermal patches for the delivery of pharmaceutical agents is well known in the art. See, e.g., U.S. Pat. Nos. 5,023,252, 4,992,445, and 5,001,139. Such patches are be constructed for continuous, pulsatile, or on demand delivery of pharmaceutical agents.

In some embodiments, any of the compositions and formulations described herein are used in combination with another agent. In some embodiments, a composition described herein and an additional agent are administered to a surface simultaneously. In other embodiments, a composition described herein and an additional agent are administered at staggered times. In some embodiments, a composition described herein and an additional agent are mixed together prior to application.

In some embodiments, any of the compositions and formulations described herein are heated prior to administration to a surface. In other embodiments, the compositions and formulations described herein are heated after administration to a surface. In some embodiments, the compositions and formulations described herein are heated to 40° C., 50° C., 60° C., 70° C., 80° C., 90° C., 100° C., 125° C., 150° C., 175° C., or 200° C. In some instances, heating the composition or formulation enhances the antimicrobial activity.

In some embodiments, any of the compositions and formulations described herein are not heated prior to administration to a surface. In other embodiments, the compositions and formulations described herein are not heated after administration to a surface. In some instances, not heating the composition or formulation enhances the antimicrobial activity.

In some embodiments, any of the compositions or formulations described herein are applied as needed or alternatively as a part of a disinfecting routine. In some embodiments, the composition is applied to a surface once, twice or three times daily. In other embodiments, the composition is applied to a surface once weekly, monthly, or yearly. In additional or further embodiments, the composition is applied to a surface 2, 3, 4, or 5 times weekly. In certain embodiments, the composition is applied to a surface every 3 hours, 6 hours, 12 hours, 24 hours, 36 hours, 48 hours, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

### EXAMPLES

The examples described herein are provided for illustrative purposes and are not to be understood as limitations of the scope of the described embodiments. The use of laboratory C. difficile strains and ribotypes include but are not limited to BI/NAP1/027, VPI 10463 (ATCC 43255), 630 (ATCC BAA-1382), VPI 11186 (ATCC 700057), 1351 (ATCC 43593), 4811 (ATCC 43602), 5036 (ATCC 43603), Ribotype 001 and ribotypes 106, 78 and 017 as described herein.

### Example 1

### Spore Preparation for C. Difficile

C. difficile cultures (10 ml) are prepared by overnight growth at 37° C. in TGY-vegetative medium (3% tryptic soy broth, 2% glucose, 1% yeast extract, 0.1% 1-cysteine). Sporulating cultures were prepared by inoculating 0.6 ml TGY starter culture into 10 ml of each medium, followed by incubation for 24 h at 37° C. C. difficile spores were routinely prepared using DS medium.

For spore purification, spore suspensions are prepared in 600 ml DS medium. Spores are cleaned of debris by repeated centrifugation and washing with sterile distilled water, and are resuspended in distilled water at OD₆₀₀˜6 and stored at −20° C. until use. All spore preparations are >99% free of sporulating cells, cell debris and germinated spores, as determined by phase-contrast microscopy.

### Example 2

### Germination

Under aerobic conditions, C. difficile spore germination is non-heat activated at 37° C. for 5 min. Consequently, all germination experiments described herein are heat-activated spores unless noted otherwise. After non-heat activation, spores are sonicated briefly to break up any clumps and incubated at 37° C. for 5 min before addition of germinants, and the OD₆₀₀ of the spore suspensions is measured to assess spore germination (Smartspec 3000 Spectrophotometer, Bio-Rad Laboratories); levels of spore germination are also confirmed by phase-contrast microscopy.

Under aerobic conditions, C. difficile spore germination is heat activated at 80° C. for 10 min. Consequently, all germination experiments described herein are heat-activated spores unless noted otherwise. After heat activation, spores are cooled to room temperature, sonicated briefly to break up any clumps and incubated at 40° C. for 10 min before addition of germinants, and the OD₆₀₀ of the spore suspensions is measured to assess spore germination (Smartspec 3000 Spectrophotometer, Bio-Rad Laboratories); levels of spore germination are also confirmed by phase-contrast microscopy.

### Method A

Germination in nutrient medium is performed in BHI broth. Germination with bile salts and/or glycine is carried out in 10 mM Na₂HPO₄ buffer (pH 7.5) to reduce the background germination caused by Pi. Germination is routinely carried out aerobically in 25 mM sodium phosphate buffer (pH 7.5) unless noted otherwise. The extent of spore germination is determined by measuring the decrease in OD₆₀₀ of germinating spore suspensions, and is expressed as a percentage of the initial OD₆₀₀. Since a decrease in OD₆₀₀ of ˜65% corresponds to ≧99% spore germination as assessed by phase-contrast microscopy, the percentage decrease in the OD₆₀₀ is converted to the percentage germination by taking an OD₆₀₀ decrease of 65% as 100% germination. The rate of germination is expressed as the maximum rate of loss of OD₆₀₀ of spore suspensions relative to initial values. To evaluate effects of pH on spore germination rates, germination is performed in 25 mM sodium citrate buffer (pH 2 and 4), 25 mM sodium phosphate buffer (pH 2, 5 and 7.5) or 25 mM Tris/HCl buffer (pH 8.5) at 37° C. All values reported are averages of two experiments performed with two independent spore preparations, and individual values vary by ≦15% from the average.

### Method B

For germination with dodecylamine, spores at OD₆₀₀˜1 are used without heat activation. Spores are incubated at 37° C. with 1 mM dodecylamine in 25 mM Tris/HCl buffer (pH 7.4). Aliquots (1 ml) of germinating cultures were centrifuged at 16110 g for 2 min in a microcentrifuge, and DPA in the supernatant fluid was measured by monitoring OD₂₇₀. The total OD₂₇₀ that are released from these spores is determined by boiling a sample of dormant spores at an OD₆₀₀ of 1 for 60 min, followed by cooling on ice, centrifugation and measurement of the OD₂₇₀ of the supernatant fluid. All experiments with dodecylamine are repeated at least twice, and results for different experiments differ by ≦5%.

### Method C

For germination with Ca-DPA, spores are germinated with or without prior heat activation, cooled to room temperature, diluted to OD₆₀₀˜1.5 and incubated at 37° C. with Ca-DPA (50 mM CaCl2, 50 mM DPA adjusted to pH 8.0 with Tris/HCl). At various times, 1 ml aliquots are centrifuged for 2 min in a microcentrifuge, and the spore pellet is washed four times with sterile distilled water and suspended in 1 ml sterile water. Residual spore core DPA content is determined by boiling samples for 60 min, centrifuging them for 5 min, and measuring the OD₂₇₀ of the supernatant fluid. The change in the OD₆₀₀ of spore cultures during germination with Ca-DPA is also measured as described above. All experiments with Ca-DPA are repeated at least twice, and results for different experiments differ by ≦5%.

### Example 3

### Germinating Solutions for C. Difficile

Exemplary solutions found to be effective in germinating Clostridium Difficile are given below:

| | Concentration |
|---|---|
| Component | (mg/L) |
| Amino acid | |
| Histidine | 100 |
| Trytophan | 100 |
| Glycine | 100 |
| Tyrosine | 100 |
| Arginine | 200 |
| Phenylalanine | 200 |
| Methionine | 200 |
| Threonine | 200 |
| Alanine | 200 |
| Lysine | 300 |
| Serine | 300 |
| Valine | 300 |
| Isoleucine | 300 |
| Aspartic acid | 300 |
| Leucine | 400 |
| Cysteine | 500 |
| Proline | 600 |
| Glutamic acid | 900 |
| Mineral | |
| KH₂PO₄ | 300 |
| Na₂HPO₄ | 1500 |
| NaCl | 90 |
| CaCl₂•2H₂O | 26 |
| MgCl₂•6H₂O | 20 |
| MnCl₂•4H₂O | 10 |
| (NH₄)₂SO₄ | 40 |
| FeSO₄•7H₂O | 4 |
| CoCl₂•6H₂O | 1 |
| NaHCO₃ | 5000 |
| Bile salt | |
| Taurocholic acid | 1000 |

### Example 4

### Synthesis of Microscale Particles

Silver oxide powder having an average particle size of 0.5 um is prepared as the raw material powder. The raw material powder is sprayed together with a carrier gas (Ar) to RF plasma, and oxygen is flown as a reaction gas at a flow rate of 80 L/min. At which time, the pressure in the reaction vessel is adjusted to a low pressure of 25 kPa. Thus, the silver oxide microparticles are produced through a sublimation process to execute an oxidation reaction while sublimating the raw material powder. An average particle size (D50), a specific surface area, and a crystalline structure of the obtained silver oxide microparticles are measured and evaluated.

### Example 5

### Synthesis of Nanoscale Particles

Silver oxide powder having an average particle size of 100 nm is prepared as the raw material powder. The raw material powder is sprayed together with a carrier gas (Ar) to RF plasma, and Argon is flown as a reaction gas at a flow rate of 40 L/min and air is flown at a flow rate of 40 L/min. At which time, the pressure in the reaction vessel is adjusted to a pressure of 40 kPa. Thus, the silver oxide microparticles are produced through a sublimation process to execute an oxidation reaction while sublimating the raw material powder. The resulting silver oxide nanoparticles undergo a heat treatment process under atmospheric pressure at about 500° C. to 900° C. for 1 to 2 hours. An average particle size (D50), a specific surface area, and a crystalline structure of the obtained silver oxide microparticles are measured and evaluated.

### Example 6

### Nanoscale Particle Antimicrobial Composition

Exemplary antimicrobial compositions (on a weight percent basis, based on the total weight of the composition) are given below with water/aqua/eau constituting the balance of the composition.

| | | | | C | D |
|---|---|---|---|---|---|
| | Ranges | A | B | % | % |
| Component | % (w/w) | % (w/w) | % (w/w) | (w/w) | (w/w) |
| Ethyl Alcohol | 40-75 | 62 | 58 | 60 | 55 |
| Polyquaternium-37 | 1-6 | 3 | 3 | 2.8 | 2.5 |
| PPG-12/SMDI | 0.01-5 | 0.75 | 0.01 | 1.1 | 0.75 |
| Copolymer | | | | | |
| Pentylene Glycol | 0.001-3.5 | 0.5 | 0.5 | 0.6 | 0.5 |
| Butylene Glycol | 0.001-3.5 | 0.5 | 0.5 | 0.5 | 0.5 |
| Leptospermum | 0.001-3 | 0.15 | 0.2 | 0.15 | 0.18 |
| Petersonii Oil | | | | | |
| Cetrimonium Chloride | 0.005-2 | 0.3 | 0.3 | 0.3 | 0.3 |
| Titanium Dioxide | 0.005-1.75 | 0.2 | — | 0.25 | 0.2 |
| Nanoparticle | | | | | |
| Benzalkonium | 0.005-2.5 | 0.2 | 0.2 | 0.2 | 0.5 |
| Chloride | | | | | |
| Hydroxyphenyl | 0.005-2.5 | 0.5 | 0.5 | 0.5 | 0.5 |
| Propamidobenzoic | | | | | |
| Acid | | | | | |
| Yellow 5 | 0.001-0.1 | 0.05 | 0.04 | 0.03 | 0.03 |
| Colloidal Silver | 0.01-1.5 | 0.1 | 0.25 | — | — |
| Source Base Product | | | | | |
| Silver citrate | 0.5-30 | — | — | — | 10 |
| Silver-copper alloy | 0.05-15 | — | — | — | 5 |
| nanopowder | | | | | |
| Citric acid | 0.005-20 | — | — | — | 10 |

### Example 7

### Nanoscale Particle Antimicrobial Composition

Exemplary antimicrobial compositions (on a weight percent basis, based on the total weight of the composition) are given below.

| | | | | C | D |
|---|---|---|---|---|---|
| | Ranges | A | B | % | % |
| Component | % (w/w) | % (w/w) | % (w/w) | (w/w) | (w/w) |
| Deionized Water | 15-85% | 20.14 | 30.33 | 82.61 | 82.5 |
| Yellow 5 - 1% | | 0.95 | 0.90 | 0.95 | 0.90 |
| solution* | | | | | |
| Elemental silver | 3.8-4.8 | 4 | 4.5 | 4 | 4.8 |
| nanoparticle (100 nm) | | | | | |
| Titanium Dioxide | 0-6 | — | — | 5 | 4.3 |
| nanoparticle | | | | | |
| Titanium dioxide USP | 2-3 | 2.5 | 2.7 | 2.5 | 2.4 |
| #3328 | | | | | |
| SD Alcohol SDA 40-2 | 0-70 | 67.2 | 57.01 | — | — |
| (190 Proof) | | | | | |
| SymCalmin ® No. | 0.5-1.5 | 1 | 1.06 | 1 | 0.8 |
| 143535 | | | | | |
| Cosmedia ® Ultragel | 0.5-2.5 | 2 | 1.5 | 0.95 | 1.2 |
| 300 | | | | | |
| Euxyl ® PE 9010 | 0-2 | — | — | 1 | 1.3 |
| PPG-12/SMDI | 1.5-2.5 | 1.75 | 1.8 | 1.75 | 1.5 |
| Copolymer | | | | | |
| Stepanquat ® 50 NF | 0-0.4 | 0.26 | 0.1 | — | — |
| Lemon Tea Tree Oil | 0.1-0.3 | 0.2 | 0.1 | 0.2 | 0.25 |
| AMP ® Ultra PC | 0.01-0.07 | — | — | 0.04 | 0.05 |
| 2000 | | | | | |

*1% solution comprises 89% deionized water, 10% incroquat, 1% FD&C Yellow 5

### Example 8

### Nanoscale Particle Antimicrobial Moisturizer

Exemplary antimicrobial moisturizers (on a weight percent basis, based on the total weight of the composition) are given below.

| | | | | C | D |
|---|---|---|---|---|---|
| | Ranges | A | B | % | % |
| Component | % (w/w) | % (w/w) | % (w/w) | (w/w) | (w/w) |
| Deionized Water | 55-65 | 61.14 | 61.57 | 59.77 | 61.05 |
| Structure ® Solanace | 0.5-1.5 | 1 | 0.9 | 1.2 | 0.8 |
| 28-1808 | | | | | |
| Elemental silver | 3.5-4.5 | 4 | 4.2 | 3.8 | 4.1 |
| nanoparticle (100 nm) | | | | | |
| Keltrol CG-RD | 0.15-0.25 | 0.2 | 0.2 | 0.18 | 0.2 |
| Glycerin USP 99.7% | 2-4 | 3 | 2.8 | 3 | 3.2 |
| D-Panthenol 75L | 0.5-2 | 1 | 0.9 | 0.8 | 1 |
| Potassium Sorbate | 0.05-0.3 | 0.1 | 0.1 | 0.2 | 0.15 |
| PDR FCC K | | | | | |
| EuxyL ® PE 9010 | 0.5-1.5 | 1 | 1 | 1.1 | 0.8 |
| Emulium ® Kappa | 3.8-4.8 | 4 | 4.2 | 4.4 | 4 |
| Cetyl Alcohol C16-98, | 2-3 | 2.5 | 2.6 | 2.4 | 2.5 |
| NF Grade | | | | | |
| Cutina ® CP | 1.8-2.5 | 2 | 2 | 2.3 | 2.2 |
| Dermofeel ® TC-7 | 3.5-6 | 5 | 4 | 5 | 4.5 |
| Hydromide ® Blend | 1.5-2.5 | 2 | 2 | 2.1 | 2.1 |
| Net Sterol-100 | 0.25-1 | 0.5 | 0.5 | 0.5 | 0.6 |
| Jojoba Oil Golden | 0.5-1.8 | 1 | 1.5 | 0.9 | 1 |
| High Oleic Sunflower | 1-3 | 2 | 1.5 | 1.8 | 2 |
| Oil | | | | | |
| Dow Corning 200 ® | 1-3 | 2 | 2.1 | 2.3 | 1.8 |
| Fluid 100 cs | | | | | |
| Vitamin E Acetate | 0.05-0.3 | 0.1 | 0.1 | 0.1 | 0.2 |
| USP/FCC | | | | | |
| Liponate ® GC | 3-4.5 | 3.5 | 3.5 | 3.7 | 3.5 |
| DragoCalm ® | 1.5-3 | 2 | 2.3 | 2.4 | 2.1 |
| PPG-12/SMDI | 1-3 | 1.5 | 1.6 | 1.5 | 1.8 |
| Copolymer | | | | | |
| Stepanquat ® 50 NF | 0.1-0.5 | 0.26 | 0.25 | 0.3 | 0.2 |
| Lavender Royale “B” | 0.1-0.4 | 0.2 | 0.18 | 0.25 | 0.2 |

### Example 9

### Nanoscale Particle Antimicrobial Foam Soap

Exemplary antimicrobial compositions (on a weight percent basis, based on the total weight of the composition) are given below.

| | | | | C | D |
|---|---|---|---|---|---|
| | Ranges | A | B | % | % |
| Component | % (w/w) | % (w/w) | % (w/w) | (w/w) | (w/w) |
| Deionized Water | 70-80 | 76.390 | 78.5 | 76.32 | 75.75 |
| 1,3-Butylene glycol | 2.2-3.5 | 3 | 2.5 | 2.8 | 2.6 |
| BRIJ ® IC20-70 | 0.5-2 | 1.25 | 1 | 1.3 | 1.5 |
| Euxyl ® PE 9010 | 0.3-2 | 1 | 1 | 1.3 | 0.9 |
| Lemon Tea Tree Oil | 0-0.5 | 0.1 | 0.15 | 0.1 | 0.1 |
| Incromine ® Oxide C | 8-14 | 12 | 10 | 12 | 13 |
| Arlasilk ® PTC | 1.4-2.5 | 2 | 2.3 | 2 | 1.8 |
| Stepanquat ® 50 NF | 0.1-0.3 | 0.26 | 0.25 | 0.28 | 0.25 |
| Elemental silver | 3.5-4.8 | 4 | 4.3 | 3.9 | 4.1 |
| nanoparticle (100 nm) | | | | | |

### Example 10

### Stability of Nanoscale Particle Antimicrobial Compositions

Compositions 1A-D, 2A-D, 3A-D, and 4A-D are monitored over time to assess the stability of the formulations against nanoparticle aggregation. Stability is measured using optical spectroscopy to measure changes in solution turbidity over time. Dynamic light scattering (DLS) is used to measure changes in the hydrodynamic diameter of the aggregates over time, providing information on the size of the aggregates present. Turbidity and DLS measurements of the compositions are conducted at 0° C., 5° C., 15° C., 25° C., 35° C., 50° C., and 75° C.

### Example 11

### Film Morphology and Particle Distribution of Nanoscale Particle Antimicrobial Compositions

Compositions 1A-D, 2A-D, 3A-D, and 4A-D are sprayed onto glass slides positions at various locations within the spray pattern. Optical microscopy is used to examine the morphology of the polymer/nanoparticle film. Bright field measurements are used to analyze the gross morphology of the polymer film. Dark field measurements are used to measure the distribution of silver nanoparticles within the film.

### Example 12

### Ion Release Rates of Nanoscale Particle Antimicrobial Compositions

Compositions 1A-D, 2A-D, 3A-D, and 4A-D are coated on several substrates and allowed to dry to form a film. A baseline for the initial amount of silver present in the films is established with digestion of a film with nitric acid, followed by analysis of silver content using inductively coupled plasma mass spectroscopy (ICP-MS) or atomic absorption spectroscopy (AAS). The remaining films are aged, undisturbed, for varying amounts of time. At each time point (e.g., 1 h, 2 h, 3 h, 4 h, 5 h, 6 h, 12 h, 24 h, 36 h, 48 h, and 1 week), a portion of the film is dissolved into water using sonication and agitation. The resultant solution is passed through an ultrafine filer to remove undissolved nanoparticles. The solution is treated with nitric acid to separate bound silver ions from any polymer and the silver concentration is measured using ICP-MS analysis. Dissolution kinetics are obtained by analysis of the data at different time points.

### Example 13

### Nanoparticle Association Assay

A panel of representative bacteria is utilized to determine the amount of binding of nanoparticles to spores as a function of nanoparticle size, surface and suspension media. Long-term antimicrobial efficacy is achieved by associating dormant spores with an effectively dormant nanoparticle. Under sporulation conditions, the silver nanoparticles release silver ions that disrupt growth. Spores are boiled and the spore concentration is determined using microscopy. A known concentration of spores is mixed with a known concentration of nanoparticles. The mixture is allowed to incubate for 5 minutes. The spores and nanoparticles are separated via filtration. The resulting pool of spores is analysed using TEM to determine the number of nanoparticles bound to each spore.

### Example 14

### Disk Diffusion Assay

A panel of representative bacteria is utilized to evaluate the efficacy of the antimicrobial formulations. Cultures of Pseudomonas aerugenosa, Clostridium perfingens, Clostridium difficile and Staphylococcus aureus are obtained from American Type Culture Collection. Screening is accomplished using a disk diffusion assay to provide a qualitative means of comparing the release of silver ions and anti-bacterial properties of thin-films of nanoparticles. Plates are prepared containing a nutrient agar for the growth of the bacteria, followed by the uniform application of bacterial culture. Thin film samples of Examples 1A, 2A and 2C are prepared by filtering nanoparticle solutions through filter paper, drying the filter paper, and punching small coupons from the dried paper. The mass concentration of silver in the samples is determined by digesting the silver nanoparticles from a coupon in nitric acid solution, followed by quantitative measurement of the silver ion concentration using inductively coupled plasma mass spectroscopy (ICP-MS) or atomic absorption spectroscopy (AAS). The thin film samples of Examples 1A, 2A and 2C are placed into the cultured plate. The plates are incubated at 37° C. for 24 hours. Following incubation, the samples are analyzed for inhibition zones where bacteria are killed.

### Example 15

### In Vitro Efficacy of the Nanoscale Particle Antimicrobial Composition 1A

Minimum inhibitory concentration studies are performed using the gram-negative enterobacterium Pseudomonas aerugenosa (American Type Culture Collection #9027) in accordance with the protocol for testing the bactericidal activity of antimicrobial agents (Document M26-T of the National Center for Clinical and Laboratory Standards). P. aerugenosa are cultured overnight at 37° C. in trypsin soy broth to a final density of approximately 1×10⁸ cfu/ml (0.5 McFarland standard) and then diluted 1:10 with cation-adjusted Mueller-Hinton medium. 10 microliters of this bacterial culture are added to 200 microliters of an already-prepared dilution series of the test antimicrobial solution comprising Composition 1A. After a 5 minute incubation at room temperature, 10 microliters of wash test solution are plated onto a sector of a Letheen-agar plate and incubated at 37° C. overnight. MIC breakpoint is interpreted as the highest dilution for which no growth was evident.

### Example 16

### Efficacy of the Nanoscale Particle Antimicrobial Composition 1A and 2A on Article Surface

Duplicate samples of two silicone catheters, one coated with the Nanoscale Particle Antimicrobial Composition 1A, one coated with Composition 2A and one uncoated control, are cut into 2-cm length samples and each are placed in a separate sterile tube. Inoculum cultures of 1×10⁵ E. coli cells/mL (clinical isolate from UTI) in synthetic urine are prepared. 1 mL of synthetic urine solution and 1 mL inoculum culture are added to each tube containing the catheter samples, and the tubes are incubated at 37° C., rotating at 20 rpm. (Day 0)

Upon completion of incubation time (Days 1, 4), the following assays are performed on separate duplicate samples for each day of incubation: (1) planktonic growth of the contacting solution (CS); and (2) counting of attached viable cells (biofilm) on the catheter pieces (S). The dilutions tested were: 1:10, 10², 10³, 10⁴. On day 1, the S and CS solutions are plated in parallel onto MacConkey plates.

Duplicate samples of 5 different coated catheters, and one uncoated control catheter, are cut into 2-cm length samples and each is placed in a separate sterile tube. An inoculum culture of 1×10⁵ E. coli cells/mL (clinical isolate from UTI) in synthetic urine is prepared. 1 mL of the synthetic urine solution and 1 mL of the inoculum culture are added to each tube containing the catheter samples and the tubes are incubated at 37° C., rotating at 20 rpm (Day 0). Upon completion of the incubation times (Days 1, 2, 4, and 7), the following assays are performed on separate duplicate samples for each day of incubation: (a) planktonic growth of the contacting solution (CS); and (b) counting of attached viable cells (biofilm) on the catheter pieces (S). On Day 4, only the contacting solution is assayed. The catheter samples are not sonicated, but rather are transferred to new test tubes with a fresh challenge of 1×10⁵ E. coli cells. After an additional 3 days (which corresponded to Day 7 of the overall experiment), these samples are assayed for planktonic growth of the contacting solution (CS) and for attached viable cells (biofilm) (S).

### Example 17

### Efficacy of the Nanoscale Particle Antimicrobial Compositions on Article Surfaces

Small test objects with surfaces of stainless steel, ceramic tile, glass or paint are obtained. Four sets of the test objects of each material are exposed to Pseudomonas aerugenosa, Clostridium perfingens, Clostridium difficile or Staphylococcus aureus, then sprayed with Antimicrobial Composition 1A, 1B, 1C, 1D, 2A, 2B, 2C, 2D, 3A, 3B, 3C, 3D, 4A, 4B, 4C, and/or 4D. Another four sets of test objects of each material are sprayed with the antimicrobial composition. After the composition has dried, the test objects are exposed to Pseudomonas aerugenosa, Clostridium perfingens, Clostridium difficile or Staphylococcus aureus. After 6 h, the objects are placed contaminated side-down onto plates containing bacteria growth medium, then incubated at 37° C. for 12 h to allow any viable bacteria to grow. The presence or absence of bacterial growth indicates the efficacy of the composition on each surface.

### Example 18

### Efficacy of the Nanoscale Particle Antimicrobial Composition

The Nanoscale Particle Antimicrobial Compositions 1A, 2A, and 2C are tested against the avian influenza virus, Type A (H9N22), Turkey/Wis/66; SPAFAS through injection into embryonated chicken eggs. A virus suspension control is used for comparison purposes.

### Example 19

### Antimicrobial Treatment

Human Clinical Trial of the Safety and Efficacy of Nanoscale particle Antimicrobial Composition 1A, 2A or 2C to Prevent Recurrent Methicillin-Resistant Staphylococcus aureus (MRSA) Infection

Objective: The primary objective of this study is to evaluate the safety, local tolerability and efficacy of the nanoscale particle antimicrobial composition 1A, 2A or 2C. The composition is applied topically to subjects who are carriers of colonies of MRSA and MSSA. The extent of systemic absorption of the nanoscale particle antimicrobial composition is evaluated and the effect of the composition to clear colonies of MRSA/MSSA.

Study Design: This study is a randomized, double-blind, placebo-controlled, ascending dose Phase I/IIa study to evaluate the safety, tolerability and efficacy of topical nanoscale particle antimicrobial composition 1A, 2A or 2C in subjects colonized with methicillin-resistant/-sensitive Staphylococcus aureus (MRSA/MSSA). The first group of subjects receives the 1% nanoscale particle antimicrobial composition or placebo, the second group of subjects receives the 3% nanoscale particle antimicrobial composition or placebo, and the third group of subjects receives the 5% nanoscale particle antimicrobial composition or placebo. Dose escalation is performed after a brief safety evaluation of the tolerability after application of the nanoscale particle antimicrobial composition/placebo vehicle for three days. Pharmacokinetic samples are collected. Subjects are followed until 9 weeks after initiation of treatment.

Primary Outcome Measurements: explore safety and local tolerability and efficacy of nanoscale particle antimicrobial composition when applied topically to skin of subjects with colonized MRSA/MSSA; determine the extent of systemic absorption of nanoscale particle antimicrobial composition when applied to the skin of subjects. Secondary Outcome Measurements: to evaluate recurrence of MRSA/MSSA during the observation period (week 2 and week 9 after treatment).

### Example 20

### Antimicrobial Treatment

Human Clinical Trial of the Safety and Efficacy of Nanoscale particle Antimicrobial Composition 1A, 2A or 2C

Objective: The primary objective of this study is to measure the antimicrobial effectiveness of the nanoscale particle antimicrobial composition that live on the surface of the skin.

Study Design: This Phase III study is a non-randomized, single-center, open-label study. The study is comprised of 2 parts with approximately 20 subjects participating in each part. Subjects eligible for Part 1 have the nanoscale particle antimicrobial composition 1A, 2A or 2C applied to 6 sites across the chest and/or abdomen and chlorhexidine 2% solution (control, FDA approved medication) applied to 6 matching sites on the contralateral side. Swab cultures are obtained at specified time points over a period of 3 days. Subjects eligible for Part 2 each have nanoscale particle antimicrobial composition applied to 6 sites across the upper chest or abdomen. Swab cultures are obtained at specified time points over a period of 7 days. In addition, subjects in Part 2 have 2 peripheral catheters inserted, one in each arm. One catheter insertion site will be treated with nanoscale particle antimicrobial composition (following treatment with isopropyl alcohol) and the other site will be treated with chlorhexidine 2%/isopropyl alcohol. Swab cultures are obtained at specified time points over a period of 7 days.

Primary Outcome Measurements: change in mean number of skin bacterial counts from baseline to 73 hours; change in mean number of skin bacterial counts from baseline to 7 days, number of subjects with significantly colonized catheters, defined as greater than or equal to 15 colony forming units (CFUs) at 0 hours.

1.-112. (canceled)

113. A composition comprising: (a) one or more germinants; (b) polyolprepolymer-2 or poly(styrene-co-maleic anhydride) copolymers (SMA); (c) one or more nanoscale particle having a particle size of about 5 nm to about 200 nm; and (d) a carrier.

114. The composition of claim 113, wherein at least one nanoscale particle comprises an ionic silver salt, elemental silver, or combinations thereof.

115. The composition of claim 114, wherein the elemental silver is colloidal silver.

116. The composition of claim 113, wherein at least one nanoscale particle comprises titanium dioxide.

117. The composition of claim 113, wherein the nanoscale particle has a particle size of about 100 to about 200 nm.

118. The composition of claim 113, wherein the germinant is:
(a) a compound of Formula I: 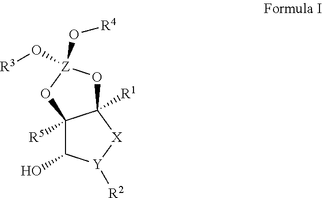
wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl; X is O, CH₂, or S;
Y is CH or N; and
Z is C or B;
or salts thereof;
(b) a compound of Formula II: 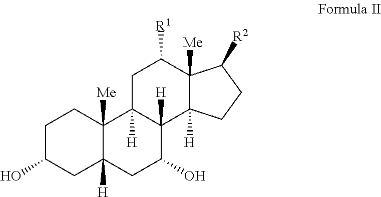
wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
wherein each R⁶ is independently H or alkyl; or
has the structure of Formula IIa: 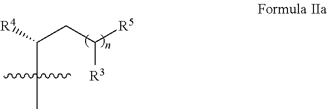
wherein
each R³ is independently H, alkyl, alkoxy, CO₂R⁶, or halo;
R⁴ is H, alkyl, OH, alkoxy, or halo;
R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
each R⁶ is independently H or alkyl; and
n is 0, 1, 2, 3, 4, or 5; or
has the structure of Formula IIb: 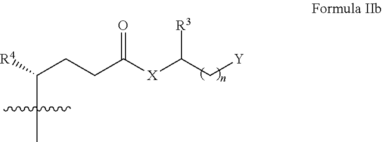
wherein
R³ is H, alkyl, alkoxy, CO₂R⁶, or halo;
R⁴ is H, alkyl, OH, alkoxy, or halo;
X is NR⁶, O or C(R⁶)₂;
Y is SO₃H, PO₃H, CO₂R⁶, CONR⁶, H, alkyl, OH, alkoxy, halo, aminoalkyl, or amino; and
each R⁶ is independently H or alkyl;
n is 0, 1, 2, 3, 4, or 5; or
a salt thereof.

119. The composition of claim 113, wherein the germinant is selected from the group consisting of:
2,3,3,4(2H)-Furantetrol, dihydro-2-methyl-, (2R,4S)-
2,3,3,4(2/-I)-Furantetrol, dihydro-2-methyl-, (2S,4S)-
2,3,3,4(2H)-Furantetrol, dihydro-2-methyl-, (45)-
2,3,3,4(2H)-Furantetrol, dihydro-2-(methyl-13C)-, (2S,4S)- (9CI)
2,3,3,4(2H)-Furantetrol, dihydro-2-(methyl-13C)-, (2R,4S)- (9CI)
2,3,3,4(2H)-Furantetrol, dihydro-2-(methyl-13C)-, (4S)-
2,3,3,4(2H)-Furantetrol-2-13C, dihydro-2-(methyl-13C)-, (4S)-
2,3,3(2H)-Furantriol, dihydro-4-methoxy-2-methyl-
2,3,3(2H)-Furantriol, 4-ethoxydihydro-2-methyl
2,3,3(2H)-Furantriol, dihydro-2-methyl-4-propoxy-
3-Furanol, tetrahydro-2,4,4,5-tetramethoxy-5-methyl-
2,3,3,4(2H)-Furantetrol, dihydro-2-(trifluoromethyl)-,(4S)-
2,3,3(2H)-Furantriol, 4-(hexyloxy)dihydro-2-methyl-
2,3,3(2H)-Furantriol, dihydro-2-methyl-4-(phenylmethoxy)-
Borate(1-),[[(2S,3S,4S)-dihydro-2-methyl-2,3,3,4(2H)-furantetrolato(2-)-KO3,KO4]dihydroxy-, (T-4)-
3,4-Furandiol, tetrahydro-2,5-dimethoxy-2-methyl- (7CI)
erythro-2-Hexulofuranose, 1,6-dideoxy-5-C-niethoxy-(5E1)-(9CI)
2-Hexulofuranoside, methyl 1,6-dideoxy-5-C-methoxy-, (9CI)
α-D-Psicofuranose, 5-C-hydroxy- (9CI)
β-D-Psicofuranose, 5-C-hydroxy- (9CI)
β-L-Tagatofuranose, 5-C-hydroxy- (9CI)
3,4-Furandiol, tetrahydro-2-(hydroxymethyl)-2,5-dimethoxy- (9CI)
3-Furanol, tetrahydro-2,4,4,5-tetramethoxy-5-methyl-
2-Furoic acid, tetrahydro-3,4-dihydroxy-2,5-dimethoxy-(6CI)
2-Furaldehyde, tetrahydro-3,4-dihydroxy-2,5-dimethoxy-, dimethyl acetal (6CI)
3,4-Furandiol, tetrahydro-2,5-dimethoxy-2-methyl-, diacetate (7CI)
2-Furoic acid, tetrahydro-3,4-dihydroxy-2,5-dimethoxy-, methyl ester (6C1,7CI)
D-erythro-Pentofuranose, 5-deoxy-4-C-hydroxy-2,3-O-(1-methylethylidene)-, (4ξ)-(9CI)
β-D-Fructofuranose, 5-C-hydroxy-, 1-(dihydrogen phosphate) (9CI)
2-Furoic acid, tetrahydro-3,4-dihydroxy-2,5-dimethoxy-5-methyl-, methyl ester (7CI)
2-Furamide, tetrahydro-3,4-dihydroxy-2,5-dimethoxy- (6CI)
Furfuryl alcohol, tetrahydro-3,4-dihydroxy-2,5-dimethoxy-, acetate (6CI)
α-D-Glucopyranoside, 4-C-hydroxy-α-D-arabinofuranosyl (9CI)
Pentofuranoside, methyl 5-deoxy-3-C-(dimethoxymethyl)-4-C-methoxy-(9CI)
D-erythro-Pentofuranoside, methyl 5-deoxy-4-C-hydroxy-2,3-O-(1-methylethylidene)-, (4ξ)- (9CI)
D-erythro-Pentofuranoside, methyl 5-deoxy-4-C-methoxy-2,3-O-(1-methylethylidene)-, (4ξ)- (9CI)
α-D-Fructofuranose, 5-C-hydroxy-, 1,6-bis(dihydrogen phosphate) (9CI)
β-D-Fructofuranose, 5-C-hydroxy-, 1,6-bis(dihydrogen phosphate) (9CI)
α-D-ribo-Hexopyranosid-3-ulose, (4R)-4-C-hydroxy-β-D-arabinofuranosyl (9CI)
α-D-Galactopyranoside, (5S)-5-C-hydroxy-β-D-threo-2-pentulofuranosyl (9CI)
α-D-Glucopyranoside, (5 S)-5-C-hydroxy-β-D-threo-2-pentulofuranosyl (9CI)
2,7,12,13-Tetraoxatricyclo[7.2.1.1^{3,6}]tridecane-4,5,9,10,11-pentol, 3-(hydroxymethyl)-1,6-dimethyl-, (4S,5R,10S,11R)-
β-D-Xylofuranose, 1,5-anhydro-4-C-(c-D-glucopyranosyloxy)- (9CI)
2-Furoic acid, tetrahydro-3,4-dihydroxy-2,5-dimethoxy-, -methyl ester, diacetate (7CI)
β-D-threo-2,5-Hexodiulo-2,6-pyranose, 5-hydrate, 5,2¹:5,3¹-dianhydride with 5-C-hydroxy-α-L-sorbofuranose (9CI)
D-erythro-Pentofuranose, 5-bromo-5-deoxy-4-C-hydroxy-2,3-O-(1-methylethylidene)-, (4ξ)-
D-erythro-Pentofuranose, 5-deoxy-4-C-hydroxy-5-iodo-2,3-O-(1-methylethylidene)-, (4ξ)-
3,4-Furandiol, 2-[(benzoyloxy)methyl]tetrahydro-2,5-dimethoxy-(9CI)
β-D-ribo-Pentofuranoside, methyl 5-bromo-5-deoxy-4-C-hydroxy-2,3,O-(1-methylethylidene)-
α-L-lyxo-Pentofuranoside, methyl 5-bromo-5-deoxy-4-C-hydroxy-2,3-O-(1-methylethylidene)-
α-D-ribo-Pentofuranoside, methyl 5-bromo-5-deoxy-4-C-hydroxy-2,3-O-(1-methylethylidene)-
β-L-lyxo-Pentofuranoside, methyl 5-bromo-5-deoxy-4-C-hydroxy-2,3-O-(1-methylethylidene)-
D-erythro-Pentofuranoside, methyl 5-bromo-5-deoxy-4-C-hydroxy-2,3-O-(1-methylethylidene)-, (4ξ)-
D-erythro-Pentofuranoside, methyl 5-deoxy-4-C-hydroxy-5-iodo-2,3-O-(1-methylethylidene)-, (4ξ)-
Pentofuranoside, methyl 5-deoxy-3-C-(dimethoxymethyl)-4-C-methoxy-2,3-bis-O-(trimethylsilyl)- (9CI)
Furo[3,4-d]-1,3,2-dioxaphosphol-4-ol, tetrahydro-2,6-dimethoxy-4,6-dimethyl-, 2-oxide (9CI)
D-erythro-L-ribo-5-Nonuto-5,2-furanose, 5,9-anhydro-1,6,7,8-tetradeoxy-3-O-[(1,1-dimethylethyl)dimethylsilyl]2-C-hydroxy-6,8-dimethyl-4-O-methyl-, (5S)- (9CI)
Pentofuranoside, methyl 5-deoxy-3-C-(dimethoxymethyl)-4-C-methoxy-, diacetate (9CI)
13,14-Dioxatricyclo[8.2.1.1^{4,7}]tetradecane-5,6,11,12-tetrol, 1,4,7,10-tetramethoxy-
3,4-Furandiol, tetrahydro-2,5-dimethoxy-2-methyl-, dibenzoate (7CI)
β-D-Ribofuranoside, (2-nitrophenyl)methyl 4-C-methoxy-
β-L-erythro-Hexofuranosid-5-ulose, methyl 6-deoxy-4-C-methoxy-2,3-O-(1-methylethylidene)-, (S)- (9CI)
α-D-Lyxofuranose, 4-C-ethoxy-5-O-(phenylmethyl)-, triacetate (9CI)
β-D-Lyxofuranose, 4-C-ethoxy-5-O-(phenylmethyl)-, triacetate (9CI)
β-D-Ribofuranoside, (2-nitrophenyl)methyl 4-C-methoxy-2-O-methyl-
α-D-Glucofuranose, 4-0-methyl-1,2:5,6-bis-O-(1-methylethylidene)-2,3,7-Trioxabicyclo[2.2.1]heptane-5,6-diol, 1,4-dimethyl-, dinitrate, (5-endo, 6-exo)- (9CI)
Galactitol, 2,5-anhydro-1,6-dideoxy-2,5-dimethoxy-3,4-di-C-methyl-, cyclic 3,4-(hydrogen phosphate) (8CI)
a-D-Tagatofuranoside, methyl 1,6-dideoxy-5-C-methoxy-3,4-di-C-methyl-, cyclic hydrogen phosphate (9CI)
β-D-Riboruranoside, methyl 4-C-(1-cyanoethoxy)-5-deoxy-2,3-O-(1-methylethylidene)- (9CI)
Phosphoric acid, methyl ester, cyclic 3,4-ester with tetrahydro-5-methoxy-2,3,4,5-tetramethyl-2,3,4-furantriol (7CI)
α-L-Tagatofuranose, 2,5-anhydro-1,6-dideoxy-5-C-methoxy-3,4-di-C-methyl-, cyclic 3,4-(methyl phosphate), (R)-(9CI)
α-L-Tagatofuranose, 1,6-dideoxy-5-C-methoxy-3,4-di-C-methyl-, cyclic 3,4-(methyl phosphate), (S)- (9CI)
Fructofuranose, O-α-D-galactopyranosyl-(1→6)-O-α-D-glucopyranosyl-6-t-(1→4)-, β-D- (8CI)
Raffinose-6′-t (8CI)
SaH-Oxireno[8,8a]naphtho[2,3-b]furan-5a,6-diol, decahydro-7,8a-dimethoxy-4,4a,6-trimethyl-, (1aR,4S,4aR,5aR,6S,7S,8aS,9aS)-
Molybdate(1-), [μ-[1,6-dideoxy-5-C-hydroxy-3,4-di-C-methyl-α-psicofuranosato(3-)-O²,O³:O³,O⁴]]-μ-oxotetraoxodi-, steroisomer (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-) (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-), compd. with 1,1′-oxybis[ethane](2:1) (9CI)
9a H-4a,8-Epoxy-1,3-dioxolo[4,5]furo[2,3-d]oxepin-9a-ol, hexahydro-2,2,8-trimethyl-, [3aS-(3aα,4aα,8α,9aβ,9bα)]-(9CI)
α-Lyxofuranoside, methyl 3-C-[(benzoyloxy)methyl]-5-deoxy-4-C-methoxy-, 2-acetate (9CI)
2-Furoic acid, tetrahydro-3,4-dihydroxy-2,5-dimethoxy-, methyl ester, dibenzoate (7CI)
β-D-ribo-Heptofuranoside, methyl 4,6-anhydro-5,7-dideoxy-4-C-hydroxy-6-Cmethyl-2,3-O-(1-methylethylidene)- (9CI)
β-L-Sorbofuranose, 5-C-hydroxy-1,3:4,6-bis-O-(phenylmethylene)-, [1(R),4(R)]- (9CI)
β-L-Sorbofuranose, 5-C-hydroxy-1,3:4,6-bis-O-(phenylmethylene)-(9CI)
α-D-Glucofuranose, 4-O-methyl-1,2:5,6-bis-O-(1-methylethylidene)-, 3-acetate
α-D-Galactofuranose, 4-O-methyl-1,2:5,6-bis-O-(1-methylethylidene)-, 3-acetate
2,4,6-Metheno-2H-cyclopenta[g]furo[2,3,4-ij][2]benzopyran-2,5a,6a,9a,9b,9c(2aH,6H,7H)-hexol, tetrahydro-2a,6,9-trimethyl-4-(1-methylethyl)-, (2S,2aS,4S,5aR,6S,6aS,9R,9aS,9bR,9cS,10S)- (9CI)
Furo[2,3-d:4,5-d]bis[1,3]dioxole, tetrahydro-2,2,3a,6,6,7b-hexamethyl-, (3a,4aα,7aβ,7bβ)- (9CI)
1,4a-(Epoxymethano)-4aH-xanthen-9 (2H)-one, 1,3,4,9a-tetrahydro-1,9a-dihydroxy-11-methoxy-
α-L-Sorbofuranose, 5-C-methoxy-1,3:4,6-bis-O-(phenylmethylene)-, [1(R),4(R),5S]- (9CI)
Furo[2,3-d:4,5-d]bis[1,3]dioxole, tetrahydro-2,2,3a,4a,6,6-hexamethyl-, (3aα,4aβ,7aβ,7bα)- (9CI)
Spiro[furan-2(3H),4′(3′aH)-furo[3,4-d][1,3]dioxole], 6′,6′a-dihydro-6′-methoxy-2′,2′-dimethyl-, (2R,3′aS,6′R,6′aR)-
Octofuranosiduronic acid, methyl 3,6-anhydro-5-deoxy-4-C-methoxy-6-C-(methoxycarbonyl)-, methyl ester (9CI)
β-L-erythro-Hexofuranosid-5-ulose, methyl 6-deoxy-4-C-methoxy-2,3-O-(1-methylethylidene)-, oxime, (4R)- (9CI)
2-Propanone, 1-hydroxy-1-[tetrahydro-6-hydroxy-2,3a,5-trimethyl-5,2-(epoxymethano)furo[2,3-d]-1,3-dioxol-8-yl]-, [2R-[2α,3aβ,5α,6β,6aβ,8S*(R*)]]- (9CI)
β-L-erythro-β-L-lyxo-Decofuranos-7-ulo-7,10-furanose, 10-C-(acetyloxy)-3,7-anhydro-5,6-dideoxy-, tetraacetate, (10R)- (9CI)
Inulobiose, octaacetate (5CI)
2,3,7-Trioxabicyclo[2.2.1]heptane-1-acetonitrile, 5-[[(1,1-dimethylethyl)dimethylsilyl]oxy]-6-methoxy-4-methyl-, (1R,4S,5R,6R)-
D-gluco-Nonitol, 2,5-anhydro-1,6,7,8-tetradeoxy-3-O-[(1,1-dimethylethyl)dimethylsilyl]-2,5-C-epidioxy-6,8-dimethyl-4-O-methyl-, (2ξ,5ξ)- (9CI)
β-D-Lyxofuranose, 4-C-ethoxy-2,3-O-(1-methylethylidene)-5-O-(phenylmethyl)-, acetate (9CI)
2-Heptenal, 6-[(5S,6R)-5-hydroxy-6-methoxy-4-methyl-2,3,7-trioxabicyclo[2.2.1]hept-1-yl]-4-methyl-, (2E,4S,6R)-
Spiro[1,2-dioxin-3(6H),5′(3′aH)-furo[2,3-d][1,3]dioxole]-6,6′-diol, 6′,6′a-dihydro-2′,2′,6-trimethyl-, 6′-acetate, (3R,3′aS,6′R,6′aR)-
Spiro[1,2-dioxin-3(6H), 5′(3′aH)-furo[2,3-d][1,3]dioxole]-6,6′-diol, 6′,6′a-dihydro-2′,2′,6-trimethyl-, 6′-acetate, (3S,3′aS,6′R,6′aR)-
5,8-Epoxy-1,4-dioxino[2,3-d][1,2]dioxin, hexahydro-4a,5,8,8a-tetramethyl-, (4aR,5S,8R,8aS)-rel-
5,8-Epoxy-1,4-dioxino[2,3-d][1,2]dioxin, hexahydro-4a,5,8,8a-tetramethyl-, (4aR,5R,8S,8aS)-rel-
5,8-Epoxy-1,4-dioxino[2,3-d][1,2]dioxin, hexahydro-4a,5,8,8a-tetramethyl-
4H-1-Benzopyran-4-one, 2-(3,4-dihydroxyphenyl)-5,6-dihydroxy-7-[(4-C-hydroxy-α-D-ribofuranosyl)oxy]- (9CI)
Furo[2,3-d:4,5-d]bis[1,3]dioxole, tetrahydro-2,2,3a,4a,6,6,7a,7b-octamethyl-, (3aα,4aα,17aβ,7bβ)- (9CI)
6,9-Epoxy-2H-o-dioxino[4,5-b][1,4]dioxepin, hexahydro-5a,6,9,9a-tetramethyl-, stereoisomer (8CI)
6,9-Epoxy-2H-o-dioxino[4,5-b][1,4]dioxepin, hexahydro-5a,6,9,9a-tetramethyl-, stereoisomer (8CI)
L-gulo-Nonose, 5,8-anhydro-2,3,4,9-tetradeoxy-7-O-[(1,1-dimethylethyl)dimethylsilyl]-5,8-C-epidioxy-2,4-dimethyl-6-O-methyl-, (5ξ,8ξ)- (9CI)
α-L-Sorbofuranose, 5-C-methoxy-1,3:4,6-bis-O-(phenylmethylene)-, acetate, [1(R),4(R),5S]- (9CI)
Spiro[1,2-dioxin-3(6H),5′(3′aH)-furo[2,3-d)[1,3]dioxol]-6-ol, 6′,6′a-dihydro-6′-methoxy-6-methyl-2′-(trichloromethyl)-, (2′S,3S,3′aS,6′R,6′aR)-
Spiro[1,2-dioxin-3(6H),5′(3′aH)-furo[2,3-d)[1,3]dioxol]-6-ol, 6′,6′a-dihydro-6′-methoxy-6-methyl-2′-(trichloromethyl)-, (2′R,3R,3′aS,6′R,6′aR)-
7,10-Epoxy[1,2]dioxino[4,5-b][1,4]dioxocin, octahydro-6a,7,10,10a-tetramethyl-
Furo[2,3-d:4,5-d]bis[1,3]dioxole-3a(4aH)-carboxylic acid, dihydro-2,2,6,6-tetramethyl- (9CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, (3aR,3′aS,3bR,5S,6′S,6′aR,7aS,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, (3aR,3′aS,3bR,5S,6′S,6′aR,7aR,8aR)- (9CI)
Spiro[1,2-dioxin-3 (6H),5′(3′aH)-furo[2,3-d][1,3]dioxole]-6,6′-diol, 6′,6′a-dihydro-2′,2′,6-trimethyl-, 6,6′-diacetate, (3S,3′aS,6′R,6′aR)-
Spiro[1,2-dioxin-3 (6H),5′(3′aH)-furo[2,3-d][1,3]dioxole]-6,6′-diol, 6′,6′a-dihydro-2′,2′,6-trimethyl-, 6,6′-diacetate, (3R,3′aS,6′R,6′aR)-
2H-Naphtho[1,2-b]pyran-2-one, 6-[[5-C-(α-D-glucopyranosyloxy)-β-D-fructofuranosyl]oxy]-7-hydroxy-3-methyl-
Spiro[furo[3,4-d]-1,3-dioxole-4(3aH),5′(4′H)-isoxazole], 6,6a-dihydro-6-methoxy-2,2,3′-trimethyl-, (3aS,4R,6R,6aR)-
Spiro[1,2-dioxin-3 (6H),5′(3′aH)-furo[2,3-d][1,3]dioxole]-6,6′-diol, 6′,6′a-dihydro-6-methyl-2′-(trichloromethyl)-, 6′-acetate, (2′S,3S,3′aS,6′R,6′aR)-
Spiro[1,2-dioxin-3(6H), 5′(3′aH)-furo[2,3-d][1,3]dioxole]-6,6′-diol, 6′,6′a-dihydro-6-methyl-2′-(trichloromethyl)-, 6′-acetate, (2′R,3R,3′aS,6′R,6′aR)-
α-D-Glucofuranose, 4-C-hydroxy-1,2:5,6-bis-O-(1-methylethylidene)-, 3-(3-chlorobenzoate)
Spiro[5H-1,3-dioxolo[4,5]furo[3,2-b]pyran-5,5′(3′aH)-furo[2,3-d][1,3]dioxol]-6′-ol, 3a,6,6′,6′a,7,8a-hexahydro-2,2,2′,2′-tetramethyl-, (3aR,3′aS,5S,6′S,6′aR,8aR)-
Spiro[furo[3,4-d]-1,3-dioxole-4(3aH),5′(4′H)-isoxazole], 3′-ethyl-6,6a-dihydro-6-methoxy-2,2-dimethyl-, (3aS,4R,6R,6aR)-
1,4-Epoxy-o-dioxino[4,5-b][1,4]benzodioxin, 1,4,4a, 10a-tetrahydro-1,4,4a, 10a-tetramethyl-, stereoisomer (8CI)
1,4-Epoxy-o-dioxino[4,5-b][1,4]benzodioxin, 1,4,4a, 10a-tetrahydro-1,4,4a, 10a-tetramethyl-, stereoisomer (8CI)
1,4:6,9-Diepoxy-p-dioxino[2,3-d:5,6-d]bis-o-dioxin, octahydro-1,4,4a,5a,6,9,9a, 10a-octamethyl-, stereoisomer (8CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2′,2′-tetramethyl-, (3aR,3′aS,3bR,5S,6′S,6′aR,7aS,8aR)- (9CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2′,2′-tetramethyl-, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, 6′-acetate, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, 6′-(1-aminoethyl)-3a,3b,6,6′,6′a,7,7a,8a-octahydro-2,2,2′,2′-tetramethyl-, (3aR,3′aS,3bS,5S,6′S,6′aR,7aR,8aR)-
L-glycero-β-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-β-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
β-L-threo-Pentofuranose, 4-C-(acetyloxy)-5-deoxy-5-fluoro-1,2-O-(1-methylethylidene)-, 4-methylbenzenesulfonate, (4ξ)- (9CI)
α-D-Glucopyranoside, methyl 6-deoxy-4-O-(4-C-hydroxy-2,3,5-tri-O-methyl-α-D-arabinofuranosyl)-2,3-di-O-methyl-6-[[(4-nitrophenyl) sulfonyl]amino]-
Spiro[furo[3,4-d]-1,3-dioxole-4(3aH),5′(4′H)-isoxazole], 6,6a-dihydro-6-methoxy-2,2-dimethyl-3′-[(3aR,4R,6R,6aR)-tetrahydro-6-methoxy-2,2-dimethylfuro[3,4-d]-1,3-dioxol-4-yl]-, (3aS,4R,6R,6aR)
Spiro[furo[3,4-d]-1,3-dioxole-4(3aH),2′(3′H)-pyrrolo[1,2-b]isoxazole], hexahydro-6-methoxy-4′,5′-bis(methoxymethoxy)-2,2-dimethyl-, (2′R,3aS,3′aS,4′S,5′S,6R,6aR)-
5,2,9-Ethanylylidene-1-benzoxepin-8,11(2H)-dione, 4-[(2R,3R)-3,4-dihydro-3,5,7-trihydroxy-2H-1-benzopyran-2-yl]-6-[(2S,3S)-3,4-dihydro-3,5,7-trihydroxy-2H-1-benzopyran-2-yl]-5,5a,9,9a-tetrahydro-2,9,9a,10-tetrahydroxy-, (2R,5S,5aS,9S,9aS,10R)- (9CI)
Spiro[5H-1,3-dioxolo[4,5]furo[3,2-b]pyran-5,5′(3′a H)-furo[2,3-d][1,3]dioxol]-6′-ol, 3a,6,6′,6′a,7,8a-hexahydro-2,2,2′,2′-tetramethyl-, 6′-acetate, (3aR,3′aS,5S,6′S,6′aR,8aR)-
Spiro[furo[3,4-d)-1,3-dioxole-4(3aH),5′(4′H)-isoxazole], 6,6a-dihydro-6-methoxy-2,2-dimethyl-3′-phenyl-, (3aS,4R,6R,6aR)-
1,4-Epoxycyclobuta[5,6]-p-dioxino[2,3-d)-o-dioxin, 1,4,4a,5a,7a,8a-hexahydro-1,4,4a,5a,6,7,7a,8a-octamethyl-, stereoisomer (8CI)
1,4-Epoxycyclobuta[5,6]-p-dioxino[2,3-d)-o-dioxin, 1,4,4a,5a,7a,8a-hexahydro-1,4,4a,5a,6,7,7a,8a-octamethyl-, stereoisomer (8CI)
Spiro[furo[3,4-d)-1,3-dioxole4(3aH),6′-[6H]1,2]oxazine]-3′-carboxylic acid, hexahydro-6-methoxy-2,2-dimethyl-, ethyl ester, (3′ R,3a5,4R,6R,6aR)-
Spiro[furo[3,4-d)-1,3-dioxole-4(3aH),5′-isoxazolidine], dihydro-6-methoxy-2,2-dimethyl-2′-(phenylmethyl)-, (3aS,4R,6R,6aR)-
2H-Naphtho[1,2-b]pyran-2-one, 7-(acetyloxy)-3-methyl-6-[[1,3,4,6-tetra-O-acetyl-5-C-[(2,3,4,6-tetra-O-acetyl-α-D-glucopyranosyl)oxy]-β-D-fructofuranosyl]oxy]-
Spiro[furo[3,4-d)-1,3-dioxole-4(3aH),5′(4′H)-isoxazole), 6,6a-dihydro-6-methoxy-2,2-dimethyl-3′-(4-methylphenyl)-, (3aS,4R,6R,6aR)-
3,6-Epoxy-2H,8H-pyrimido[6,1-b][1,3]-oxazocine-8,10(9H)-(dione, 3,4,5,6-tetrahydro-4-hydroxy-, acetate (ester)(7CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, 6′-benzoate, (3aR,3′aS,3bS,5S,6′S,6′aR,7aR,8aR)-
Spiro[5H-1,3-dioxolo[4,5][furo[3,2-b]pyran-5,5′(3′aH)-furo[2,3-d)[1,3]dioxol]-6′-ol, octahydro-2,2,2′,2′-tetramethyl-6′-(nitromethyl)-, (3aR,3′aS,3bS,5S,6′S,6′aR,7aR,8aR)-
Spiro[furo[3,4-d)-1,3-dioxole-4(3aH),6′-[6H]1,2]oxazine]-3′-carboxylic acid, 4′,5′,6,6a-tetrahydro-6-methoxy-2,2-dimethyl-, ethyl ester, (3aS,4R,6R,6aR)-
Hexanoic acid, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Acetamide, N-[1-[(2′R,3′R,3aS,3′aS,5′S,6S,6aR,7′aR)-2′,3′-bis(acetyloxy)-2′,3′,3′a,6,6′,6a,7′,7′a-octahydro-6-hydroxy-2,2-dimethylspiro[furo[2,3-d]-1,3-dioxole-5(3aH),5′-[5H]furo[3,2-b]pyran]-6-yl]ethyl]-
Spiro[5H-1,3-dioxolo[4,5]furo[3,2-b]pyran-5,5′(3′aH)-furo[2,3-d)[1,3]dioxol]-6′-ol, 3a,6,6′,6′a,7,8a-hexahydro-2,2,2′,2′-tetramethyl-6′-(nitromethyl)-, (3aR,3′aS,5S,6′S,6′aR,8aR)-
Spiro[5H-1,3-dioxolo[4,5]furo[3,2-b]pyran-5,5′(3′aH)-furo[2,3-d)[1,3]dioxol]-6′-ol, 3a,6,6′,6′a,7,8a-hexahydro-2,2,2′,2′-tetramethyl-, 6′-benzoate, (3aR,3′aS,5S,6′S,6′aR,8aR)-
Spiro[furo[3,4-d)-1,3-dioxole-4(3aH),5′(4′H)-isoxazole], 3′-(2,6-dichlorophenyl)-6,6a-dihydro-6-methoxy-2,2-dimethyl-, (3aS,4R,6R,6aR)-
1,6,11,14,16,20,23,24-Octaoxahexaspiro[3.0.3.0.0.4.0.3.0.3.1.1]tetracosane
Hexanoic acid, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3)dioxol]-6′-yl ester
Benzoic acid, 4-chloro-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Benzoic acid, 4-bromo-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Spiro[5H-1,3-dioxolo[4,5]furo[3,2-b]pyran-5,5′(3′aH)-furo[2,3-d][1,3]dioxol]-6′-ol, 3a,6,6′,6′a,7,8a-hexahydro-2,2,2′,2′-tetramethyl-6′-(1-nitroethyl)-, (3′aS,3aR,5S,6′S,6′aR,8aR)-
Sorbofuranose, 2,3:4,6-di-O-isopropylidene-, p-toluenesulfonate, α-L-(7CI)
Benzoic acid, 4-chloro-, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Benzoic acid, 4-bromo-, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Spiro[furo[2,3-d]-1,3-dioxole-5(3aH),6′-[6H]1,2]oxazine]-3′-carboxylic acid, hexahydro-2,2-dimethyl-6-(phenylmethoxy)-, ethyl ester, (3′R,3aS,5R,6R,6aR)-
Spiro[furo[3,4-d]-1,3-dioxole-4(3aH),6′-[6H-1,2]oxazine]-2′,3′(3′H)-dicarboxylic acid, tetrahydro-6-methoxy-2,2-dimethyl-, 2′-(1,1-dimethylethyl) 3′-ethyl ester, (3′R,3aS,4R,6R,6aR)-
Spiro[furo[2,3-d]-1,3-dioxole-5(3aH),6′-[6H]1,2]oxazine]-3′-carboxylic acid, 4′,5′,6,6a-tetrahydro-2,2-dimethyl-6-(phenylmethoxy)-, ethyl ester, (3aS,5R,6R,6aR)-
β-D-Galactofuranose, 3,4-O-[(acetylamino)phenylmethylene]-4-C-hydroxy-, 1-acetate 2,5,6-tribenzoate (9CI)
β-D-Galactofuranose, 3,4-O-[(2,5-dioxo-1-pyrrolidinyl)phenylmethylene]-4-C-hydroxy-, 1-acetate 2,5,6-tribenzoate (9CI)
Osmium, tetraoxotetrakis(pyridine)[μ-[1-(tetrahydro-2,3,4,5-tetrahydroxy-2-furanyl) ethanonato(4-)]]di-, stereoisomer (9CI)
β-D-Galactofuranose, 3,4-O-[(1,3-dihydro-1,3-dioxo-2H-isoindol-2-yl)phenylmethylene]-4-C-hydroxy-, 1-acetate 2,5,6-tribenzoate (9CI)
Piperazinone, 4-[(dihydro-2,2,6,6-tetramethylfuro[2,3-d;4,5-d]bis[1,3]dioxol-3a(4aH)-yl)carbonyl]-6-ethyl-1-[(4-methylphenyl)methyl]- (9CI)
Quinoline, 5,7-dichloro-1-[(dihydro-2,2,6,6-tetramethylfuro[2,3-d:4,5-d]bis[1,3]dioxol-3a(4aH)-yl) carbonyl]-4-(4-fluorophenoxy)-1,2-dihydro-(9CI);
and combinations thereof.

120. The composition of claim 113, wherein the carrier is ethyl alcohol.

121. The composition of claim 113, further comprising a coloring agent that adheres to the skin during use to indicate compliant application of product.

122. The composition of claim 113, further comprising titanium oxide USP #3228.

123. A composition comprising: (a) one or more germinants; (b) polyolprepolymer-2 or poly(styrene-co-maleic anhydride) copolymers (SMA); and (d) a carrier; wherein the germinant is:
(a) a compound of Formula I: 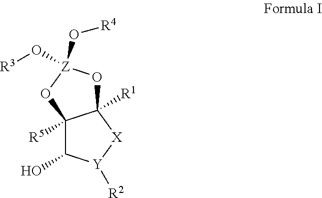
wherein
R¹ is H, alkyl, cycloalkyl, OH, alkoxy, or halo;
R² is H, alkyl, cycloalkyl, OH, or alkoxy;
R³ and R⁴ are independently alkyl or H;
R⁵ is alkyl or cycloalkyl; X is O, CH₂, or S;
Y is CH or N; and
Z is C or B;
or salts thereof; or
(b) a compound of Formula II: 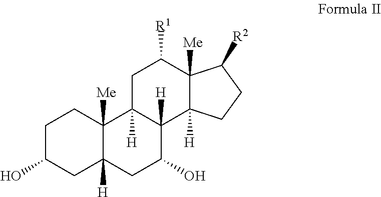
wherein
R¹ is H, alkyl, OH, alkoxy, or halo;
R² is OH, alkyl, alkoxy, halo, amino, aminoalkyl, aminodialkyl, alkylamino, alkylaminoalkyl, CO₂R⁶, COR⁶, or CON(R⁶)₂;
wherein each R⁶ is independently H or alkyl; or
has the structure of Formula IIa: 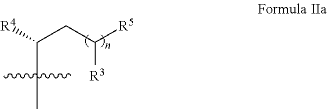
wherein
each R³ is independently H, alkyl, alkoxy, CO₂R⁶, or halo;
R⁴ is H, alkyl, OH, alkoxy, or halo;
R⁵ is H, alkyl, OH, alkoxy, halo, SO₃H, PO₃H, CO₂R⁶, COR⁶, CONR⁶, aminoalkyl, amino, N(R⁶)benzyl, or N(R⁶)phenyl;
each R⁶ is independently H or alkyl; and
n is 0, 1, 2, 3, 4, or 5; or
has the structure of Formula IIb: 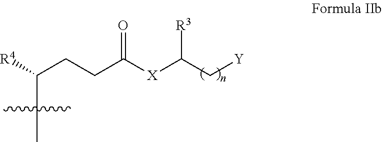
wherein
R³ is H, alkyl, alkoxy, CO₂R⁶, or halo;
R⁴ is H, alkyl, OH, alkoxy, or halo;
X is NR⁶, O or C(R⁶)₂;
Y is SO₃H, PO₃H, CO₂R⁶, CONR⁶, H, alkyl, OH, alkoxy, halo, aminoalkyl, or amino; and
each R⁶ is independently H or alkyl;
n is 0, 1, 2, 3, 4, or 5; or
a salt thereof; or
2,3,3,4(2H)-Furantetrol, dihydro-2-methyl-, (2R,4S)-
2,3,3,4(2/-I)-Furantetrol, dihydro-2-methyl-, (2S,4S)-
2,3,3,4(2H)-Furantetrol, dihydro-2-methyl-, (45)-
2,3,3,4(2H)-Furantetrol, dihydro-2-(methyl-13C)-, (2S,4S)- (9CI)
2,3,3,4(2H)-Furantetrol, dihydro-2-(methyl-13C)-, (2R,4S)- (9CI)
2,3,3,4(2H)-Furantetrol, dihydro-2-(methyl-13C)-, (4S)-
2,3,3,4(2H)-Furantetrol-2-13C, dihydro-2-(methyl-13C)-, (4S)-
2,3,3(2H)-Furantriol, dihydro-4-methoxy-2-methyl-
2,3,3(2H)-Furantriol, 4-ethoxydihydro-2-methyl
2,3,3(2H)-Furantriol, dihydro-2-methyl-4-propoxy-
3-Furanol, tetrahydro-2,4,4,5-tetramethoxy-5-methyl-
2,3,3,4(2H)-Furantetrol, dihydro-2-(trifluoromethyl)-,(4S)-
2,3,3(2H)-Furantriol, 4-(hexyloxy)dihydro-2-methyl-
2,3,3(2H)-Furantriol, dihydro-2-methyl-4-(phenylmethoxy)-
Borate(1-),[[(2S,3S,4S)-dihydro-2-methyl-2,3,3,4(2H)-furantetrolato(2-)-KO3,KO4]dihydroxy-, (T-4)-
3,4-Furandiol, tetrahydro-2,5-dimethoxy-2-methyl- (7CI)
erythro-2-Hexulofuranose, 1,6-dideoxy-5-C-niethoxy-(5E1)-(9CI)
2-Hexulofuranoside, methyl 1,6-dideoxy-5-C-methoxy-, (9CI)
α-D-Psicofuranose, 5-C-hydroxy- (9CI)
β-D-Psicofuranose, 5-C-hydroxy- (9CI)
β-L-Tagatofuranose, 5-C-hydroxy- (9CI)
3,4-Furandiol, tetrahydro-2-(hydroxymethyl)-2,5-dimethoxy- (9CI)
3-Furanol, tetrahydro-2,4,4,5-tetramethoxy-5-methyl-
2-Furoic acid, tetrahydro-3,4-dihydroxy-2,5-dimethoxy-(6CI)
2-Furaldehyde, tetrahydro-3,4-dihydroxy-2,5-dimethoxy-, dimethyl acetal (6CI)
3,4-Furandiol, tetrahydro-2,5-dimethoxy-2-methyl-, diacetate (7CI)
2-Furoic acid, tetrahydro-3,4-dihydroxy-2,5-dimethoxy-, methyl ester (6C1,7CI)
D-erythro-Pentofuranose, 5-deoxy-4-C-hydroxy-2,3-O-(1-methylethylidene)-, (4ξ)-(9CI)
β-D-Fructofuranose, 5-C-hydroxy-, 1-(dihydrogen phosphate) (9CI)
2-Furoic acid, tetrahydro-3,4-dihydroxy-2,5-dimethoxy-5-methyl-, methyl ester (7CI)
2-Furamide, tetrahydro-3,4-dihydroxy-2,5-dimethoxy- (6CI)
Furfuryl alcohol, tetrahydro-3,4-dihydroxy-2,5-dimethoxy-, acetate (6CI)
α-D-Glucopyranoside, 4-C-hydroxy-α-D-arabinofuranosyl (9CI)
Pentofuranoside, methyl 5-deoxy-3-C-(dimethoxymethyl)-4-C-methoxy- (9CI)
D-erythro-Pentofuranoside, methyl 5-deoxy-4-C-hydroxy-2,3-O-(1-methylethylidene)-, (4ξ)- (9CI)
D-erythro-Pentofuranoside, methyl 5-deoxy-4-C-methoxy-2,3-O-(1-methylethylidene)-, (4ξ)- (9CI)
α-D-Fructofuranose, 5-C-hydroxy-, 1,6-bis(dihydrogen phosphate) (9CI)
β-D-Fructofuranose, 5-C-hydroxy-, 1,6-bis(dihydrogen phosphate) (9CI)
α-D-ribo-Hexopyranosid-3-ulose, (4R)-4-C-hydroxy-β-D-arabinofuranosyl (9CI)
α-D-Galactopyranoside, (5S)-5-C-hydroxy-β-D-threo-2-pentulofuranosyl (9CI)
α-D-Glucopyranoside, (5S)-5-C-hydroxy-β-D-threo-2-pentulofuranosyl (9CI)
2,7,12,13-Tetraoxatricyclo[7.2.1.1^{3,6}]tridecane-4,5,9,10,11-pentol, 3-(hydroxymethyl)-1,6-dimethyl-, (4S,5R,10S,11R)-
β-D-Xylofuranose, 1,5-anhydro-4-C-(α-D-glucopyranosyloxy)- (9CI)
2-Furoic acid, tetrahydro-3,4-dihydroxy-2,5-dimethoxy-, -methyl ester, diacetate (7CI)
β-D-threo-2,5-Hexodiulo-2,6-pyranose, 5-hydrate, 5,2¹:5,3¹-dianhydride with 5-C-hydroxy-α-L-sorbofuranose (9CI)
D-erythro-Pentofuranose, 5-bromo-5-deoxy-4-C-hydroxy-2,3-O-(1-methylethylidene)-, (4ξ)-
D-erythro-Pentofuranose, 5-deoxy-4-C-hydroxy-5-iodo-2,3-O-(1-methylethylidene)-, (4ξ)-
3,4-Furandiol, 2-[(benzoyloxy)methyl]tetrahydro-2,5-dimethoxy-(9CI)
β-D-ribo-Pentofuranoside, methyl 5-bromo-5-deoxy-4-C-hydroxy-2,3,O-(1-methylethylidene)-
α-L-lyxo-Pentofuranoside, methyl 5-bromo-5-deoxy-4-C-hydroxy-2,3-O-(1-methylethylidene)-
α-D-ribo-Pentofuranoside, methyl 5-bromo-5-deoxy-4-C-hydroxy-2,3-O-(1-methylethylidene)-
β-L-lyxo-Pentofuranoside, methyl 5-bromo-5-deoxy-4-C-hydroxy-2,3-O-(1-methylethylidene)-
D-erythro-Pentofuranoside, methyl 5-bromo-5-deoxy-4-C-hydroxy-2,3-O-(1-methylethylidene)-, (4ξ)-
D-erythro-Pentofuranoside, methyl 5-deoxy-4-C-hydroxy-5-iodo-2,3-O-(1-methylethylidene)-, (4ξ)-
Pentofuranoside, methyl 5-deoxy-3-C-(dimethoxymethyl)-4-C-methoxy-2,3-bis-O-(trimethylsilyl)- (9CI)
Furo[3,4-d]-1,3,2-dioxaphosphol-4-ol, tetrahydro-2,6-dimethoxy-4,6-dimethyl-, 2-oxide (9CI)
D-erythro-L-ribo-5-Nonuto-5,2-furanose, 5,9-anhydro-1,6,7,8-tetradeoxy-3-O-[(1,1-dimethylethyl)dimethylsilyl]2-C-hydroxy-6,8-dimethyl-4-O-methyl-, (5S)- (9CI)
Pentofuranoside, methyl 5-deoxy-3-C-(dimethoxymethyl)-4-C-methoxy-, diacetate (9CI)
13,14-Dioxatricyclo[8.2.1.1^{4,7}]tetradecane-5,6,11,12-tetrol, 1,4,7,10-tetramethoxy-
3,4-Furandiol, tetrahydro-2,5-dimethoxy-2-methyl-, dibenzoate (7CI)
β-D-Ribofuranoside, (2-nitrophenyl)methyl 4-C-methoxy-
β-L-erythro-Hexofuranosid-5-ulose, methyl 6-deoxy-4-C-methoxy-2,3-O-(1-methylethylidene)-, (S)- (9CI)
α-D-Lyxofuranose, 4-C-ethoxy-5-O-(phenylmethyl)-, triacetate (9CI)
β-D-Lyxofuranose, 4-C-ethoxy-5-O-(phenylmethyl)-, triacetate (9CI)
β-D-Ribofuranoside, (2-nitrophenyl)methyl 4-C-methoxy-2-O-methyl-
α-D-Glucofuranose, 4-O-methyl-1,2:5,6-bis-O-(1-methylethylidene)-2,3,7-Trioxabicyclo[2.2.1]heptane-5,6-diol, 1,4-dimethyl-, dinitrate, (5-endo, 6-exo)- (9CI)
Galactitol, 2,5-anhydro-1,6-dideoxy-2,5-dimethoxy-3,4-di-C-methyl-, cyclic 3,4-(hydrogen phosphate) (8CI)
a-D-Tagatofuranoside, methyl 1,6-dideoxy-5-C-methoxy-3,4-di-C-methyl-, cyclic hydrogen phosphate (9CI)
β-D-Riboruranoside, methyl 4-C-(1-cyanoethoxy)-5-deoxy-2,3-O-(1-methylethylidene)- (9CI)
Phosphoric acid, methyl ester, cyclic 3,4-ester with tetrahydro-5-methoxy-2,3,4,5-tetramethyl-2,3,4-furantriol (7CI)
α-L-Tagatofuranose, 2,5-anhydro-1,6-dideoxy-5-C-methoxy-3,4-di-C-methyl-, cyclic 3,4-(methyl phosphate), (R)-(9CI)
α-L-Tagatofuranose, 1,6-dideoxy-5-C-methoxy-3,4-di-C-methyl-, cyclic 3,4-(methyl phosphate), (S)- (9CI)
Fructofuranose, O-α-D-galactopyranosyl-(1→6)-O-α-D-glucopyranosyl-6-t-(1→4)-, β-D- (8CI)
Raffinose-6′-t (8CI)
SaH-Oxireno[8,8a]naphtho[2,3-b]furan-5a,6-diol, decahydro-7,8a-dimethoxy-4,4a,6-trimethyl-, (1aR,4S,4aR,5aR,6S,7S,8aS,9aS)-
Molybdate(1-), [μ-[1,6-dideoxy-5-C-hydroxy-3,4-di-C-methyl-α-psicofuranosato(3-)-O²,O³:O³,O⁴]]-μ-oxotetraoxodi-, steroisomer (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-oxo-tetraoxodimolybdate(1-) (9CI)
1-Butanaminium, N,N,N-tributyl-, stereoisomer of [μ-[1,6-dideoxy-5-C-hydroxy-3,4-di-C-methyl-α-psicofuranosato(3-)-O²,O³:O³,O⁴]]-μ-oxo-tetraoxodimolybdate(1-), compd. with 1,1′-oxybis[ethane](2:1) (9CI)
9a H-4a,8-Epoxy-1,3-dioxolo[4,5]furo[2,3-d]oxepin-9a-ol, hexahydro-2,2,8-trimethyl-, [3aS-(3aα,4aα,8α,9aβ,9bα)]-(9CI)
α-Lyxofuranoside, methyl 3-C-[(benzoyloxy)methyl]-5-deoxy-4-C-methoxy-, 2-acetate (9CI)
2-Furoic acid, tetrahydro-3,4-dihydroxy-2,5-dimethoxy-, methyl ester, dibenzoate (7CI)
β-D-ribo-Heptofuranoside, methyl 4,6-anhydro-5,7-dideoxy-4-C-hydroxy-6-Cmethyl-2,3-O-(1-methylethylidene)- (9CI)
β-L-Sorbofuranose, 5-C-hydroxy-1,3:4,6-bis-O-(phenylmethylene)-, [1(R),4(R)]- (9CI)
β-L-Sorbofuranose, 5-C-hydroxy-1,3:4,6-bis-O-(phenylmethylene)-(9CI)
α-D-Glucofuranose, 4-O-methyl-1,2:5,6-bis-O-(1-methylethylidene)-, 3-acetate
α-D-Galactofuranose, 4-O-methyl-1,2:5,6-bis-O-(1-methylethylidene)-, 3-acetate
2,4,6-Metheno-2H-cyclopenta[g]furo[2,3,4-ij][2]benzopyran-2,5a,6a,9a,9b,9c(2aH,6H,7H)-hexol, tetrahydro-2a,6,9-trimethyl-4-(1-methylethyl)-, (2S,2aS,4S,5aR,6S,6aS,9R,9aS,9bR,9cS,10S)- (9CI)
Furo[2,3-d:4,5-d]bis[1,3]dioxole, tetrahydro-2,2,3a,6,6,7b-hexamethyl-, (3aα,4aα,7aβ,7bβ)- (9CI)
1,4a-(Epoxymethano)-4aH-xanthen-9 (2H)-one, 1,3,4,9a-tetrahydro-1,9a-dihydroxy-11-methoxy-
α-L-Sorbofuranose, 5-C-methoxy-1,3:4,6-bis-O-(phenylmethylene)-, [1(R),4(R),5S]- (9CI)
Furo[2,3-d:4,5-d]bis[1,3]dioxole, tetrahydro-2,2,3a,4a,6,6-hexamethyl-, (3aα,4aβ,7aβ,7bα)- (9CI)
Spiro[furan-2(3H),4′(3′aH)-furo[3,4-d][1,3]dioxole], 6′,6′a-dihydro-6′-methoxy-2′,2′-dimethyl-, (2R,3′aS,6′R,6′aR)-
Octofuranosiduronic acid, methyl 3,6-anhydro-5-deoxy-4-C-methoxy-6-C-(methoxycarbonyl)-, methyl ester (9CI)
β-L-erythro-Hexofuranosid-5-ulose, methyl 6-deoxy-4-C-methoxy-2,3-O-(1-methylethylidene)-, oxime, (4R)- (9CI)
2-Propanone, 1-hydroxy-1-[tetrahydro-6-hydroxy-2,3a,5-trimethyl-5,2-(epoxymethano)furo[2,3-d]-1,3-dioxol-8-yl]-, [2R-[2α,3aβ,5α,6β,6aβ,8S*(R*)]]- (9CI)
β-L-erythro-β-L-lyxo-Decofuranos-7-ulo-7,10-furanose, 10-C-(acetyloxy)-3,7-anhydro-5,6-dideoxy-, tetraacetate, (10R)- (9CI)
Inulobiose, octaacetate (5CI)
2,3,7-Trioxabicyclo[2.2.1]heptane-1-acetonitrile, 5-[[(1,1-dimethylethyl)dimethylsilyl]oxy]-6-methoxy-4-methyl-, (1R,4S,5R,6R)-
D-gluco-Nonitol, 2,5-anhydro-1,6,7,8-tetradeoxy-3-O-[(1,1-dimethylethyl)dimethylsilyl]-2,5-C-epidioxy-6,8-dimethyl-4-O-methyl-, (2ξ,5ξ)- (9CI)
β-D-Lyxofuranose, 4-C-ethoxy-2,3-O-(1-methylethylidene)-5-O-(phenylmethyl)-, acetate (9CI)
2-Heptenal, 6-[(5S,6R)-5-hydroxy-6-methoxy-4-methyl-2,3,7-trioxabicyclo[2.2.1]hept-1-yl]-4-methyl-, (2E,4S,6R)-
Spiro[1,2-dioxin-3(6H),5′(3′aH)-furo[2,3-d][1,3]dioxole]-6,6′-diol, 6′,6′a-dihydro-2′,2′,6-trimethyl-, 6′-acetate, (3R,3′aS,6′R,6′aR)-
Spiro[1,2-dioxin-3(6H), 5′(3′aH)-furo[2,3-d][1,3]dioxole]-6,6′-diol, 6′,6′a-dihydro-2′,2′,6-trimethyl-, 6′-acetate, (3S,3′aS,6′R,6′aR)-
5,8-Epoxy-1,4-dioxino[2,3-d][1,2]dioxin, hexahydro-4a,5,8,8a-tetramethyl-, (4aR,5S,8R,8aS)-rel-
5,8-Epoxy-1,4-dioxino[2,3-d][1,2]dioxin, hexahydro-4a,5,8,8a-tetramethyl-, (4aR,5R,8S,8aS)-rel-
5,8-Epoxy-1,4-dioxino[2,3-d][1,2]dioxin, hexahydro-4a,5,8,8a-tetramethyl-
4H-1-Benzopyran-4-one, 2-(3,4-dihydroxyphenyl)-5,6-dihydroxy-7-[(4-C-hydroxy-α-D-ribofuranosyl)oxy]- (9CI)
Furo[2,3-d:4,5-d]bis[1,3]dioxole, tetrahydro-2,2,3a,4a,6,6,7a,7b-octamethyl-, (3aα,4aα,17aβ,7bβ)- (9CI)
6,9-Epoxy-2H-o-dioxino[4,5-b][1,4]dioxepin, hexahydro-5a,6,9,9a-tetramethyl-, stereoisomer (8CI)
6,9-Epoxy-2H-o-dioxino[4,5-b][1,4]dioxepin, hexahydro-5a,6,9,9a-tetramethyl-, stereoisomer (8CI)
L-gulo-Nonose, 5,8-anhydro-2,3,4,9-tetradeoxy-7-O-[(1,1-dimethylethyl)dimethylsilyl]-5,8-C-epidioxy-2,4-dimethyl-6-O-methyl-, (5ξ,8ξ)- (9CI)
α-L-Sorbofuranose, 5-C-methoxy-1,3:4,6-bis-O-(phenylmethylene)-, acetate, [1(R),4(R),5S]- (9CI)
Spiro[1,2-dioxin-3(6H),5′(3′aH)-furo[2,3-d)[1,3]dioxol]-6-ol, 6′,6′a-dihydro-6′-methoxy-6-methyl-2′-(trichloromethyl)-, (2′S,3S,3′aS,6′R,6′aR)-
Spiro[1,2-dioxin-3(6H),5′(3′aH)-furo[2,3-d)[1,3]dioxol]-6-ol, 6′,6′a-dihydro-6′-methoxy-6-methyl-2′-(trichloromethyl)-, (2′R,3R,3′aS,6′R,6′aR)-
7,10-Epoxy[1,2]dioxino[4,5-b][1,4]dioxocin, octahydro-6a,7,10,10a-tetramethyl-
Furo[2,3-d:4,5-d]bis[1,3]dioxole-3a(4aH)-carboxylic acid, dihydro-2,2,6,6-tetramethyl- (9CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, (3aR,3′aS,3bR,5S,6′S,6′aR,7aS,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, (3aR,3′aS,3bR,5S,6′S,6′aR,7aR,8aR)- (9CI)
Spiro[1,2-dioxin-3(6H),5′(3′aH)-furo[2,3-d][1,3]dioxole]-6,6′-diol, 6′,6′a-dihydro-2′,2′,6-trimethyl-, 6,6′-diacetate, (3S,3′aS,6′R,6′aR)-
Spiro[1,2-dioxin-3(6H),5′(3′aH)-furo[2,3-d][1,3]dioxole]-6,6′-diol, 6′,6′a-dihydro-2′,2′,6-trimethyl-, 6,6′-diacetate, (3R,3′aS,6′R,6′aR)-
2H-Naphtho[1,2-b]pyran-2-one, 6-[[5-C-(α-D-glucopyranosyloxy)-β-D-fructofuranosyl]oxy]-7-hydroxy-3-methyl-
Spiro[furo[3,4-d]-1,3-dioxole-4(3aH),5′(4′H)-isoxazole], 6,6a-dihydro-6-methoxy-2,2,3′-trimethyl-, (3aS,4R,6R,6aR)-
Spiro[1,2-dioxin-3 (6H),5′(3′aH)-furo[2,3-d][1,3]dioxole]-6,6′-diol, 6′,6′a-dihydro-6-methyl-2′-(trichloromethyl)-, 6′-acetate, (2′S,3S,3′aS,6′R,6′aR)-
Spiro[1,2-dioxin-3(6H), 5′(3′aH)-furo[2,3-d][1,3]dioxole]-6,6′-diol, 6′,6′a-dihydro-6-methyl-2′-(trichloromethyl)-, 6′-acetate, (2′R,3R,3′aS,6′R,6′aR)-
ε-D-Glucofuranose, 4-C-hydroxy-1,2:5,6-bis-O-(1-methylethylidene)-, 3-(3-chlorobenzoate)
Spiro[5H-1,3-dioxolo[4,5]furo[3,2-b]pyran-5,5′(3′aH)-furo[2,3-d][1,3]dioxol]-6′-ol, 3a,6,6′,6′a,7,8a-hexahydro-2,2,2′,2′-tetramethyl-, (3aR,3′aS,5S,6′S,6′aR,8aR)-
Spiro[furo[3,4-d]-1,3-dioxole-4(3aH),5′(4′H)-isoxazole], 3′-ethyl-6,6a-dihydro-6-methoxy-2,2-dimethyl-, (3aS,4R,6R,6aR)-
1,4-Epoxy-o-dioxino[4,5-b][1,4]benzodioxin, 1,4,4a,10a-tetrahydro-1,4,4a,10a-tetramethyl-, stereoisomer (8CI)
1,4-Epoxy-o-dioxino[4,5-b][1,4]benzodioxin, 1,4,4a,10a-tetrahydro-1,4,4a,10a-tetramethyl-, stereoisomer (8CI)
1,4:6,9-Diepoxy-p-dioxino[2,3-d:5,6-d]bis-o-dioxin, octahydro-1,4,4a,5a,6,9,9a,10a-octamethyl-, stereoisomer (8CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2′,2′-tetramethyl-, (3aR,3′aS,3bR,5S,6′S,6′aR,7aS,8aR)- (9CI)
Spiro[5H-1,3-dioxolo[4,5]furo[3,2-b]pyran-5,5′(3′aH)-furo[2,3-d][1,3]dioxol]-6′-ol, 6′-(aminomethyl)octahydro-2,2,2′,2′-tetramethyl-, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, octahydro-2,2,2′,2′-tetramethyl-, 6′-acetate, (3aR,3′aS,3bS,5S,6′S,6′aR,7aR,8aR)-
Spiro[5H-1,3-dioxolo[4,5]furo[3,2-b]pyran-5,5′(3′aH)-furo[2,3-d][1,3]dioxol]-6′-ol, 6′-(1-aminoethyl)-3a,3b,6,6′,6′a,7,7a,8a-octahydro-2,2,2′,2′-tetramethyl-, (3aR,3′aS,3bS,5S,6′S,6′aR,7aR,8aR)-
L-glycero-β-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-gulo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-β-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
L-glycero-α-D-allo-Octofuranuronic acid, 4-C-(acetyloxy)-3,6-anhydro-5-deoxy-6-C-(methoxycarbonyl)-, methyl ester, 1-acetate 2,7-dibenzoate (9CI)
β-L-threo-Pentofuranose, 4-C-(acetyloxy)-5-deoxy-5-fluoro-1,2-O-(1-methylethylidene)-, 4-methylbenzenesulfonate, (4ξ)- (9CI)
α-D-Glucopyranoside, methyl 6-deoxy-4-O-(4-C-hydroxy-2,3,5-tri-O-methyl-α-D-arabinofuranosyl)-2,3-di-O-methyl-6-[[(4-nitrophenyl) sulfonyl]amino]-
Spiro[furo[3,4-d]-1,3-dioxole-4(3aH),5′(4′H)-isoxazole], 6,6a-dihydro-6-methoxy-2,2-dimethyl-3′-[(3aR,4R,6R,6aR)-tetrahydro-6-methoxy-2,2-dimethylfuro[3,4-d]-1,3-dioxol-4-yl]-, (3aS,4R,6R,6aR)
Spiro[furo[3,4-d]-1,3-dioxole-4(3aH),2′(3′H)-pyrrolo[1,2-b]isoxazole], hexahydro-6-methoxy-4′,5′-bis(methoxymethoxy)-2,2-dimethyl-, (2′R,3aS,3′aS,4′S,5′S,6R,6aR)-
5,2,9-Ethanylylidene-1-benzoxepin-8,11(2H)-dione, 4-[(2R,3R)-3,4-dihydro-3,5,7-trihydroxy-2H-1-benzopyran-2-yl]-6-[(2S,3S)-3,4-dihydro-3,5,7-trihydroxy-2H-1-benzopyran-2-yl]-5,5a,9,9a-tetrahydro-2,9,9a,10-tetrahydroxy-, (2R,5S,5aS,9S,9aS,10R)- (9CI)
Spiro[5H-1,3-dioxolo[4,5]furo[3,2-b]pyran-5,5′(3′a H)-furo[2,3-d][1,3]dioxol]-6′-ol, 3a,6,6′,6′a,7,8a-hexahydro-2,2,2′,2′-tetramethyl-, 6′-acetate, (3aR,3′aS,5S,6′S,6′aR,8aR)-
Spiro[furo[3,4-d)-1,3-dioxole-4(3aH),5′(4′H)-isoxazole], 6,6a-dihydro-6-methoxy-2,2-dimethyl-3′-phenyl-, (3aS,4R,6R,6aR)-
1,4-Epoxycyclobuta[5,6]-p-dioxino[2,3-d)-o-dioxin, 1,4,4a,5a,7a,8a-hexahydro-1,4,4a,5a,6,7,7a,8a-octamethyl-, stereoisomer (8CI)
1,4-Epoxycyclobuta[5,6]-p-dioxino[2,3-d)-o-dioxin, 1,4,4a,5a,7a,8a-hexahydro-1,4,4a,5a,6,7,7a,8a-octamethyl-, stereoisomer (8CI)
Spiro[furo[3,4-d)-1,3-dioxole4(3aH),6′-[6H]1,2]oxazine]-3′-carboxylic acid, hexahydro-6-methoxy-2,2-dimethyl-, ethyl ester, (3′ R,3a5,4R,6R,6aR)-
Spiro[furo[3,4-d)-1,3-dioxole-4(3aH),5′-isoxazolidine], dihydro-6-methoxy-2,2-dimethyl-2′-(phenylmethyl)-, (3aS,4R,6R,6aR)-
2H-Naphtho[1,2-b]pyran-2-one, 7-(acetyloxy)-3-methyl-6-[[1,3,4,6-tetra-O-acetyl-5-C-[(2,3,4,6-tetra-O-acetyl-α-D-glucopyranosyl)oxy]-β-D-fructofuranosyl]oxy]-
Spiro[furo[3,4-d)-1,3-dioxole-4(3aH),5′(4′H)-isoxazole), 6,6a-dihydro-6-methoxy-2,2-dimethyl-3′-(4-methylphenyl)-, (3aS,4R,6R,6aR)-
3,6-Epoxy-2H,8H-pyrimido[6,1-b][1,3]-oxazocine-8,10(9H)-(dione, 3,4,5,6-tetrahydro-4-hydroxy-, acetate (ester)(7CI)
Spiro[5H-1,3-dioxolo[4,5]furo[3,2-b]pyran-5,5′(3′aH)-furo[2,3-d)[1,3]dioxol]-6′-ol, octahydro-2,2,2′,2′-tetramethyl-, 6′-benzoate, (3aR,3′aS,3bS,5S,6′S,6′aR,7aR,8aR)-
Spiro[5H-1,3-dioxolo[4,5][furo[3,2-b]pyran-5,5′(3′aH)-furo[2,3-d) [1,3]dioxol]-6′-ol, octahydro-2,2,2′,2′-tetramethyl-6′-(nitromethyl)-, (3aR,3′aS,3bS,5S,6′S,6′aR,7aR,8aR)-
Spiro[furo[3,4-d)-1,3-dioxole-4(3aH),6-[16H]1,2]oxazine]-3′-carboxylic acid, 4′,5′,6,6a-tetrahydro-6-methoxy-2,2-dimethyl-, ethyl ester, (3aS,4R,6R,6aR)-
Hexanoic acid, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Acetamide, N-[1-[(2′R,3′R,3aS,3′aS,5′S,6S,6aR,7′aR)-2′,3′-bis(acetyloxy)-2′,3′,3′a,6,6′,6a,7′,7′a-octahydro-6-hydroxy-2,2-dimethylspiro[furo[2,3-d]-1,3-dioxole-5 (3aH),5′-[5H]furo[3,2-b]pyran]-6-yl]ethyl]-
Spiro[5H-1,3-dioxolo[4,5]furo[3,2-b]pyran-5,5′(3′aH)-furo[2,3-d)[1,3]dioxol]-6′-ol, 3a,6,6′,6′a,7,8a-hexahydro-2,2,2′,2′-tetramethyl-6′-(nitromethyl)-, (3aR,3′aS,5S,6′S,6′aR,8aR)-
Spiro[5H-1,3-dioxolo[4,5]furo[3,2-b]pyran-5,5′(3′aH)-furo[2,3-d)[1,3]dioxol]-6′-ol, 3a,6,6′,6′a,7,8a-hexahydro-2,2,2′,2′-tetramethyl-, 6′-benzoate, (3aR,3′aS,5S,6′S,6′aR,8aR)-
Spiro[furo[3,4-d)-1,3-dioxole-4(3aH),5′(4′H)-isoxazole], 3′-(2,6-dichlorophenyl)-6,6a-dihydro-6-methoxy-2,2-dimethyl-, (3aS,4R,6R,6aR)-
1,6,11,14,16,20,23,24-Octaoxahexaspiro[3.0.3.0.0.4.0.3.0.3.1.1]tetracosane
Hexanoic acid, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3)dioxol]-6′-yl ester
Benzoic acid, 4-chloro-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Benzoic acid, 4-bromo-, (3aR,3′aS,3bS,5S,6′S,6′aR,7aR,8aR)-octahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Spiro[5H-1,3-dioxolo[4,5]furo[3,2-b]pyran-5,5′(3′aH)-furo[2,3-d][1,3]dioxol]-6′-ol, 3a,6,6′,6′a,7,8a-hexahydro-2,2,2′,2′-tetramethyl-6′-(1-nitroethyl)-, (3′aS,3aR,5S,6′S,6′aR,8aR)-
Sorbofuranose, 2,3:4,6-di-O-isopropylidene-, p-toluenesulfonate, α-L-(7CI)
Benzoic acid, 4-chloro-, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Benzoic acid, 4-bromo-, (3aR,3′aS,5S,6′S,6′aR,8aR)-3a,6,6′,6′a,7,8a-hexahydro-2,2,2′,2′-tetramethylspiro[5H-1,3-dioxolo[4,5]furo[3,2-b]pyran-5,5′(3′aH)-furo[2,3-d][1,3]dioxol]-6′-yl ester
Spiro[furo[2,3-d]-1,3-dioxole-5 (3aH),6′-[6H]1,2]oxazine]-3′-carboxylic acid, hexahydro-2,2-dimethyl-6-(phenylmethoxy)-, ethyl ester, (3′R,3aS,5R,6R,6aR)-
Spiro[furo[3,4-d]-1,3-dioxole-4(3aH),6′-[6H-1,2]oxazine]-2′,3′(3′H)-dicarboxylic acid, tetrahydro-6-methoxy-2,2-dimethyl-, 2′-(1,1-dimethylethyl) 3′-ethyl ester, (3′R,3aS,4R,6R,6aR)-
Spiro[furo[2,3-d]-1,3-dioxole-5(3aH),6′-[6H]1,2]oxazine]-3′-carboxylic acid, 4′,5′,6,6a-tetrahydro-2,2-dimethyl-6-(phenylmethoxy)-, ethyl ester, (3aS,5R,6R,6aR)-
β-D-Galactofuranose, 3,4-O-[(acetylamino)phenylmethylene]-4-C-hydroxy-, 1-acetate 2,5,6-tribenzoate (9CI)
β-D-Galactofuranose, 3,4-O-[(2,5-dioxo-1-pyrrolidinyl)phenylmethylene]-4-C-hydroxy-, 1-acetate 2,5,6-tribenzoate (9CI)
Osmium, tetraoxotetrakis(pyridine)[μ-[1-(tetrahydro-2,3,4,5-tetrahydroxy-2-furanyl) ethanonato(4-)]]di-, stereoisomer (9CI)
β-D-Galactofuranose, 3,4-O-[(1,3-dihydro-1,3-dioxo-2H-isoindol-2-yl)phenylmethylene]-4-C-hydroxy-, 1-acetate 2,5,6-tribenzoate (9CI)
Piperazinone, 4-[(dihydro-2,2,6,6-tetramethylfuro[2,3-d;4,5-d]bis[1,3]dioxol-3a(4aH)-yl) carbonyl]-6-ethyl-1-[(4-methylphenyl)methyl]- (9CI)
Quinoline, 5,7-dichloro-1-[(dihydro-2,2,6,6-tetramethylfuro[2,3-d:4,5-d]bis[1,3]dioxol-3a(4aH)-yl) carbonyl]-4-(4-fluorophenoxy)-1,2-dihydro-(9CI); or a combination thereof.

124. The composition of claim 123, comprising polyolprepolymer-2 and wherein the carrier is ethyl alcohol.

125. A method of reducing a population of pathogenic microorganisms on a surface, the method comprising applying to the surface a composition comprising: (a) one or more germinants; (b) polyolprepolymer-2 or poly(styrene-co-maleic anhydride) copolymers (SMA); (c) one or more nanoscale particle having a particle size of about 5 nm to about 200 nm; and (d) a carrier.

126. The method of claim 125, further comprising exposing the surface to visible or ultra-violet light.

127. The method of claim 125, further comprising heating the composition prior to or after applying the composition to the surface.
